SEQUENCE LISTING (13 sequences)

SEQ ID NO: 1 - DNA, 21 bp, Artificial Sequence
  Synthetic Polynucleotide
cccacaggggccctggctatg

SEQ ID NO: 2 - DNA, 21 bp, Artificial Sequence
  Synthetic Polynucleotide
ctaaacaccccatggatctag

SEQ ID NO: 3 - DNA, 21 bp, Artificial Sequence
  Synthetic Polynucleotide
catttgctaagtgtatgatgt

SEQ ID NO: 4 - DNA, 21 bp, Artificial Sequence
  Synthetic Polynucleotide
ccctaccacagggtcagcctc

SEQ ID NO: 5 - DNA, 22 bp, Artificial Sequence
  Synthetic Polynucleotide
ccattatatccagggactctcc

SEQ ID NO: 6 - DNA, 21 bp, Artificial Sequence
  Synthetic Polynucleotide
taccacagggtcagcctcccc

SEQ ID NO: 7 - DNA, 24483 bp, Homo sapiens
tgccatgggctaatctctcaggtgagtgttgtggacgtctcaccctccaatatcgtgctggatgacttctttctgtggcccccaggatagctgcactcagcatggccaaagttgaacctcccatgtccatccccaaacccacttctctttagccccatctctaaagtcagaaccagcttgtgggtctccaccttgacctccccaccttctccagatctcataaatcacccactcttgtccctctttctcccaaccagggctcatttggtgcccttttctccgtcctgaccttggtcatgcctgggccctcacctcctccctggtcactgaaccatcctcaccttttgcctccctgtctccccagcacaggcctccagactctgcttctagctgcctcctcgtccagtgcctccacagtctgaacagccttgtctccttgtttcctctccattgctggagaacaccaccgtctggactgaaggccttgcacgttctatcctccactggactctcctgatccctcctttctctgtgtcacccaaagtctccccacacccactgggccaagccctctatgactctgagactttgcatgtgtagttccgtttcctgaaatgcccttccctcccattcctgccaagtccaggtcctgattgtccttcaggctcagtcttaaatgtcacccgagtgtgtgtgtgtgtgtgtgtgtgtgtgtgtgtgtgtgtgtgtgtttcttgtatgatattgagggaaggagtgacatttattcattaatctatagattctttggtaactatactgaacacctactatgtgccagcattgttgtaggtgccaggggttcactggtagacatagcatcagaaatctcagtattaagaagatgttctgggctgggcatcgtggctcacacctgtaatcccagcactttgggaggctgaggtagtggatcacctgagatcaggagttcgagaccggcctgaccaacaaggtgaaaccccatctctactaaaaatcaaaaattaggtgggcatggtggcatgtgcctgtagtcccagctactcgggaagctgagacaggagaattgcttgaacctgggaggcagaggttgcagtgcgagactctgtctcagaaaaaaaaaaaaagatattctgaagacaagatggacataaaaaaaggaataagcaatagaaagtatttaactatataggatggcaatatatgtttagagaaaaatcatgcagggaaagaagcataagatatactggggaaccatctctgtacctgatgatgggaagagattatatttgggctaacacatgaaggtggtgaggaagcaggctctgtgtgtatataagcaaaagcatcccaggaagaaggaaaagggggaagagcaaatgcattgctcagaggcagcagtaggcctgggtgttgcaaacacacccagtaacccatgtctaagagcagagggaggcagaggagtcaagcggtggaaaggacactgcagaccacctggtgccctgcaggccacacaggaacctagagcctttgtggagaggagagactcaactggactggatcccttggctccctggcgagagcaggcctgagcgggtcaaggcacctacagacagactgggagagcgctgctggatcctccaggaagaggaatgaggtcttgaaccagacaggcagggttggagtgaggatgtgtttttggatgtatttggaaggtcaagctcacaggattttctaatggatatagtcattgaggggaagggaagagcatggataaagccagaatgttcctctgaacatctcactctttcgaaactccaatgtcttttttgtttttgttttgttgttgttgttgttttgttttttttttttcttgagacggagtcactctgttgcccgggcttgagtgcgatggtgtgatcttggctcactgcaacctctgcttcccaggttcaagctattctcctgcctcagcctcccgagcagctaggattacaggcgcttgccaccacacccagctaatttttgttgttgttgtatttttagtggagacagggtttcaccatgttggtcaggctggtcttgaactcttgacctcaagtcatccaccaatcttggcctcccaaagtgctgagattacaggcatgagctaccactcctggcctccaatgtctattattccacactctatgcccatgtatacacattatttaactacacttatacatgagaacatatgatatttgactttccacttctgaattatttcagttaagataaccttcagttccaaccatgttgcttgaaaagacatgatttcattctttttcatggctgggtagtattccattgtataacccccattttctttatccaatcatccatggataagcattaggttgattccatatctttgctgttgtgaatagtgctgctacaaacatacgagtgcaggtatgtttttgatataataatttcttttcctttacccagtagtgggatcattggatcaaatggtagttctatttttggttatttgagaaatctctctgttttccgtagaggctgtaataatttaccttcccatgaacactgtataagcatttcctgttctccacatcctcaccaacatctgttatttcttgactttttaaaaatagccattctatctggtggaagatgatttctcactgcggttttaatttgcatttatctgatgattagtgatgttgaacctgtttcacatgcttcttggccacttgtatgtcttcttttgaaaactgcctattcatgtcttttgcccactatttaatgggattttttgttttttattattaagctgcttcagtttcttgtacattctggatattagtctcttatcagattcacagtttgcaaatattttcttccattctgtaggttgtctgttcactctgttgattattttttatgctatgcagaagctttttagtttaattacatcctgtttatctattttttgttttgttggatttgctttgagatcttagttgtgaattctttgcctacgccaatgtccagaaaagattttcctaggttttcttcagtatttctatagtttcaggtcttacatctaagtttttaacccatcttgagttaatgtttgtgtatggtgagagataggggtccattgcattattctgcacatggctatccaattctcccagcaccgtttaattaataaggtgtcctgtccccagtgtatgtttttgtcgactttgtcaaaagtcaggtagctgtaagtatatggctttatttctgggttctctattctgtttcattaatctgtgtgtctatttttataccagtaccatgcttttttgtttactatagccttgtggcataatttgaagtcaggtaatgtgatgtctctagctttgttctttttgcttaggactgctttggtaatttgggttctttttgtttctatatgaattttagaattttttttcaaattatgtgaaatatgacattggtattttgataggaattgcattgaatctgtagattgctttgggcagtacggtcattttcaaggtattgattcttctgatccatgagcacgggatgctccatttttccattcatctgtgtcatctacaacttatttcatcagtattttgtggtttgccttgtactgatctttcacctccttggttaaatatattcctaggtatttttctgttgcaattgaaaatgagattgacttcttgatttttcttctgttagattactgttggtgtatagaagtgctactgatttttgtacattgattttgtatcctgaaattttactgaattaatttattaaatctaggagtcttttgaaggagtccttagagttttctagatttaagatcctatcatcagcaaacagagataatttgacttcctcttttccaatgtgggcgccttttgtttctttctcttgcctgattaattgctctagataggactttcagtatcaggttgaatagcaatgaagagagtgatcatccttgtcttgttccagttcttagggggaatgctttcagtttttaccctttcagtatgatgttggctgtgggtttgtcatagatggctcttattattttgagatatgttcctttaatgtctagtttgttgagggtttttaccatgaagagatgttggattttatcaaaagctttttctttgtctagtgggatgattatatggtttttgcttttaattctgtcaatgtgatgaatcacatttattgatttgcatgtacggaaacatccttgcatccctggaataaagcctacttgatcgtgacgaattatctttttgatgtgctgttgaatttggtttgctagtattttgttgaggatttttgcgtctatgttcatcagggatattggactgtaatttacttttctcattgtgtccttgctaggttttgatatcacggtcatactggcttcatagaatgagtcagagaggattccctcctcctgaatattttgggagagttattctttgtacatttggtagaatttgactgtgaaaccatctggtcctggacttctatttgttgagagatttttttctattgctggttcaatctcactacttgttattggtctgttcagcatttctatttcttcctgattcaatcttggcagaatcttatggttctaggaatttatgcatttcctccaggaaaagttcctctccatgttttgaaacaaaagatattattctctcacagttctggaggctagaaatttgaaatcagtaaggccaggcagaaatgaaggtgttggttggactacaacactccagagacttgagaggatccatacctcgcttcttccacattattgtggctggtgtcattccttgaattttggctggactgctcctacttctgtctctggggccacatggcctcgcctccttctgtgtatatcacatctccctctgcttttctcttatcaggactcttgtaggactcttgtgattagatgtaagggttgactgcataatccaggatttccactggtcaagatcattaatttaattaaagttgtaagaatacctataaagtaacaatacaggctcaagaaactaagacccagttttctttgggggtcattattcagattactgagccgctgtgtgatcttctgccccctaggaggcccccatctgccctccagaccttgccttctctttctattactttctctgttctttccgtcctgacacactagtgggccaactcatgtaacgcgtatttatgggaatggggtctattataccataaaagacactgataagacagccaaaagataagttacagggtgataacacctgaatataggtatctgatgaatgacttgtatccagaatatatcaaggactctcaaaacaaaaacataagtaaaccacaacagcaacaacaacaacaaaaggcccaattttgtgtaaatgaaaaagcagccactcaacaataacaaaaaataaacaacacttaaaaatggacagagggtgtgaatcacatttctcaaaaaaatatagtactcaaggccctcaccagaagcaaatgccagcaccacatttcttgcacagcctgcagaactgtgagccaaaatatatctgttttcttcataaattactcagtctcagatattcctctatagcaagccaagaatgaactaacacaatattttaaaaaggtagccaaaggttttatcagccacctcaccagaaaagaaaaacagatggcaaacaggctcatgaaaacatactaaagatcatttgtccttaggtaattataaatcaggagaataagcattcttaggtagcattacatacctgagaacataactagaaaacaaaacaaaaataaaaagaaatgtttaaatattctgataatatcaaatgttaacaaggatgtgaaacaatcagaattcacacataactcctggtgggaatacaaaatggcacatcaactttggagaacagtttgacaatttcttataaatttaaactgaacacttaccataggaccaggcacctgactcctaagtatttacccaagagcaataaaaacttacagttacacacagaaacctgcatgccaatatttataacaacattatttcatagtcacccaaacctgaaaacaatctgaatgctcctcaaatggtaaatgcatacaaaatggggggtccataaaaacagtgagttacgagtcagcaattaaagggaacaaactatagattcatacactcaaccacatgattgaacctcagaggcactgcaccaagtaaatgaagccagactttggaggcaacattttgtatgattccatttatacgacattctgtgaaaaggcaatacagtagggatggaaaacatatcagcattttttcatggactaagtgtggagggagggttgactacccagagacaggaggaaggaacgtttggttaatgacactcttcagtgtcttcacagtggttgtgggtatataattatatacaactctcaaaccatatcaaaattgcatatgataaagagttaattcaactgtatctaactttaaataaattaaaaaatttaaaacactggatgccactatgctctcactagaattgttcaaatataaagcaggacagtatcatacgtcgacaagggtgtggagctactggaattctggcacatggctaggaggagtggtaaaaagtcaaaaccattttggacaacagtttggcaattcctactaaggttaaacttacaagtagtcattagtctgactatttatcttcctgtgtataccaattagtataaaaccatgtatgtaaaaagacttgtacctgaatacctgtagggagaaaactggaaaatccttgtctatcaataggtgagaaataaaacattatagtatacacataaatggaatactacttaacaatatcacattttgaaatacagacacaaacaacgtggttcagtttcaaaaaatattatgctgggtcataggatacagacacaaattaacacatctctaggagattttcaaatagactaactcacaatgaaagaggacatatcagtggttgcccagcatagaaggtgggaagaaaagcccttggcatactagaaatgctgtatatctgcagtgtggtgagtatacaggctgtacatactcgtcaaatctcatgcaaatgtgaacttcaagtaggtgtgctttatttcatgtaaattatacctaattaaattcattaaagaaacacagtaagactcacatattcatatttgatcttctggtccatgaacatagtatgactttattgttaaaactaaatgtggctgggcacagtgcctcatgcccatagtcaaagcactttggaggtcgaggtgggaggattgcttgaacccaggagttcaagatcagcctgggggaacatagcaagaccccatctctcctaaacataaaaacaaaaagttactccggcctggtagtatgagcctgtgatcccagctactagagaggatgacgtgggagagtcacttgagtctgggaggtcaaggctgcagtgaactgtggtcaggccggtgcactccagcctgggcaacaaagcaagaccctgtcttaaaaaaaaaagcactaaatgtattgaattttctattacgtgccacatgctaaatatttaaataacttacaacagatattatttactggagacttaatttgttgtatatcaaatgaggatgcagctacctctctattagtaaggatgaatgaagagttatctagatactattgtgtgtagagatttgacaaaggtatggtggcaagtactaatatgtcccagggatcaacggtttgctggatgcatactacacagagtgatatggaaaaccatgtagctggacctaaagtttaattttctctgtacactccactcatgttgtctggaaatattatgtctttgggccatatttaactgtgagaagcagatttttacatgaaagaaaatgttgatccatatgatgagcctctaaaaaaacttgcttctgcccacactgaaccaaatggcagcatgaatggaaaagaagttttttctgcactggttcatagaacatgtgttctcccttttgtttcacaggaagatgctgcatggttcttgttttggagatgtcattgacactgtgcaaatcagtgtactaggtgccaggggaaaattctatagtcatatttcttttaaaatttccaatatgctaccatgatacatggcaaattcccaacttttcacagctctaaagccagacagcatgtgtgtgtgtggtgaggacatgaggctatggatggggagtatcttaagggcaggaatcaggtcagtggcatctcttctcaagccccacctatacacaaaggctcactgtaagaattaaagaaagaggaaagaaacacgaatggtggctcaccagtcaagacagattttagagaaaacaaacctgagaggagtttctggccgagttaagtcagaggcacactctcttacagactaagagtttttaaggatttagggtgggagagtttatcagagacttggactgcttctgagtctctttgttgtgcttatctgggagagagaggtgtgtgtctgtacccatacatctttctgcagctacaggcatacccactgagtctgcttttagcttccctatcttagtgcacctgaagggaaaggaatttgcttattaaggcccactgttttactagggccattgtatgagggtgaagtttggcagtaactcaagagactctccccctacctccctctgtgcccgagctgtcttatctgtgtttcactgtctgctctttctggttgcttgtagttagcagagaagtgatttccttgaaatgcatgaggctagaaaggaaattggaacttaaagaggcagtgtttctccgagatgacggtgctcctgctctgtcactcactatatctttagtgaatgaatgaataaatgaatggctgccaccttcactttacctaggactcttctttgtttctctgccccagacactcacacggaccctatagaatcctatatcctgctggactaaacaccccatggatctaggtgaggctgcgactcgggccaggccagcagtgatcagccctggtgtaaacagttgtgcacagaaaagcacctcagagtggaaggactttagacatggggttcgcatgtctcagatggccctgaaggtactgatccaggctctggtgctcctggaaggcaagactcagattctgctccatctcctcccatctctgggcgggtctctggcatctctggcccatgagggtcaatctgtgtggaggggacaagctctgagcatgtgtgggtctgaggttcctcttccatgcagggctgaggtctcctgctcctccccagcttcctgtccggccctgtagtccttcccctccactcccttcctcttttctgctcacacaggaagccctggaagctgcttcctcagacatgccgctgctgctactgctgcccctgctgtgggcaggtgagtggctgtggggagaggggttgtcgggctgggccgagctgaccctcgtttccccacaggggccctggctatggatccaaatttctggctgcaagtgcaggagtcagtgacggtacaggagggtttgtgcgtcctcgtgccctgcactttcttccatcccataccctactacgacaagaactccccagttcatggttactggttccgggaaggagccattatatccagggactctccagtggccacaaacaagctagatcaagaagtacaggaggagactcagggcagattccgcctccttggggatcccagtaggaacaactgctccctgagcatcgtagacgccaggaggagggataatggttcatacttctttcggatggagagaggaagtaccaaatacagttacaaatctccccagctctctgtgcatgtgacaggtgaggcacaggcttcagaagtggccgcaagggaagttcatgggtactgcagggcagggctgggatgggaccctggtactgggaggggtttaggggtaaagcctgtcgtgcttagcgggggagcttgaccagaggttgatcttctctcaggccctcacctggaccctccctcctgattctgcatcccctctttctcctcactagacttgacccacaggcccaaaatcctcatccctggcactctagaacccggccactccaaaaacctgacctgctctgtgtcctgggcctgtgagcagggaacacccccgatcttctcctggttgtcagctgcccccacctccctgggccccaggactactcactcctcggtgctcataatcaccccacggccccaggaccacggcaccaacctgacctgtcaggtgaagttcgctggagctggtgtgactacggagagaaccatccagctcaacgtcacctgtaagtgctgggccaggatgctggggtccctgagggtgtaggggagacaggatgggctggtgctggggacatttagtgtcctggaggcctggctgagttcgggagccagaaggacatgagccctgtcccttctgcatttctgtggtttctggcaggagtaaggggaaatgcctacccttatctcatctctacccccaactgaaggaaatcctctcttcctctcctagatgttccacagaacccaacaactggtatctttccaggagatggctcaggtaggaaggagcctccccgcctggggctgttactgacattgagtctgtgtcaggtttggtcagatctggactttcagagtcaaatgttcagaggcaaggcctgcagttagacacgggtagacatcaggcaccttggaaaaggatatttggggatgactagcaacttcccccttgcccatccaaataatgctctttgtctccctcctgtctctgaatgtcttggggtattttatttttaattgatatgtaataatagtacatatttatggatggcatagtgatgtttccatactaataatgtatagtaatcagatcagggtaatagcatatccatcatcttgaacatttattatttcattgttgttgggaacattcaatatcccctttctagctatttgaagctatctattattgttaagcatagtcatcctacagtggtatagaacaccagaacttattcttcctttccaggtgtaatctagtatcctttaacaaatctctctccttatcattgttcccctaaccttcccagcccttattattctctgttctactttttacttctatgaaatcaacttcttgtagcttccacttatgagtgagaacatgtggtattcaactttctgttcctagcttatttcatttaacataatgtcctctagttcaatctatgttatagtgaataacaagatttcattattttttatggctgaatgataatccattgtgtatatacgccacatttcctttatttattcatctgttgttggacacttaggtttatttcatatcttcctattgtggataatgctgcaataaacattgaggtgcagacgtttcttcaatatactgatttcctttcctttctataaatgcccagtagtggggttgctggatcatatggtagttctatttgtagttttttgagaaacttccatactcttctccatagtggttatactagtttacattctggtcaaaagtatataagagttccctcttctctacatcctcaccatcatttgttaattttcatcttttttttatcatagtcctcccaactggggtgatgttacctcattgtggttttgatttgcatttccctggtgattggtgacgttgagcatttttcatatacacttgttggccatctgtatatcttttcttgagaaatgtctactcagataatttgcccatttttaaatgagattgggtttctttgccattgagatgtatgagttcctcgtatgttctggatatgaatcacttgtcagatgaatagctgacaaatattttctcctattctgtaggttgccttttcactctgttggttgtttcctttctgcatagaagctttttagcttgatatcatctcatttatttacttttgcttttgttgcttgtgctagtgaggtcttactcataaaatatttttccagaccaatgtcctaaagcatttcccctatgtttttttctagtattttttaaattttgtgtcttatattcaggtctttgatccattttgaattgatttttgtataggacgagaggtgtgagtctaatgtcattcttctgcatatggcaccagttttcccagcatcatttattaaagaaactgctctttcctcaatgagtgttcttcatgcatttgtcaaaattcagttggctgtagatcgtggattaatttcggtgttctctattatgtattattggtgtatgtatctgcttttatgccaatatcatgctgttttggttactacagctttgtagttttgaaatctttaaatttttgaaattttgaaattttctagttttgaaattttgaaatcttgtagtgtgatacctccagctttgttcttttttgcttgggattgctttgaccattcaggctatttttagttccatatgaattttaagattgtttcctctaattctgtgaagaattacattgatattttgatagagccaggtttgaatctgtagatttctttgggtagtataatcattttagcaatattaattcatctgatgagtaaggaatgtctttccatttgtttgtatcctcttcagtttatttcctcagtgttttgtagtttttcttattaaggcttgtcacctccttggttaaatttattcctaggtatacttcattctcttatagctattgtaaatgtgattgccttcctgatttattttcagctaattcattgtgtgtagaaatgctactgatttttgtatattgattttgcatcctgcaaatttactaaattcatttatcagttctgagagttttattgttagagtctttaggtttttgttttgttttgttttgttttgttttgtttttgagatggaatttcaccatgttggccaagctggtcttgatctcctggcctcaagcaatctgcccactttggcctcctaaagtgctggaattacaggcatgagccaccacgcctggccaagtctttaggtttttgtatgttatttgcagagacaatttgacttccgcctttccagtttggatggtttttatttctttctcttgcctaattgctctggctaggactttcagtactatgtaaaataagagtcataacagtggacatccagttcctagaggaaaagatttcagcttttctccattcagtatgatgttagccatgggtttgtcatatatggccttttttgtgttgaggtactttccttctatacctaatttattgagagtttctatcatgaaacaatattgaattttaacacatgctttttattctgcaactatttaggtgatcatacggtttatgtccttcattctgttgacatatgtataacatttattgatttgcatatgttgaatcattcttgcctttctgggattaatcccactttatcatggtatgttatctttttgatgtattgttggatttgatttgctactattttgttgaatatttttgcatctatgttcatcagggatattggcctctagttttctttttttattgtctcctttctgattttggtgtcatggttatgctggccttgtagaatgagttaggaagagttgcctccacttcaattttttggaatagtttgagaagagttggcataatttttttttctttaaaggttcagtaaagttcagcactgaagccatccagccctggaattttctttgttggggggccttttattattcattcaatctcattacttgttgtttgtctgctgaagttttctataccttcttgattcaatctcggtagattatatgtgtccaggaacttatccatttcttctagactttcaaatttgttggcatattgttcatagtagtgtctaagatcctgtgtatttctgtggtaaccattgtgacatcttcttttttatttatgattttattaatttttatgtcttctgtctctttcttagtttagctaatgattgtcaattttatttatttttccaaaaagcgaacttgttcattgatttttttttaatttcatttatttctgctctgatctttatgatttctttcattgtgctgattttggatttggtttgttcttgctttctagtttcttgaaatgcacagttaaatggtttacttgaaatttgtctaattgtttgatgtaggcatttatttctctcaagttgtctcttaaaactgtttttgctgtgtcccataggttttggtatattttatttctatttttatttattttgagaaatttttaaatatcattcttaatttcttccttcactattggtcatttagaatcattttgtttcatttctgtgtatttgtatagtttgcatgtttcccttggtattgatttttagttttattcaattgtagtcaaataagatacttgatacattttggtttttaaaaatttttggcacttgttttgtgttctaacatatggtcgatccttgggaatgttgcacatgctgatgaaaccatgtgtattctgcagctgtcggttgaaatgttctgtaaatatcttaggttcatttggtatatggtgcagtttaaatccaacgtttatttgttaatcttgtctagatgatttgttcaatgctgagagtggggcgttgaagtcctcaactattattgtattggagtctatctctccctttatatctaataatatttgctttacatatctgggtgctctggtgttgtgtgcatatgtatttacagttgttatattatagtgctgaactgacccctttataataatataatgtccttctttgtctctttacagcttttgacttgtagtccgttttgtctgagataagtatagctattcctgcttcctttcatttccacttgggtagaatatctttttccatctcttccttttcagtctatgtgtgtcttctaggtgagataagtttcttgtaagcagtatatagctgtgttggtagaagggctgaggcagggcttgcttgtctgacataatgtaaaagagtcttggaacatgtcctgggtccagggtctcaaacccctcgtggcctatggaacaccaagctctgtgcctaagggtggaaggctgccctgccacactgcaatctaagcccagggcataaaacccctcgtggcttggaaagaatccagggctctgggcataaaacccctcatagcctctggaatgtgtccagacttgctggccccttgctccttgctctcccaggatcataaattgattgtatcttgagtgaaaagaacttgttctccattatttcaagtagcagagcatatgctaaaccgtcacagctatgcttgatgcaccgctacctttctaccccaaagtcctcacgttctcacttgtctatccccacttctgcacgtcctcaccacctgcttctttgtttgattaccaataaatagtgtgggctcccagagctcggggccttcacagcctccatactagcgtcggccccctggactcactttatgtactattaacttgtcttgtctcattcctttgactccgctggacttcgtggcccccacggcctagtgttggatctgatcaccccaacaagctgagtctagattttcttttcattcattcaggcagtccatatattttaaatgggacaatttaatccatttacatacacattattattaataggttattttcatttcattgattgttttctgattgttttatatattcctggttccttacttcccctcttattgtttctttttgtggttggctgatgttttttttttttttgtagtgataagatttgattcctttctctttcttctttgtgtatgggctgtcagtgagttttaagttcacgtgtttttgccttttcacttccagatgtaagactcccttgagcatttcttttctttttcttctcttatttatttttattattttttttttgagaaagtgtctcactctgtcgcccaggcaggagtgcagtggcatgatcacggctcactatagtctcgacctcctgggcttaagcaatcttcctgccttaacctcccaagtagctgggactacaggcatgtgccaccacgcccagctaatttttgtgtttcttgtagaggtagggtgttgccatttgcctaagctggtctcaaattaaagagctcaagtggtccacctgcctgccttcacctcccaatgtgctgggattataggcatgagccacactgtgcctggccccttgagcatttcttgtaaggccagtctaagagtgattagaattcccttagtttttgcttatctatgaaatattttatttctccttcttttctgaaagatagcttttctgggtatagtatttttgactgttaagttttttatctttcagtactttgagtatgtcatcccattctatcctggcctatataatgttactgctgagaaactcactgttagtctaataaggataatcctatatgtgactagatacttttaccttgctgtttttacaattctttacttgacttttgacaatttggcataatgagctttggagaggacttgcttgggttgaatattttgagagtactttgagcttcctggacctggatgtccttctagttcccaaggcttgggaagttttcacctattactggattaaatatgttttctacaccttttccattctcttctcctcctggaaataccataatgtgaatatttgcttgattgtgtcccatgagtcctgtaggtttccttcgttctattttattctcttatttttacctgcctgtgttatttcagaagatctgtcttcaagttcagaaattattttttcttcttgacctagcctgttgttgaagctctcgattgcggttttttatttcatttattgagttctcagctgtaggagttctgctttgttcttttatataatatctatctctctgttaaatttctctttcaagtcatgaattaaaacaatgggacacaggtgcccaactacttggctgacctgggggcatatctgctggaggtgccaacatggctgttttgcagggctgagatgaagctgaatgactcttggctggcctaggtgtgtttttgccaggagtagcactcagagctttatctagggtttgggatgtgagtgtaagactgctcagctggcctagggggtgtaccagccagtggtagcccatggggctgtttctcaggcctggaatgcaagcacattctgcctggggtcatgtctaaaagggttggctcacaaggctgtttctcaggccctaattgtgggagagtggcctttgggcaggccagagtcatgtccacagaaggcgtctgggcaccgtaaggctgtttctcagagcctgtgtgtgagcacataaccactaccccagcctggggatgtatcaactctttgttggctcagaggtctctcccattcaggtgagcatgcacagtagtttggccaactcaattgtgtgttcgccctgagtgggactataagacctttcctccagctggaagtacgggcagcaggggttggtttctctgctgttcagggccagagtcccagccaatcctgggcccaggctccatgcagctctaattgtggtattcagccactactgcaggtttagtggaatgaagatgcacaatgataaagaggtgcatgccactggcccccagaggagggtgcactccagagatggctgtggtctcaagatggttctgtgttgtagcagcttgcccgcaggggctggttagggagttgggagtgcacaccaaatgctccatgcagctgtgtgaattcctggcagctcttccaactgtgctcagagcttgtgaggactgtaagattaacctgtagtaaggaatgtaggtatctgcagtggcactggaggttggttggattcctctgcttatcatttccctacaaggggaaatccttcctgtctctgggacaaaccaatctgggctggggagatggagctgcaaagcccgggtgcctccatgctgccctcctgggtttccaattaccacaggtaactctccactcccttgctgcactacactactctcccttcgacactccactcaaatctttgctgtggtttattcattgccttggtcctttcttgtctggtgacacgggggaggatgagctccaggcacctccggtgagccattttgctccaatgggggcatttttttttaataggttttatttttcagagtagtttttgtttcacagcaaaattgagtggaatcttctagtcgctgatcatcttgggagcatttataaatgaaccttatttttcatgaagaaattgagcagaagatactaagacttcccgtatgccctctacccttacacatagtttccccggccatcagcatcccccatcagagtggtacatttgttacagtcaataaaactacattgacatatcattgtcacctgaagcccatagtttacattaaagttcactcttggtgttgtacattttacaggctttttaaaaatgtataatgacatgaatccaccatgagagtatcatatagaatagtcacacttccctaaaaatctctttagggcatttttttctactgtccatacctcaacccttagccctggcctctgtccaaagaccagtgctctctccactgccctattccaattaataatggcatctggcacctcagtggacagtgagcccagtgagagcaggaacagttccctcagtagtggttatcaaactgttaacaatgatgctcagagacacgcccctgactctgagtgttgggacctagaaggcacagccaggcaggtccaggagaactgtctgggtctaagaaggtctgagaaccacctccctgccccaccctgcttccaggccctttttaaggccaaaaggaccacctttgaccctaagtgatggggccagtgggaagaaagaagagacaaggcctatcagcattccagtgctttctctctctctcatccaagaggctcagagcttcacagtccttcaggggctatgtctgaggttcatttcagaaagacccagggtggagaggaacctgagtcctaggagagatgatgttttgtgcaccagagagagagggtgggacaagaggtgtcaggtgcactgtgtacttcatctcatggtcgtggtcaatattgatgtctatgatgggtgggaagatctaggagctaaaccccattttggaggtgaagtcacccctctctacatgctggagaggaggatacacatacctgtttatctagattagaattcaccccaaatcttttttgtctgcagggaaacaagagaccagagcaggagtggttcatggggccattggaggagctggtgttacagccctgctcgctctttgtctctgcctcatcttcttcatgtgagcattttctctgggtcaggcatgggccagaggtgaagaggatggacctggtgtagaagggtcctggaggggctgtgagggctggagaaagggcagggggtgtgatgatgtacagaatccagcctgtggccactgggataggcgtgggtctattccagggccctgatctcagatgtccaaggagtgggaggtagagggagaccttgtgactaagtcttgtttgagggctcctggattaatcccaccctttacctgccaaagtccctcattccaggctcataacaatggccccacagcctgagaaaaccaggctcaaagaccctggtgtctcccatcagagtgaagacccacaggaggaaagcagccaggacagcagtgggcaggaatgacacccaccctaccacagggtcagcctccccggtgagtgatggggcatcctggcatccagtctgtcctgcagacacctcctcccaatgtggcccaccgtcatgccccattcagcatttccagaactgagcttattgtctttcctcctgtttaacagtgtaggttttaatatttttcaggtacgttgaggccaacagatcaggagatgatggccattgaaaagatagtttcttggccgggcacagtgtttcacacctgcaatcccagcacctttggaggccaaggcgggcggatcacgaggtcaggagattgagactatcctggctaacatggtgaaaccccgtctctactaaaaatacaaaaaattagccagatgtggtggctggcgcctgtagtcccagctacttgggaggctgaggcaggagaatggtatgaacccgggaggcagagcttgtagtgagccgacatagcaccactgcactccagcctgggtgacagagagagactctgtcccaaaaagaaaaaaaaaatagtttcttattcaccgttcccgagagggcacaccacaccatgcaaggccatatggagaagcaccagggtcagtcaggaagcagagggagcaaggagaaaatgggacaagagccttcactgtggctttcatggaaaagaatgggcaagacagggtaagcaagctaggcaggtttaggattggctacttagaacaatttcagcagactctggggtataggagttgtctctagttgtctggtacatggccctgggtttattaaggaggattgtggtctggagtgtaagagctcaataaaggatccagctggtagtgtgggctttagattgactggtttgcacatgaaaggtgcacttgtatgcaagtcctttattagctctagaaatctactatccttgggaaaggcagtctctcaagggtcagtaatgccccagatgtcaaaacatcagaaacacttggttgacacacccctaaacatacttctcctgatgggttctccatctcgctgatggcactcttgtccccattacccaaccagagacatggccccctcctgtcccagtcctccatctcttcctgtgccagtatgctacgatgcatgtctgagcttcctctgaacacggcttaacacaaccactcctgagccgagagcccctcttactccttattctgctgcagcctcacctcccatttctcctctccagaacattagcatcacctccctaaaaggtcattgtcccatcattcccaagtttgaaatgcactgcttctctacactcctgaaagattggcattccaacaacttggtctggcatttggagcaggaaaaccagagtccccttcagtgctatgctcccccaacattagccactcaatcacctcaagcagggcaagctttctcatctcagaatcattgctgggctgtcccctcctcctcatatgcctaatagctacctgcccaactccagtgcatccttcaagctctgattttttttttaatttatttaactctgactaaagtgaacaccacagtaaagtttttgaacacagggtcaaccagcacccattcattctgaaatatctatataatcccatttgccaattgctctaggtccttgtgccattctgtatttttataaacacacagtttacaaatataaaatattcctcctatcgggggcttaacatttattggggaaagggatgaaaataatgaacaaataagcagtgcaaatatacatgaaataggcatgaaataagtgctatggcaggaaatgaaatgggagaacggattggacagtcctgggggccaaagaatggcctttgggcaaacacctgcagaaagaaagtgagtacagtatgagcagtagagaatcatcaaggaagcagcaagtaccatggctctgaggccggaacacatctgatgttttagagaaacaaagtaggacagtgtggataaggcagagttacgtgttgggggtggagtgtggactgaacaatggtaggtaggaaatgaggttgaagagacataggagctgcaaatattgcaggatcctaagtccatcatagttattgatgcgtttagagcagaagagtgacatgaactgacactcattttagtgggagtcactctggctgctctgtgagaaactctagtatgtagtatagaagagaagataccagaatagaagatacacagataatcaagccaagagatgactgactcagacttagtcccaagtaagaataatgaatctgatgtggagaaattgggttccggatacatcttgaaggtggagtcaacagtatttgcaagtggagtgatggagtgcatgcaaggcatgagcaaagatagctcacgggctctgctcgataagtgtctcagatgtcataggtgggtccacagatatcatagatgtcatggatgtccaagaagaccccatagatgtcatagatgtctatagatgccaatgatgatgttcatgtggttaactcagaattcaaacttaaaaaatcaaattgccaatgattaaaattgccatccttgaaggagaagtctattttcaagtgtatatcaaacattattttggttaccgtaagtttgaggtgcctctgagacatccatgtggagatgacaagtagcaggcaagtgtctggagctcagaagagggctccacgtgggagacacagaggttgggagcttccctatcaacatccaataatcgatgaatctacaaagaagagaaggggtccacagacacagccctggaaccctccagcatttaatgttagggagatgaggtgtaaatggtgagaaagctgagaatgaataggagccaatgaccaggaaattgcaggctgtagtgttggtggccaagggaagatggggcttcatggaggagaggttggtcatttgtttcaaatgctggtaagtctggtaagatggaatctgagaaatggctatttgaatgtagctaagtggtatgacagcaaagcagatctgatttttctgctggaggaagatctcttgactagagagagttcaagagagaatgggaggagaagaggcagaaactgtgagttgaaggactcttttgagaaacactgccccaaatctagaaccaagaaatgggcctgcaccagcaaatggttgtaccctgtagacttgccatttctccagcatctgctcctgtgtctcttatgataccatgttccctgtttgtgtagggctttgcaccacttgaactaactgcattcccatagcttcccctaccacaccatgagctccacaaggaaaagcctgggttttattagacctccatcattctactctctcctgttcatctgcacactgtcacaatttgcaattaccagtctgtttctgtcttcatcccctgcagtactggaaatcacagggcccctgctctgctctgctccctcctgaggatccagtgcccagcacataggaggtcccagagacctgggaccgagttcagggtcaacagatgtgtgactttggaaattacctaagctctctgagacctagtttcctggtctgtaaaatggattaaaataatagatgccaaagatgatgtcagtgtagctgcccagaatttttccacattagtctctgtgagtattcaagagaattgcgaatcaatcaatacgtgctacatgtgttagataatgaataagtagagccttaattaattaacatttgatgaaagaatgaaagagtgaataaatgttctgtcagagtcaaatttacttcattgaccctctttgccttctcctggtccccctcctcactgccctgctctaacccccttctttcctctccataagaaacaccagaagaagtccaagttacatggccccactgaaacctcaagctgttcaggtgccgcccctactgtggagatggatgaggagctgcattatgcttccctcaactttcatgggatgaatccttccaaggacacctccaccgaatactcagaggtcaggacccagtgaggaacccacaagagcatcaggctcagctagaagatccacatcctctacaggtcggggaccaaaggctgattcttggagatttaacaccccacaggcaatgggtttatagacattatgtgagtttcctgctatattaacatcatcttagactttgcaagcagagagtcgtggaatcaaatctgtgctctttcatttgctaagtgtatgatgtcacacaagctccttaaccttccatgtctccattttcttctctgtgaagtaggtataagaagtcctatctcatagggatgctgtgagcattaaataaaggtacacatggaaaacaccagtc

SEQ ID NO: 8 - DNA, 20 bp, Artificial Sequence
  Synthetic Polynucleotide
tgttccacagaacccaacaa

SEQ ID NO: 9 - DNA, 20 bp, Artificial Sequence
  Synthetic Polynucleotide
ggctgtaacaccagctcctc

SEQ ID NO: 10 - DNA, 20 bp, Artificial Sequence
  Synthetic Polynucleotide
ccctgctgtgggcagacttg

SEQ ID NO: 11 - DNA, 21 bp, Artificial Sequence
  Synthetic Polynucleotide
gcaccgaggagtgagtagtcc

SEQ ID NO: 12 - DNA, 20 bp, Artificial Sequence
  Synthetic Polypeptide
tccccacaggggccctggct

SEQ ID NO: 13 - DNA, 20 bp, Artificial Sequence
  Synthetic Polynucleotide
tccccacaggggtcctggct

(19) United States
(12) Patent Application Publication
(10) Pub. No.: US 2019/0203299 A1
(43) Pub. Date: Jul. 4, 2019

(54) BIOMARKERS FOR ANTI-LEUKEMIC THERAPY

(71) Applicant: University of Florida Research Foundation, Incorporated, Gainesville, FL (US)

(72) Inventor: Jatinder Kaur Lamba, Gainesville, FL (US)

(73) Assignee: University of Florida Research Foundation, Incorporated, Gainesville, FL (US)

(21) Appl. No.: 16/091,720

(22) PCT Filed: Apr. 6, 2017
(86) PCT No.: PCT/US17/26369

§ 371 Date: Oct. 5, 2018

Related U.S. Application Data
(60) Provisional application No. 62/319,284, filed on Apr. 6, 2016.
(60) Provisional application No. 62/320,306, filed on Apr. 8, 2016.

(51) Int. Cl. C12Q 1/6886; C07K 16/28; A61K 47/68

(52) U.S. Cl. CPC C12Q 1/6886; C07K 16/2803; C12Q 2600/156; A61K 47/6849; C12Q 2600/106; A61K 47/6803

(57) ABSTRACT

Aspects of the invention relate to detecting single nucleotide polymorphisms for CD33 and determining whether a subject is likely to respond to treatment with agents that selectively bind to CD33 based on the genotype of the subject for one or more CD33 single nucleotide polymorphisms.

This application is a national stage filing under 35 U.S.C. § 371 of international application number PCT/US2017/026369, filed Apr. 6, 2017, and claims the benefit under 35 U.S.C. § 119 of U.S. provisional applications 62/319,284, filed Apr. 6, 2016, and 62/320,306, filed Apr. 8, 2016, the entire contents of each of which is incorporated herein by reference.

### BACKGROUND OF THE INVENTION

### Field of the Invention

Acute myeloid leukemia (AML) is a heterogeneous disease with dismal outcome. While intensive chemotherapy and hematopoietic stem cell transplantation remains the mainstay of current AML therapy, targeted therapies such as monoclonal antibodies and small molecule inhibitors have emerged as promising approaches. Gemtuzumab Ozogamicin (GO) is a humanized anti-CD33 antibody linked with cytotoxin calicheamicin, which targets AML cells, majority of which express CD33 antigen (1). GO was approved in 2000 for treatment of relapsed AML in older patients and since then multiple clinical trials have investigated GO in AML (2). GO was withdrawn from the market due to lack of benefit and high early mortality observed in the SWOG S0106 study (3). Reassessment of the results from that trial as well as several other follow-up studies showed improved outcome in subset of patients (3-8).

### SUMMARY OF THE INVENTION

Described herein are methods to determine CD33 single nucleotide polymorphism (SNP) genotype(s) in a subject with a cancer expressing CD33 and to determine whether the subject is likely or unlikely to benefit from a particular treatment. Methods of treatment are also described.

In some aspects, the disclosure relates to a method of treating a subject with a cancer expressing CD33 comprising: performing an assay to detect the genotype of the subject for the CD33 single-nucleotide polymorphism rs12459419, wherein the genotype may be CC, TC, or TT; and administering a therapeutically effective amount of an agent that selectively binds to CD33 if the subject exhibits a CC genotype for the CD33 single-nucleotide polymorphism rs12459419.

In some embodiments, the agent that selectively binds to CD33 comprises an antibody that selectively binds CD33, or an antigen binding fragment thereof. In some embodiments, the antibody that selectively binds CD33 is a humanized antibody. In some embodiments, the agent that selectively binds to CD33 comprises an antibody that selectively binds CD33, or an antigen binding fragment thereof, conjugated to a toxin. In some embodiments, the agent that selectively binds to CD33 selectively binds to amino acids encoded by exon 2 of CD33. In some embodiments, the agent that selectively binds to CD33 is gemtuzumab ozogamicin (GO), hP67.7, or SGN-33A.

In some embodiments, the subject is a pediatric subject. In some embodiments, the subject is an adult subject. In some embodiments, the subject is treated with chemotherapy within thirty days of the administration of the antibody.

In some embodiments, the assay is performed by DNA sequencing analysis. In some embodiments, the cancer is acute myeloid leukemia (AML), acute lymphoblastic leukemia (ALL), or acute promyelocytic leukemia (APL). In some embodiments, the assay is a hybridization assay. In some embodiments, the subject has one or more of: the presence of blast cells that express CD33 within the hematopoietic system; leukostasis; anemia; leukopenia; neutropenia; thrombocytopenia; chloroma; granulocytic sarcoma; and myeloid sarcoma.

In some aspects, the disclosure provides a method for determining whether a subject with a cancer expressing CD33 is likely to benefit from treatment with an agent that selectively binds to CD33 comprising: providing tissue from a subject who has been diagnosed with the cancer; performing an assay on the tissue, or on a derivative of the tissue, to detect the genotype of the subject for the CD33 single-nucleotide polymorphism rs12459419, wherein the genotype may be CC, TC, or TT; wherein the subject is likely to benefit from treatment with an agent that selectively binds to CD33 if the subject exhibits a CC genotype for the CD33 single-nucleotide polymorphism rs12459419 and the subject is not likely to benefit from treatment with an agent that selectively binds to CD33 if the subject exhibits a TC or TT genotype for the CD33 single-nucleotide polymorphism rs12459419.

In some embodiments, the agent that selectively binds to CD33 comprises an antibody that selectively binds CD33, or an antigen binding fragment thereof. In some embodiments, the agent that selectively binds to CD33 selectively binds to amino acids encoded by exon 2 of CD33. In some embodiments, the agent that selectively binds to CD33 is gemtuzumab ozogamicin (GO), hP67.7, or SGN-33A. In some embodiments, the subject is a pediatric subject. In some embodiments, the subject is an adult subject. In some embodiments, the assay comprises performing DNA sequencing analysis. In some embodiments, the assay comprises contacting a derivative of the tissue with a nucleic acid probe. In some embodiments, the assay is a hybridization assay.

In some embodiments, the cancer is acute myeloid leukemia (AML), acute lymphoblastic leukemia (ALL), or acute promyelocytic leukemia (APL). In some embodiments, the subject has one or more of: the presence of blast cells that express CD33 within the hematopoietic system; leukostasis; anemia; leukopenia; neutropenia; thrombocytopenia; chloroma; granulocytic sarcoma; and myeloid sarcoma.

In some aspects, the disclosure provides a method for detecting a polymorphism, comprising obtaining a biological sample of a subject that has a cancer expressing CD33, performing an assay to detect the genotype of the subject for the CD33 single-nucleotide polymorphism (SNP) rs12459419 controlling expression of exon 2, wherein the genotype may be CC, TC, or TT and wherein the presence of the CC genotype in rs12459419 indicates expression of exon 2 of CD33.

In some embodiments, the assay is a hybridization assay comprising a probe that hybridizes specifically to the CC genotype but not the TC or the TT genotypes. In some embodiments, the hybridization assay further comprises a probe that hybridizes specifically to the TT genotype but not the CC or the TC genotype and a probe that hybridizes specifically to the TC genotype but not the CC or the TT genotypes. In some embodiments, the method comprises detecting specific hybridization of the probes that binds specifically the CD33 single-nucleotide polymorphism rs12459419 to their respective genotype.

In some embodiments, the hybridization assay comprises detecting hybridization of a probe that binds to the a nucleic acid from the biological sample, and detecting a variant nucleic acid of CD33 single-nucleotide polymorphism rs12459419 in the sample when hybridization is detected. In some embodiments, the method further comprises performing a hybridization assay with the probes and a control genotype.

In some embodiments, the assay is a genomic sequencing assay. In some embodiments, the assay is a DNA sequencing, RNA sequencing, primer extension, enzyme-based, restriction fragment length polymorphism, PCR-based, PCR-RFLP, allele-specific PCR, flap endonuclease, 5′-nuclease, oligonucleotide ligation, SNPlex, surveyor nuclease, dynamic allele-specific hybridization, molecular beacons, or SNP microarray assay. In some embodiments, the genomic assay comprises direct sequencing of a nucleic acid containing polymorphism rs12459419, and detecting the presence of the CC, TC or TT genotype. In some embodiments, the nucleic acid is DNA, genomic DNA, RNA, cDNA, hnRNA or mRNA.

In some embodiments, the subject has one or more of: the presence of blast cells that express CD33 within the hematopoietic system; leukostasis; anemia; leukopenia; neutropenia; thrombocytopenia; chloroma; granulocytic sarcoma; and myeloid sarcoma.

In some embodiments, the probe comprises a nucleotide sequence complementary to a sequence listed within Table 1. The probe can comprise additional nucleotides. In some embodiments, the probe comprises a nucleotide sequence complementary to nucleotides of SEQ ID NO: 1. In some embodiments, the cancer is acute myeloid leukemia (AML), acute lymphoblastic leukemia (ALL), or acute promyelocytic leukemia (APL).

In some aspects, the disclosure provides a method for detecting a polymorphism, comprising obtaining a biological sample of a subject that has a cancer expressing CD33, performing an assay to detect the presence of amino acids encoded by exon 2 of CD33, wherein the presence of amino acids encoded by exon 2 of CD33 indicates expression of exon 2 of CD33 and presence of a CC genotype in single-nucleotide polymorphism rs12459419 of CD33. In some embodiments, the assay is an immunoassay. In some embodiments, the assay is a protein sequencing assay.

In some aspects, the disclosure provides a kit comprising an agent that selectively binds to CD33 and instructions indicating the use of the agent to treat a subject if the genotype of the subject for the CD33 single-nucleotide polymorphism rs12459419 is CC.

In some embodiments, the agent that selectively binds to CD33 comprises an antibody that selectively binds CD33, or an antigen binding fragment thereof. In some embodiments, the antibody that selectively binds CD33 is a humanized antibody. In some embodiments, the agent that selectively binds to CD33 comprises an antibody that selectively binds CD33, or an antigen binding fragment thereof, conjugated to a toxin. In some embodiments, the agent that selectively binds to CD33 selectively binds to exon 2 of CD33. In some embodiments, the agent that selectively binds to CD33 is gemtuzumab ozogamicin. In some embodiments, the subject is a pediatric subject. In some embodiments, the subject is an adult subject.

In some aspects, the disclosure provides a method for determining a CD33SNP_Score for a subject comprising: determining genotype scores of the subject for the CD33 single-nucleotide polymorphisms (SNPs) rs12459419 or rs3865444, rs1803254, rs35112940, and rs2455069, wherein the genotype score for a single nucleotide polymorphism (SNP) with two wild-type alleles is 0, the genotype score separately for each of the SNPs rs12459419, rs3865444, rs1803254, and rs35112940 with one wild-type allele and one variant allele for is −1, the genotype score for the SNP rs2455069 with one wild-type allele and one variant allele is 1, the genotype score separately for each of the SNPs rs12459419, rs3865444, rs1803254, and rs35112940 with two variant alleles is −2, and the genotype score for the SNP rs2455069 with two variant alleles is 2, and adding the genotype scores to yield the CD33SNP_Score.

In some embodiments, the genotype of each SNP is determined by an assay. In some embodiments, the assay is performed by DNA sequencing analysis. In some embodiments, the assay is a hybridization assay. In some embodiments, the subject is a pediatric subject. In some embodiments, the subject is an adult subject.

In some aspects, the disclosure provides a method for determining a CD33SNP_Score for a subject comprising adding genotype scores of the subject for the CD33 single-nucleotide polymorphisms rs12459419 or rs3865444, rs1803254, rs35112940, and rs2455069 to yield the CD33SNP_Score, wherein the genotype score for a single nucleotide polymorphism (SNP) with two wild-type alleles is 0, the genotype score separately for each of the SNPs rs12459419, rs3865444, rs1803254, and rs35112940 with one wild-type allele and one variant allele for is −1, the genotype score for the SNP rs2455069 with one wild-type allele and one variant allele is 1, the genotype score separately for each of the SNPs rs12459419, rs3865444, rs1803254, and rs35112940 with two variant alleles is −2, and the genotype score for the SNP rs2455069 with two variant alleles is 2.

In some embodiments, the genotype of each SNP is determined by an assay. In some embodiments, the assay is performed by DNA sequencing analysis. In some embodiments, the assay is a hybridization assay. In some embodiments, the subject is a pediatric subject. In some embodiments, the subject is an adult subject.

In some aspects, the disclosure provides a method for determining whether a subject with cancer is likely to benefit from treatment with an agent that selectively binds to CD33 comprising: determining a CD33SNP_Score for the subject, wherein tissue is provided from the subject who has been diagnosed with the cancer; an assay is performed on the tissue, or on a derivative of the tissue, to detect the genotype of the subject for the CD33 single-nucleotide polymorphisms rs12459419 or rs3865444, rs1803254, rs35112940, and rs2455069, wherein the wild-type, single variant, or double variant genotype, respectively may be: CC, TC, or TT for rs12459419, CC, CA, or AA for rs3865444, GG, CG, or CC for rs1803254, GG, AG, or AA for rs35112940, or AA, AG, or GG for rs2455069, and wherein the subject is likely to benefit from treatment with an agent that selectively binds amino acids encoded by exon 2 of CD33 if the CD33SNP_Score for the subject is greater than or equal to zero.

In some embodiments, the assay is performed by DNA sequencing analysis. In some embodiments, the assay is a hybridization assay. In some embodiments, the subject is a pediatric subject. In some embodiments, the subject is an adult subject. In some embodiments, the cancer is acute myeloid leukemia (AML), acute lymphoblastic leukemia (ALL), or acute promyelocytic leukemia (APL). In some embodiments, the tissue comprises CD33 expressing cells, e.g., blast cells comprising CD33.

In some aspects, the disclosure provides a method for determining whether a subject with cancer expressing CD33 is likely to benefit from treatment with an agent that selectively binds to CD33 comprising: providing tissue from a subject who has been diagnosed with the cancer; performing an assay on the tissue, or on a derivative of the tissue, to detect the CD33 single-nucleotide polymorphism genotype of the subject for the CD33 single-nucleotide polymorphism rs12459419 or the CD33 single-nucleotide polymorphism rs3865444, and determining the single-nucleotide polymorphism genotype score, wherein the genotype score may be 0, −1, or −2, wherein a score of 0 indicates that the subject is likely to benefit from treatment.

In some embodiments, the assay is performed by DNA sequencing analysis. In some embodiments, the assay is a hybridization assay. In some embodiments, the subject is a pediatric subject. In some embodiments, the subject is an adult subject. In some embodiments, the cancer is acute myeloid leukemia (AML), acute lymphoblastic leukemia (ALL), or acute promyelocytic leukemia (APL). In some embodiments, the tissue comprises CD33 expressing cells, e.g., blast cells comprising CD33.

In some aspects, the disclosure provides a method of treating a subject with a cancer expressing CD33 comprising performing an assay to detect the genotype of the subject for the CD33 single-nucleotide polymorphism rs3865444, wherein the genotype may be CC, CA, or AA; and administering a therapeutically effective amount of an agent that selectively binds to CD33 if the subject exhibits a GG genotype for the CD33 single-nucleotide polymorphism rs3865444.

In some aspects, the disclosure provides a method of treating a subject with a cancer expressing CD33 comprising performing an assay to detect the genotype of the subject for any one of the CD33 single-nucleotide polymorphisms rs1354106, rs3852865, and rs12985029, wherein the genotype may be wild-type, heterozygous variant, or homozygous variant; and administering a therapeutically effective amount of an agent that selectively binds to CD33 if the subject exhibits a wild-type genotype for the CD33 single-nucleotide polymorphism rs1354106, rs3852865, or rs12985029.

In some embodiments, the agent that selectively binds to CD33 comprises an antibody that selectively binds CD33, or an antigen binding fragment thereof. In some embodiments, the antibody that selectively binds CD33 is a humanized antibody. In some embodiments, the agent that selectively binds to CD33 comprises an antibody that selectively binds CD33, or an antigen binding fragment thereof, conjugated to a toxin. In some embodiments, the agent that selectively binds to CD33 selectively binds to amino acids encoded by exon 2 of CD33. In some embodiments, the agent that selectively binds to CD33 is gemtuzumab ozogamicin, hP67.7, or SGN-33A.

In some embodiments, the subject is a pediatric subject. In some embodiments, the subject is an adult subject. In some embodiments, the subject is treated with chemotherapy within thirty days of the administration of the antibody. In some embodiments, the assay is performed by DNA sequencing analysis. In some embodiments, the assay is a hybridization assay. In some embodiments, the cancer is acute myeloid leukemia (AML), acute lymphoblastic leukemia (ALL), or acute promyelocytic leukemia (APL).

In some aspects, the disclosure provides a method for determining whether a subject with a cancer expressing CD33 is likely to benefit from treatment with an agent that selectively binds to CD33 comprising providing tissue from a subject who has been diagnosed with the cancer; performing an assay on the tissue, or on a derivative of the tissue, to detect the genotype of the subject for the CD33 single-nucleotide polymorphism rs3865444, wherein the genotype may be CC, CA or AA; wherein the subject is likely to benefit from treatment with an agent that selectively binds to CD33 if the subject exhibits a CC genotype for the CD33 single-nucleotide polymorphism rs3865444 and the subject is not likely to benefit from treatment with an agent that selectively binds to CD33 if the subject exhibits a CA or AA genotype for the CD33 single-nucleotide polymorphism rs3865444.

In some embodiments, the agent that selectively binds to CD33 comprises an antibody that selectively binds CD33, or an antigen binding fragment thereof. In some embodiments, the agent that selectively binds to CD33 selectively binds to exon 2 of CD33. In some embodiments, the agent that selectively binds to CD33 is gemtuzumab ozogamicin, hP67.7, or SGN-33A. In some embodiments, the subject is a pediatric subject. In some embodiments, the subject is an adult subject. In some embodiments, the assay comprises performing DNA sequencing analysis. In some embodiments, the assay comprises contacting a derivative of the tissue with a nucleic acid probe. In some embodiments, the assay is a hybridization assay. In some embodiments, the cancer is acute myeloid leukemia (AML), acute lymphoblastic leukemia (ALL), or acute promyelocytic leukemia (APL).

In some aspects, the disclosure provides a method for detecting a polymorphism, comprising obtaining a biological sample of a subject that has a cancer expressing CD33 and performing an assay to detect the genotype of the subject for the CD33 single-nucleotide polymorphism (SNP) rs3865444 controlling expression of exon 2, wherein the genotype may be CC, CA, or AA and wherein the presence of the CC genotype in rs3865444 indicates expression of exon 2 of CD33. In some embodiments, the assay is a hybridization assay comprising a probe that hybridizes specifically to the CC genotype but not the CA or the AA genotypes. In some embodiments, the hybridization assay further comprises a probe that hybridizes specifically to the AA genotype but not the CC or the CA genotype and a probe that hybridizes specifically to the CA genotype but not the CC or the AA genotypes. In some embodiments, the method comprises detecting specific hybridization of the probes that bind specifically the CD33 single-nucleotide polymorphism rs3865444 to their respective genotype.

In some embodiments, the hybridization assay comprises detecting hybridization of a probe that binds to the a nucleic acid from the biological sample, and detecting a variant nucleic acid of CD33 single-nucleotide polymorphism rs3865444 in the sample when hybridization is detected. In some embodiments, the method further comprises performing a hybridization assay with the probes and a control genotype.

In some embodiments, the assay is a genomic sequencing assay. In some embodiments, the assay is a DNA sequencing, RNA sequencing, primer extension, enzyme-based, restriction fragment length polymorphism, PCR-based, PCR-RFLP, allele-specific PCR, flap endonuclease, 5′-nuclease, oligonucleotide ligation, SNPlex, surveyor nuclease, dynamic allele-specific hybridization, molecular beacons, or SNP microarray assay.

In some embodiments, the genomic assay comprises direct sequencing of a nucleic acid containing polymorphism rs3865444, and detecting the presence of the CC, CA or AA genotype. In some embodiments, the nucleic acid is DNA, genomic DNA, RNA, cDNA, hnRNA or mRNA.

In some embodiments, the subject has one or more of: the presence of blast cells that express CD33 within the hematopoietic system; leukostasis; anemia; leukopenia; neutropenia; thrombocytopenia; chloroma; granulocytic sarcoma; and myeloid sarcoma. In some embodiments, the probe comprises a nucleotide sequence complementary to a sequence listed within Table 1. The probe can comprise additional nucleotides. In some embodiments, the probe comprises a nucleotide sequence complementary to nucleotides of SEQ ID NO:2. In some embodiments, the cancer is acute myeloid leukemia (AML), acute lymphoblastic leukemia (ALL), or acute promyelocytic leukemia (APL).

In some aspects, the disclosure provides a kit comprising an agent that selectively binds to CD33 and instructions indicating the use of the agent to treat a subject if the genotype of the subject for the CD33 single-nucleotide polymorphism rs3865444 is CC. In some embodiments, the agent that selectively binds to CD33 comprises an antibody that selectively binds CD33, or an antigen binding fragment thereof. In some embodiments, the antibody that selectively binds CD33 is a humanized antibody. In some embodiments, the agent that selectively binds to CD33 comprises an antibody that selectively binds CD33, or an antigen binding fragment thereof, conjugated to a toxin. In some embodiments, the agent that selectively binds to CD33 selectively binds to exon 2 of CD33. In some embodiments, the agent that selectively binds to CD33 is gemtuzumab ozogamicin (GO). In some embodiments, the subject is a pediatric subject. In some embodiments, the subject is an adult subject.

Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth in the following description or illustrated in the drawings. The invention is capable of other embodiments and of being practiced or of being carried out in various ways.

### BRIEF DESCRIPTION OF DRAWINGS

The accompanying drawings are not intended to be drawn to scale. The figures are illustrative only and are not required for enablement of the disclosure. For purposes of clarity, not every component may be labeled in every drawing. In the drawings:

FIG. 1 shows differences in risk of relapse (RR) and in disease free survival (DFS) from end of course 1 between GO vs No-GO arm associated with rs12459419 genotypes.

FIG. 2 shows risk of relapse from end of course 1 in GO vs. No-GO arm based on different genotype groups for each of rs1803254, rs35112940, and rs2455069.

FIG. 3 shows association of CD33 SNPs with outcome within GO or No-GO arm. For SNP rs35112940, presence of AG or AA genotype was associated with higher risk of relapse in GO arm but not in No-GO arm.

FIG. 4 shows association of rs12459419, rs1803254, rs35112940, and rs2455069 with CD33 expression determined as mean fluorescence intensity in diagnostic leukemic blasts obtained from de novo AML patients from AAML0531 clinical trial. CD33 levels were determined in the diagnostic leukemic blast by multiparameter flow cytometry. Y-axis represents Log 10 CD33 mean fluorescence intensity (MFI) and X-axis represents SNP genotype. Plots show medians as a line between boxes representing the first and third quartiles; the whiskers represent the range after excluding the outliers. The outliers are defined as data points that fall outside of the first and third quartiles by more than 1.5 times the interquartile range. Circles that are outside of the whiskers represent outliers.

FIG. 5 shows association of rs12459419 SNP with CD33 intensity in different risk groups as well as FLT-3 status in AML patients.

FIG. 6A shows that rs12459419 C>T alters binding to an exonic splicing enhancer (ESE) protein.

FIG. 6B shows real-time PCR results from assay using isoform specific primers of CD33-D2 spliced isoform and rs12459419 genotype.

FIG. 6C shows RNA-seq results of CD33-D2 spliced isoform and rs12459419.

FIG. 7 shows that the rs12459419 T allele is associated with lower expression of exon 2 as reflected by exon2/exon 4 ratio.

FIG. 8A shows the association of CD33SNP_Score with CD33 intensity. CD33SNP_Score was created for each patient using genotype information from four SNPs: rs12459419, rs1803254, rs35112940, and rs2455069.

FIG. 8B shows a dichotomized CD33SNP_Score with CD33 intensity. Dichotomized CD33SNP_Score can be ≥0 or <0.

FIG. 9 shows association of dichotomized CD33SNP_Scores, ≥0 and <0 with response by arm for following clinical outcomes: disease-free survival (DFS) from end of induction 1 by arm in different CD33SNP_Score groups; risk of relapse (RR) from end of induction 1 by arm in different CD33SNP_Score groups.

### DETAILED DESCRIPTION

The following detailed description is made by way of illustration of certain aspects of the disclosure. It is to be understood that other aspects are contemplated and may be made without departing from the scope or spirit of the present disclosure. The following detailed description, therefore, is not to be taken in a limiting sense. Scientific and technical terms used herein have meanings commonly used in the art unless otherwise specified. The definitions provided herein are to facilitate understanding of certain terms used frequently herein and are not meant to limit the scope of the present disclosure. The singular forms “a”, “an”, and “the” encompass the plural, unless the content clearly dictates otherwise. The term “or” is generally employed in its sense including “and/or” unless the content clearly dictates otherwise.

Aspects of the disclosure relate to treating cancers that express CD33. For example, a subject with acute myeloid leukemia, in which the blast cells of acute myeloid leukemia express CD33, may be treated as disclosed herein.

CD33 and Cancers Expressing CD33.

CD33 or sialic acid binding Ig-like lectin 3 (SIGLEC-3, Siglec-3) is a transmembrane receptor expressed on cells of myeloid lineage that binds sialic acids. It is a member of the SIGLEC family of lectins. The extracellular portion of the CD33 receptor contains two immunoglobulin domains—one IgV and one IgC2 domain and the intracellular portion contains immunoreceptor tyrosine-based inhibitory motifs (ITIMs) that are implicated in inhibition of cellular activity. CD33 is found on cells of myeloid lineage and can also be found on some lymphoid cells. CD33 is expressed on blast cells of acute myeloid leukemia (AML) and detected on blasts of 85-90 percent of subjects presenting with AML.

Embodiments of the disclosure relate to treating a subject having a cancer expressing CD33. Cancers that express CD33 include hematopoietic cancers, e.g., acute myeloid leukemia (AML), acute lymphoblastic leukemia (ALL), and acute promyelocytic leukemia (APL). In some embodiments, the subject has AML, ALL, or APL.

Agents that Selectively Bind to or Target CD33.

Aspects of the disclosure relate to administering agents that selectively bind CD33. An agent that selectively binds to CD33 without limitation can be, e.g., an antibody or an antigen-binding fragment thereof, a protein or peptide, a small molecule, or a nucleic acid. An agent that selectively binds to CD33 can bind to nucleic acids or amino acids of the CD33 sequence. An agent that selectively binds CD33 can bind to any region of CD33. In some embodiments, an agent that selectively binds CD33 can bind to exon 2 of CD33. In some embodiments, an agent that selectively binds CD33 can bind to amino acids encoded by exon 2 of CD33. In some embodiments, the agent is an antibody that selectively binds human myeloid lineage cells that express CD33, e.g., human blast cells. In some embodiments, the agent, such as an antibody, selectively binds to the IgV domain of CD33. The IgV domain of CD33 is recognized by the antibody-drug conjugate, gemtuzumab ozogamicin (GO; Mylotarg®; Pfizer/Wyeth-Ayerst Laboratories) and by the hP67.7 antibody.

In some embodiments, the agent that selectively binds CD33 is gemtuzumab ozogamicin (GO). GO is a recombinant, humanized anti-CD33 monoclonal antibody (IgG4 K antibody hP67.6) linked with (covalently attached to) the cytotoxic antitumor antibiotic calicheamicin (N-acetyl-γ-calicheamicin) via a bifunctional linker (4-(4-acetylphenoxy) butanoic acid). GO targets AML blast cells, the majority of which express a CD33 antigen. In some embodiments, GO is used to treat the subject. In some embodiments, GO is used in a therapeutically effective amount. In some embodiments, a subject is also treated with chemotherapy.

CD33 is also a target of the anti-CD33 immunotoxin Vadastuximab talirine (SGN-CD33A) (Seattle Genetics). SGN-CD33A is an antibody-drug conjugate that may reduce multidrug resistance observed in response to treatment with GO. In some embodiments, SGN-CD33A is used to treat the subject. In some embodiments, GO and SGN-CD33A are used in combination to treat the subject. In some embodiments, the subject is also treated with chemotherapy.

In some embodiments, one or more other antibodies that selectively bind CD33, or antigen binding fragments thereof, may be used to treat the subject.

In some embodiments, an antibody or an antigen binding fragment thereof that selectively binds to CD33 is linked to a toxin to target CD33 expressing cancer cells in a subject. Any antibody that selectively binds CD33 may be used.

Isolated.

In some embodiments, the antibodies and other therapeutic molecules used herein are isolated. Isolated means, in the context of an antibody or other biologic, the antibody or other biologic has been removed from its natural milieu or has been altered from its natural state. As such, isolated does not necessarily reflect the extent to which the molecule has been removed from its natural milieu or has been altered from its natural state. However, it will be understood that an antibody or other biologic that has been purified to some degree and to an extent to which it can be used for its intended therapeutic purpose is “isolated”.

Antibody.

In some embodiments, the methods herein employ antibodies. The term antibody is used in the broadest sense and specifically includes, for example, single monoclonal antibodies, antibody compositions with polyepitopic specificity, single chain antibodies, and antigen-binding fragments of antibodies. An antibody may include an immunoglobulin constant domain from any immunoglobulin, such as IgG1, IgG2, IgG3, or IgG4 subtypes, IgA (including IgA1 and IgA2), IgE, IgD, or IgM.

In some embodiments, the methods herein employ antigen-binding fragments. As used herein, an antigen-binding fragment refers to a portion of an intact antibody that binds antigen. Examples of antibody fragments include Fab, Fab′, F (ab′)₂, and Fv fragments; diabodies; linear antibodies (Zapata et al., Protein Eng. 8 (10): 1057-1062 [1995]); and single-chain antibody molecules. Fv is the minimum antibody fragment containing a complete antigen-recognition binding site. This region consists of a dimer of one heavy- and one light-chain variable domain in tight, non-covalent association. In this configuration the three CDRs of each variable domain interact to define an antigen-binding site on the surface of the V_{H}-V_{L} dimer. Collectively, the six CDRs confer antigen-binding specificity to the antibody. The Fab fragment also contains the constant domain of the light chain and the first constant domain (CH1) of the heavy chain. Fab fragments differ from Fab′ fragments by the addition of a few residues at the carboxy terminus of the heavy chain CH1 domain including one or more cysteines from the antibody hinge region. F(ab′)2 antibody fragments originally were produced as pairs of Fab′ fragments which have hinge cysteines between them. Other chemical couplings of antibody fragments are also known. In some embodiments, the antibody is a full length antibody (i.e., contains an Fc region, which can be IgG4 for example).

Humanized.

In some embodiments, the antibodies used herein are humanized. Humanized forms of non-human (e.g., murine) antibodies are chimeric immunoglobulins (including full length immunoglobulins), immunoglobulin chains or fragments thereof (such as Fv, Fab, Fab′, F(ab′)2, scFv or other antigen-binding subsequences of antibodies) which contain minimal sequence derived from the non-human immunoglobulin. Humanized antibodies typically include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues that are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin (Jones et al., Nature, 321:522-525 (1986); Riechmann et al., Nature, 332:323-327 (1988); and Presta, Curr. Op. Struct. Biol., 2:593-596 (1992)).

A composite antibody is an antibody that contains sequence segments from different antibodies. Humanized antibodies can be formed of a composite of overlapping human sequences, with one segment of the CDR found in one human sequence and another segment of the same CDR found in another human sequence, each of the two sequences having common sequences at an overlapping region where the segments meet. In an embodiment, the composite human sequence is free of known T cell epitopes. In some embodiments, the composite human sequence does not elicit an immune response in humans. In any of the embodiments, the subject can be human and the antibody can be a humanized antibody. In any of the embodiments, the antibody can be a composite antibody. In any of the embodiments, the subject can be human and the antibody can be a fully human antibody. A fully human antibody is an antibody consisting only of human amino acid sequences.

Further details respecting antibodies and general methods of making antibodies can be found in U.S. patent application publication number 2013/0136735, the entire disclosure of which is incorporated herein by reference.

The antibodies selectively bind their targets, such as CD33 on blast cells. An antibody that selectively binds its target cell(s) means it has the ability to be used in vitro or in vivo to bind to and distinguish such target bearing tissue from other tissue types of the species, including other closely related cell types under the conditions in which the antibody is used, such as under physiologic conditions. In some embodiments, the antibody selectively binds human blast cells that express CD33. In some embodiments, the antibody selectively binds to any region of CD33. In some embodiments, the antibody selectively binds to the IgV domain of CD33. In some embodiments, the antibody is GO. In some embodiments, the antibody is SGN-CD33A. In some embodiments, the antibody is hP67.7. In some embodiments, the antibody is hP67.7 linked to a toxin. The antibody can be any antibody or antigen binding fragment thereof that selectively binds CD33 and is linked to a toxin.

Aspects of the invention relate to treatment with an antibody drug conjugate (ADC), such as an antibody or antigen binding fragment thereof that selectively binds to CD33, which is directly linked to a toxin or linked to a toxin through a linker.

ADCs and Toxins.

Antibodies or antigen binding fragments thereof of the disclosure may be conjugated (covalently or non-covalently linked) to a toxin or they may be linked to a toxin through a linker. The toxin may be any toxin that can elicit a therapeutic effect. The toxin may be an enzymatically active toxin of bacterial, fungal, plant or animal origin or a synthetic toxin, or fragments thereof.

The use of antibody-drug conjugates (ADCs), e.g., immunoconjugates, for the local delivery of cytotoxic or cytostatic agents to kill or inhibit tumor cells in the treatment of cancer (Syrigos and Epenetos (1999) Anticancer Research 19:605-614; Niculescu-Duvaz and Springer (1997) Adv. Drg Del. Rev. 26:151-172; U.S. Pat. No. 4,975,278) theoretically allows targeted delivery of the drug moiety to tumors, and intracellular accumulation therein, where systemic administration of these unconjugated drug agents may result in unacceptable levels of toxicity to normal cells as well as the tumor cells sought to be eliminated (Baldwin et al., Lancet pp., 1986: 603-05; Thorpe, (1985) “Antibody Carriers Of Cytotoxic Agents In Cancer Therapy: A Review,” in Monoclonal Antibodies '84: Biological And Clinical Applications, A. Pinchera et al. (ed.s), pp. 475-506). Efforts to design and refine ADC have focused on the selectivity of monoclonal antibodies (mAbs) as well as drug-linking and drug-releasing properties. Both polyclonal antibodies and monoclonal antibodies have been reported as useful in these strategies (Rowland et al., (1986) Cancer Immunol. Immunother., 21:183-87). Drugs used in these methods include daunomycin, doxorubicin, methotrexate, and vindesine (Rowland et al., (1986) supra).

Toxins useful as therapeutics are known to those skilled in the art. Toxins used in antibody-toxin conjugates include bacterial toxins such as diphtheria toxin, plant toxins such as ricin, small molecule toxins such as geldanamycin (Mandler et al (2000) Jour. of the Nat. Cancer Inst. 92(19):1573-1581; Mandler et al (2000) Bioorganic & Med. Chem. Letters 10: 1025-1028; Mandler et al (2002) Bioconjugate Chem. 13:786-791), maytansinoids (US 20050169933 A1; EP 1391213; Liu et al., (1996) Proc. Natl. Acad. Sci. USA 93:8618-8623), and calicheamicin (Lode et al (1998) Cancer Res. 58:2928; Hinman et al (1993) Cancer Res. 53:3336-3342). Other toxins include plant and bacterial toxins, such as, abrin, alpha toxin, exotoxin, gelonin, pokeweed antiviral protein, and saporin. Toxins can effect their cytotoxic and cytostatic effects by mechanisms including tubulin binding, DNA binding, or topoisomerase inhibition.

In some embodiments, a toxin is linked to an antibody or an antigen binding fragment thereof, through a linker.

Linkers.

Linkers of the disclosure can be any chemical linker. The linker can be a peptide linker. In some embodiments, the peptide linker ranges from about 2 to about 25 amino acids in length. In some embodiments, the peptide linker is 20 amino acids in length. In some embodiments, the peptide linker ranges from about 4 to about 16 amino acids in length. In some embodiments, the peptide linker is 2, 3, 4, 5, 6, 7, 8, 9, 10, 11, 12, 13, 14, 15, 16, 17, 18, 19, 20, 21, 22, 23, 24, or 25 amino acids in length. In some embodiments, the peptide linker is longer than 25 amino acids in length. Linkers of the disclosure can be any cleavable or non-cleavable linker. In some embodiments, a peptide linker provides a protease-dependent cleavable site. Examples of protease-cleavable peptide linkers include, without limitation, the MMP sensitive linker and the factor Xa-sensitive linker IEGR. The art is familiar with a variety of cleavable sequences that may be employed for the methods provided herein, for example those disclosed in Chen et al., Adv. Drug Deliv. Rev. (2013), 65(10): 1357-69).

In some embodiments of the present invention, a flexible peptide linker can be used. A flexible peptide linker is preferably about 25 or fewer amino acids in length. In some embodiments, a flexible peptide linker is 20 amino acids in length. In some embodiments, a peptide linker contains about 20 or fewer amino acid residues, e.g., 2, 3, 4, 5, 6, 7, 8, 9, 10, 11, 12, 13, 14, 15, 16, 17, 18, 19, and 20. In some embodiments, a peptide linker contains about 12 or fewer amino acid residues, e.g., 3, 4, 5, 6, 7, 8, 9, 10, 11, and 12. In some cases, a peptide linker comprises two or more of the following amino acids: glycine, serine, alanine, and threonine. In some embodiments, the flexible peptide linker is a glycine-serine linker.

In some embodiments, the glycine-serine linker is represented by the formula (GS)ₙ, wherein n is 1, 2, 3, 4, 5, 6, 7, 8, 9, 10, 11, or 12. In some embodiments, the glycine-serine linker is represented by the formula (GGGGS)ₙ, wherein n is 1, 2, 3, 4, or 5.

Alternatively, a linking molecule may be a non-peptide linker. As used herein, a “non-peptide linker” refers to a biocompatible polymer including two or more repeating units linked to each other. Examples of the non-peptide polymer include but are not limited to: polyethylene glycol (PEG), polypropylene glycol (PPG), co-poly (ethylene/propylene) glycol, polyoxyethylene (POE), polyurethane, polyphosphazene, polysaccharides, dextran, polyvinyl alcohol, polyvinylpyrrolidones, polyvinyl ethyl ether, polyacryl amide, polyacrylate, polycyanoacrylates, lipid polymers, chitins, hyaluronic acid, and heparin. For more detailed descriptions of non-peptide linkers useful for Fc fusion molecules, see, for example, WO/2006/107124, which is incorporated by reference herein. Typically such linkers will have a range of molecular weight of from about 1 kDa to 50 kDa. For example, a typical PEG has a molecular weight of about 1 to 5 kDa, and polyethylene glycol has a molecular weight of about 5 kDa to 50 kDa, and more preferably about 10 kDa to 40 kDa.

The present disclosure shows that a CD33 protein isoform produced from an alternatively spliced transcript of CD33 lacking exon 2 lacks the IgV domain. Absence of the IgV domain of CD33, which can be indicated by the absence of exon 2 in CD33 mRNA or of the absence of amino acids encoded by exon 2 in the CD33 protein prevents binding of GO or other immunoconjugates targeting this region of CD33.

Aspects of the invention relate to determining whether a subject is likely to benefit from treatment with an agent that selectively binds to CD33. In some embodiments, an assay is performed on a biological sample from a subject with a cancer expressing CD33 to determine whether the subject is likely to benefit from treatments disclosed herein. One of ordinary skill in the art would appreciate that a variety of biological samples could be compatible with aspects of the invention. In some embodiments, assays are performed on tissue from a subject or a derivative of the tissue. In some embodiments, the subject has been diagnosed with a cancer expressing CD33. In some embodiments, an assay is performed to detect the presence of one or more CD33 SNPs. Any suitable assay for detection of a SNP may be compatible with aspects of the invention.

Single Nucleotide Polymorphism (SNP).

Single nucleotide polymorphisms (SNPs) are single-nucleotide substitutions of one base for another that occur in more than one percent of the general population. SNPs occur throughout DNA in the human genome, at about one in every 300 nucleotides. To be classified as a SNP, two or more versions of a sequence must each be present in at least one percent of the general population.

In the present disclosure, six common SNPs: rs12459419-Ala14Val (rs12459419); rs2455069-Arg60Gly (rs2455069); rs35112940-Arg304Gly (rs35112940); rs61736475-Ser305Pro (rs61736475); promoterSNP-rs3865444 (rs3865444); and 3′UTRSNP-rs1803254 (rs1803254) in CD33 were genotyped and screened for association with clinical outcome, mRNA levels, and cell surface CD33 expression levels in 942 de novo AML patients enrolled in a COGAAML0531 trial. Nucleic acid sequences flanking each of the SNPs are shown in Table 1.

SNPs can be identified using various assays including, e.g., without limitation, DNA sequencing, RNA sequencing, primer extension, enzyme-based methods, restriction fragment length polymorphism, PCR-based methods, PCR-RFLP, allele-specific PCR, flap endonuclease, 5′-nuclease, oligonucleotide ligation assay, other post-amplification methods based on physical properties of DNA, single strand conformation polymorphism, temperature gradient gel electrophoresis, denaturing high performance liquid chromatography, high-resolution melting of the entire amplicon, use of DNA mismatch-binding proteins, SNPlex, surveyor nuclease assay, and hybridization-based methods including dynamic allele-specific hybridization, molecular beacons, and SNP microarrays. Other assays known to one of ordinary skill in the art may also be used to identify or genotype SNPs.

As described in the Examples, the presence of a variant single-nucleotide polymorphism (SNP) allele for the SNP rs12459419 (genotype TC or TT) in CD33 increases production of alternatively spliced transcript lacking exon 2 (p=1.41e-10).

In some embodiments, a subject is considered likely to benefit from treatment with an agent that selectively binds to CD33, such as GO if the subject exhibits a genotype of CC for the CD33 SNP rs12459419. Accordingly, in some embodiments, an agent that selectively binds to CD33, such as GO is administered to a subject who exhibits a genotype of CC for the CD33 SNP rs12459419. In some embodiments, a subject is not considered likely to benefit from treatment with an agent that selectively binds to CD33, such as GO if the subject exhibits a genotype of TC or TT genotype for the CD33 SNP rs12459419. Accordingly, in some embodiments, an agent that selectively binds to CD33, such as GO is not administered to a subject who exhibits a genotype of TC or TT for the CD33 SNP rs12459419.

In some embodiments, a subject is considered likely to benefit from treatment with an agent that selectively binds to CD33, such as GO if the subject exhibits a genotype of CC for the CD33 SNP rs3865444. Accordingly, in some embodiments, an agent that selectively binds to CD33, such as GO is administered to a subject who exhibits a genotype of CC for the CD33 SNP rs3865444. In some embodiments, a subject is not considered likely to benefit from treatment with an agent that selectively binds to CD33, such as GO if the subject exhibits a genotype of CA or AA for the CD33 SNP rs3865444. Accordingly, in some embodiments, an agent that selectively binds to CD33, such as GO is not administered to a subject who exhibits a genotype of CA or AA for the CD33 SNP rs3865444.

In some embodiments, an immunoassay is used to detect presence of a CD33 antigen. In some embodiments, an immunoassay can be used to detect presence of amino acids encoded by exon 2 of CD33. In some embodiments, binding of GO to the CD33 protein indicates presence of the CC genotype for rs12459419. In some embodiments, lack of binding of GO to the CD33 protein indicates presence of the TC or TT genotype for rs12459419. In some embodiments, detection of amino acids encoded by exon 2 of CD33 can indicate whether the genotype for SNP rs12459419 is wild-type (genotype CC) or variant (genotype TC or TT).

The antibodies or antigen binding fragments of the disclosure are, for example, suited for use in immunoassays in which they can be utilized in liquid phase or bound to a solid phase carrier. Examples of immunoassays which can utilize the antibody or antigen binding fragments are competitive and non-competitive immunoassays in either a direct or indirect format. Examples of such immunoassays are the Enzyme Linked Immunoassay (ELISA), radioimmunoassay (RIA), the sandwich (immunometric assay), flow cytometry, the western blot assay, immunoprecipitation assays, immunohistochemistry, immuno-microscopy, lateral flow immuno-chromatographic assays, and proteomics arrays. The antigens and antibodies or antigen binding fragments can be bound to many different solid supports (e.g., carriers, membrane, columns, proteomics array, etc.).

In some embodiments, an assay used to detect the presence or absence of amino acids encoded by exon 2 of CD33 can be a protein sequencing assay, e.g., mass spectrometry or Edman degradation.

TABLE 1
CD33 SNPs identified by sequencing and genotyped in the present study

| CD33 SNP | rs# | Location | Flanking sequence | SEQ ID NO: |
|---|---|---|---|---|
| Ala14Val | rs12459419 | Exon 2 | CCCACAGGGG-C-CCTGGCTATG | 1 |
| promoterSNP | rs3865444 | Promoter | CTAAACACCC-C-ATGGATCTAG | 2 |
| 3′UTR | rs1803254 | 3′UTR | CATTTGCTAA-G-TGTATGATGT | 3 |
| Arg304Gly | rs35112940 | Exon 6 | CCCTACCACA-G-GGTCAGCCTC | 4 |
| Arg69Gly | rs2455069 | Exon 2 | CCATTATATCC-A-GGGACTCTCC | 5 |
| Ser305Pro | rs61736475 | Exon 6 | TACCACAGGG-T-CAGCCTCCCC | 6 |

Hybridization.

The nucleic acid sequence of the flanking sequences of the SNPs of the disclosure are provided in SEQ ID NOs: 1 to 6, in Table 1. The amino acid sequence of the variant SNPs encoded by variant SNPs of SEQ ID NO: 1 (variant SNP genotypes TC and TT of rs12459419) excludes amino acids encoded by exon 2 of CD33. The details of each SNP and its variant genotypes are useful for designing nucleic acid probes or primers which may be used to detect the mutant forms of the genes in individuals. For example, a nucleic acid probe may be designed which binds specifically to a sequence of CD33 which includes one or more of the variant SNPs (i.e. it either binds specifically to the variant SNP and not to the wild-type SNP, or it binds preferentially to the variant SNP as opposed to the wild-type). Typically, the probe will have a nucleic acid sequence that is complementary to a sequence of CD33 which includes one or more of the SNPs identified in Table 1. Suitably, the probe comprises a nucleic acid sequence which hybridizes under conditions of suitable stringency to at least 7, at least 14, at least 25, 50, 75, 100, 150, 200, 250, 300, 350, or 400 consecutive nucleotides of the sequence of the CD33 gene (SEQ ID NO: 7) which includes at least one of the SNPs rs12459419, rs3865444, rs1803254, rs35112940, and rs2455069.

Thus, in a further embodiment, there is provided an oligonucleotide which is complementary to a sequence of the CD33 gene which includes at least one of the SNPs indicated in Table 1. Typically, the oligonucleotide is a probe or a primer. Ideally, the primer is a primer for PCR nucleic acid amplification, ideally RT-PCR amplification. In one embodiment, the oligonucleotide consists of at least 7, at least 14, at least 25, 50, 75, 100, 150, 200, 250, 300, 350, or 400 consecutive nucleotides. A sample of myeloid lineage or blast cells from a subject with a cancer expressing CD33 may be isolated, the DNA extracted, and then assayed using a probe of the disclosure for the presence of one of the variant SNPs indicated in Table 1. Likewise, an oligonucleotide primer of the disclosure may be used to perform RT-PCR on the DNA sample. As the primer is designed to bind with the target DNA only when a desired variant SNP or desired wild-type SNP is present, amplification will only take place when the variant or wild-type SNP, respectively is present. The design of primers and probes of the disclosure, and their use in determining the presence of any wild-type or variant SNPs of the disclosure will be well known to a person skilled in the art.

CD33SNP_Score.

In some embodiments, a genotype score is determined for one or more of the SNPs of the disclosure. The genotype score can be used to determine whether a subject is likely to respond to treatment with an agent that selectively binds to CD33. The genotype scores can be −2, −1, 0, 1, or 2. For each of the SNPs rs12459419/rs3865444, rs1803254 and rs35112940, patient genotypes are given a genotype score of 0 (wt/wt), −1 (wt/var) or −2 (var/var) based on the number of low expression allele. Since rs2455069 was associated with high expression genotype, genotype scores were 0 (wt/wt), 1 (wt/var) and 2 (var/var). A CD33SNP_Score can be calculated by adding the directional genotype scores of the SNPs: rs12459419 or rs3865444, rs1803254, rs35112940, and rs2455069. Therefore the CD33SNP_Score=Σ (genotype scores of rs12459419 (or rs3865444), rs1803254, rs2455069 and rs35112940).

In some embodiments, a CD33SNP_Score greater than or equal to zero indicates that the subject is likely to benefit from treatment. In some embodiments, a genotype score equal to zero for SNPs rs12459419 and/or rs3865444 indicates that the subject is likely to benefit from treatment. In some embodiments, a CD33SNP_Score greater than or equal to zero indicates that the subject is likely to benefit from treatment. In some embodiments, a genotype score of less than zero for SNPs rs12459419 and/or rs3865444 indicates that the subject is not likely to benefit from treatment. In some embodiments, the subject is likely to benefit from treatment with an agent that selectively binds amino acids encoded by exon 2 of CD33 if the CD33SNP_Score for the subject is greater than or equal to zero. In some embodiments, the subject is not likely to benefit from treatment with an agent that selectively binds amino acids encoded by exon 2 of CD33 if the CD33SNP_Score for the subject is less than zero. In some embodiments, the subject is likely to benefit from treatment with an agent that selectively binds amino acids encoded by exon 2 of CD33 if the genotype score for SNPs rs12459419 and/or rs3865444 is equal to zero for the subject. In some embodiments, the subject is not likely to benefit from treatment with an agent that selectively binds amino acids encoded by exon 2 of CD33 if the genotype score for SNPs rs12459419 and/or rs3865444 is less than zero for the subject.

Subject.

“Subject” means a mammal, such as a human, a nonhuman primate, a dog, a cat, a sheep, a horse, a cow, a pig or a goat. In an important embodiment, the mammal is a human. The subject as used herein can be an adult subject or a pediatric subject.

Biological Sample.

A “biological sample” from a subject can include any cellular, tissue, bone marrow, or blood sample from the subject. Any type of biological sample appropriate for conducting assays described herein can be compatible with aspects of the invention, as would be understood by one of ordinary skill in the art.

Treatment.

“Treat”, “treating” and “treatment” encompass an action that occurs while a subject is suffering from a condition which reduces the severity of the condition (or a symptom associated with the condition) or retards or slows the progression of the condition (or a symptom associated with the condition). This is therapeutic treatment.

Subjects are treated with effective amounts of the agents of the disclosure. An “effective amount” of an agent generally refers to an amount sufficient to elicit the desired biological response, i.e., treat the condition. As will be appreciated by those of ordinary skill in this art, the effective amount of an agent described herein may vary depending on such factors as the condition being treated, the mode of administration, the therapy, if any, with which it is combined, and the age and health of the subject.

For therapeutic treatment, a therapeutically effective amount is an amount sufficient to provide a therapeutic benefit in the treatment of a condition or to reduce or eliminate one or more symptoms associated with the condition. This may encompass an amount that improves overall therapy, reduces or avoids symptoms or causes of the condition, or enhances the therapeutic efficacy of another therapeutic agent.

In some embodiments, the subject is treated with chemotherapy and/or radiation in addition to treatment with an agent that selectively binds to CD33, such as GO. In some embodiments, the subject is treated with chemotherapy within 30 days of administration of the agent that selectively binds CD33. In some embodiments, the subject is treated with radiation therapy within 30 days of administration of the agent that selectively binds CD33.

In general, effective amounts are administered to reduce the number of or kill cancer cells. In connection with a specific disease or condition, a therapeutically effective amount can halt the development of, inhibit the progression of, reverse the development of, or otherwise reduce or ameliorate one or more symptoms of the disease or condition, for example, one or more symptoms of cancer.

Pharmaceutical Compositions.

Agents that selectively bind to CD33, such as humanized antibodies, biologics and other molecules can be administered for the treatment of various disorders in the form of pharmaceutical compositions. Such compositions include the therapeutic(s) and one or more other pharmaceutically acceptable components. See Remington's Pharmaceutical Science (15th ed., Mack Publishing Company, Easton, Pa. (1980)). The preferred form depends on the intended mode of administration and therapeutic application. The compositions can also include, depending on the formulation desired, pharmaceutically-acceptable, non-toxic carriers or diluents, which are defined as vehicles commonly used to formulate pharmaceutical compositions for animal or human administration. The diluent is selected so as not to adversely affect the biological activity of the antibody. Examples of such diluents are distilled water, physiological phosphate-buffered saline, Ringer's solutions, dextrose solution, and Hank's solution. In addition, the pharmaceutical composition or formulation may also include other carriers or nontoxic, nontherapeutic, nonimmunogenic stabilizers and the like.

Pharmaceutical compositions can also include large, slowly metabolized macromolecules such as proteins, polysaccharides such as chitosan, polylactic acids, polyglycolic acids and copolymers (such as latex functionalized SEPHAROSE™ (GE Healthcare Bio-Sciences Ltd.), agarose, cellulose, and the like), polymeric amino acids, amino acid copolymers, and lipid aggregates (such as oil droplets or liposomes).

Pharmaceutical compositions may be injectable compositions. Injectable compositions include solutions, suspensions, dispersions, and the like. Injectable solutions, suspensions, dispersions, and the like may be formulated according to techniques well-known in the art (see, for example, Remington's Pharmaceutical Sciences, Chapter 43, 14th Ed., Mack Publishing Co., Easton, Pa.), using suitable dispersing or wetting and suspending agents, such as sterile oils, including synthetic mono- or diglycerides, and fatty acids, including oleic acid.

Injectable compositions that include an agent useful in the invention may be prepared in water, saline, isotonic saline, phosphate-buffered saline, citrate-buffered saline, and the like and may optionally mixed with a nontoxic surfactant. Dispersions may also be prepared in glycerol, liquid polyethylene, glycols, DNA, vegetable oils, triacetin, and the like and mixtures thereof. Under ordinary conditions of storage and use, these preparations may contain a preservative to prevent the growth of microorganisms. Pharmaceutical dosage forms suitable for injection or infusion include sterile, aqueous solutions or dispersions or sterile powders comprising an active ingredient which powders are adapted for the extemporaneous preparation of sterile injectable or infusible solutions or dispersions. Preferably, the ultimate dosage form is a sterile fluid and stable under the conditions of manufacture and storage. A liquid carrier or vehicle of the solution, suspension or dispersion may be a solvent or liquid dispersion medium comprising, for example, water, ethanol, a polyol such as glycerol, propylene glycol, or liquid polyethylene glycols and the like, vegetable oils, nontoxic glyceryl esters, and suitable mixtures thereof. Proper fluidity of solutions, suspensions or dispersions may be maintained, for example, by the formation of liposomes, by the maintenance of the desired particle size, in the case of dispersion, or by the use of nontoxic surfactants. The prevention of the action of microorganisms can be accomplished by various antibacterial and antifungal agents, for example, parabens, chlorobutanol, phenol, sorbic acid, thimerosal, and the like. Isotonic agents such as sugars, buffers, or sodium chloride may be included. Prolonged absorption of the injectable compositions can be brought about by the inclusion in the composition of agents delaying absorption—for example, aluminum monostearate hydrogels and gelatin. Solubility enhancers may be added.

Sterile injectable compositions may be prepared by incorporating the therapeutic in the desired amount in the appropriate solvent with various other ingredients, e.g. as enumerated above, and followed by sterilization, as desired, by, for example filter sterilization. In the case of sterile powders for the preparation of sterile injectable solutions, methods of preparation include vacuum drying and freeze-drying techniques, which yield a powder of the active ingredient plus any additional desired ingredient present in a previously sterile-filtered solution. Any suitable sterilization process may be employed, such as filter sterilization, e.g. 0.22 micron filter or nanofiltration, gamma or electron beam sterilization, or pulsed white light. Other suitable sterilization processes include UtiSter (Pegasus Biologics, Irvine Calif.) and those described in, e.g., U.S. Pat. Nos. 6,946,098 and 5,730,933.

In various embodiments, the final solution typically is adjusted to have a pH between about 4 and about 9, between about 5 and about 7, between about 5.5 and about 6.5, or about 6. The pH of the composition may be adjusted with a pharmacologically acceptable acid, base or buffer. Hydrochloric acid is an example of a suitable acid, and sodium hydroxide is an example of a suitable base. The hydrochloric acid or sodium hydroxide may be in any suitable form, such as a 1N solution

A resultant injectable solution preferably contains an amount of one or more therapeutics effective to treat a disease. In various embodiments, a therapeutic such as an antibody is present in an injectable composition at a concentration between about 0.0001 mg/ml and about 50 mg/ml. In various embodiments, an antibody is present in an injectable composition at a concentration between about 0.01 mg/mL and about 10 mg/mL.

Agents, such as antibodies, also may be administered via other modes of administration known in the art. Such modes of administration include inhalation, ingestion and topical application. Oral administration is also possible for therapeutics, although this form of administration is more challenging for certain biologics such as antibodies.

### Kits

In certain aspects, the disclosure provides kits. Kits can include an agent that selectively binds to CD33. In some embodiments, the agent is conjugated to a toxin. In some embodiments, the agent is linked to a toxin through a linker. In some embodiments, the agent is in sterile container(s). In some embodiments, the kit comprises instructions for administration of the kit components. In some embodiments, the instructions indicate the use of the agent to treat a subject if the genotype of the subject for the CD33 SNP rs12459419 is CC and/or if the genotype of the subject for the CD33 SNP rs3865444 is CC. In some embodiments, the kit includes a pharmaceutical preparation vial, a pharmaceutical preparation diluent vial, and the agent. The diluent vial contains a diluent such as physiological saline for diluting what could be a concentrated solution or lyophilized powder of a composition of the disclosure. In some embodiments, the instructions include instructions for mixing a particular amount of the diluent with a particular amount of a concentrated pharmaceutical composition, whereby a final formulation for injection or infusion is prepared. In some embodiments, the instructions include instructions for use in a syringe or other administration device. In some embodiments, the instructions include instructions for treating a patient with an effective amount of a composition of the disclosure. It also will be understood that the containers containing the preparations, whether the container is a bottle, a vial with a septum, an ampoule with a septum, an infusion bag, and the like, may contain indicia such as conventional markings which change color when the preparation has been autoclaved or otherwise sterilized.

### EQUIVALENTS

Those skilled in the art will recognize, or be able to ascertain using no more than routine experimentation, many equivalents to the specific embodiments of the invention described herein. Such equivalents are intended to be encompassed by the following claims.

Unless otherwise defined herein, scientific and technical terms used in connection with the present disclosure shall have the meanings that are commonly understood by those of ordinary skill in the art. Further, unless required by context, singular terms shall include pluralities and plural terms shall include the singular. The methods and techniques of the present disclosure are generally performed according to conventional methods well-known in the art. Generally, nomenclatures used in connection with, and techniques of biochemistry, enzymology, molecular and cellular biology, microbiology, genetics and protein and nucleic acid chemistry and hybridization described herein are those well-known and commonly used in the art. The methods and techniques of the present disclosure are generally performed according to conventional methods known in the art and as described in various general and more specific references that are cited and discussed throughout the present specification unless otherwise indicated.

Exemplary embodiments of the disclosure will be described in more detail by the following examples. These embodiments are exemplary of the disclosure, which one skilled in art will recognize is in no way limited to the exemplary embodiments. The entire contents of all of the references (including literature references, issued patents, published patent applications, and co-pending patent applications) cited throughout this application are hereby expressly incorporated by reference, in particular for the teaching that is referenced hereinabove. However, the citation of any reference is not intended to be an admission that the reference is prior art.

### EXAMPLES

### Example 1: CD33 Genetic Variation Predicts Clinical Response to Gemtuzumab Ozogamicin Vs. Standard Therapy in De Novo AML

Gemtuzumab Ozogamicin (GO) is a CD33-targeting agent that holds promise in both adult and pediatric AML populations. Two Meta-analysis reports including 11 and 5 randomized control trials suggest GO provided significant survival benefit in patients with favorable cytogenetics (9,10). Within pediatric AML, two trials from Children's Oncology Group COG-AAML03P1, (all patients received GO based chemotherapy) and more recently AAML0531 (patients were randomized to receive either standard five-course chemotherapy with or without addition of 2 doses of GO) demonstrated that addition of GO improved event free survival (EFS) and overall survival (OS) in AAML03P1 (11) and EFS and reduction in relapse risk (RR) within AAML0531(12). These results indicate that GO has a potential to improve outcome in AML and was prematurely with drawn from market (13,14).

In AML, wide inter-patient variation in leukemic CD33 cell surface intensity as well as association with negative prognostic factors such as FLT3-ITD has been reported (15). Recent investigation from AAML0531 shows benefit from GO addition in patients with high leukemic cell surface CD33 levels and which was not seen in patient with low levels CD33 (16). We have previously shown association of CD33 SNPs with leukemic cell surface CD33 intensity and clinical outcome in patients within the AAML03P1 study (17). As the AAML03P1 clinical trial did not have a randomized control arm, it was not possible to investigate whether CD33 SNPs demonstrated differences in clinical outcome in patients in GO vs. No-GO arm. Here, we report results of clinical significance of 6 common CD33 SNPs by GO vs. No-GO treatment arms in a large cohort (n=1022) of de novo AML patients treated under COG AAML0531 trial. These results demonstrate significant clinical impact of a splicing and coding SNP rs12459419 on clinical outcome and provide rationale for selection of patients based on the genotype for GO based chemotherapy. Presence of the SNP results in exon 2 skipping and thus deletion of the epitope targeted by GO, thereby compromising response to GO.

### Methods

### Patients and Treatment

Pediatric patients enrolled in the COG trial AAML0531 were included for this study. Details of the study design, treatment regimen and clinical outcome have been described in detail previously (Gamis et al., Journal of clinical oncology: official journal of the American Society of Clinical Oncology, 2014; 32(27):3021-32). Overall, 1022 patients between ages 0-29 years with de novo AML were randomly assigned to either standard five course chemotherapy (No-GO, n=511) or to the same chemotherapy with addition of two doses of 3 mg/m2 of GO (GO, n=511) during induction course I and intensification II. Low risk (LR) features were presence of t(8; 21), inv(16) or t(16; 16); high risk (HR) features included presence of monosomy 7, monosomy 5/5q deletion, or persistent disease at end of induction 1. All high-risk group patients received allogeneic stem cell transplant. Patients not assigned to low or high-risk groups were classified as intermediate risk (IR) patients and these patients received SCT if available. For this study, in addition to features listed above, presence of FLT-3/ITD mutations was considered high-risk and presence of NPM1 or CEBP mutation with absence of FLT3/ITD was considered low-risk. All AAML0531 patient specimens with consent for biology studies were used in this study. The institutional review boards of all participating institutions approved the clinical protocol and the COG Myeloid Disease Biology Committee approved this research.

### Genotyping of CD33 SNPs

Six SNPs in CD33, including: a promoter SNP—rs3865444; 4 coding SNPs: rs12459419-Ala14Val, rs2455069-Arg60Gly, rs35112940-Arg304Gly and rs61736475-Ser305Pro; and a 3′UTR SNP—rs1803254 were genotyped using Sequenome platform at Biomedical Genomics Center at University of Minnesota (Minneapolis, Minn.). All the 6 SNPs had a call rate of >0.98.

### CD33 Expression Levels on Leukemic Cells

CD33 expression levels as determined by mean fluorescent intensity (MFI) of myeloid progenitor cells, as defined by CD45 low and side scatter, was determined by flow cytometry using the P67.7 antibody that specifically recognizes the IgV domain of CD33 as described previously (Pollard et al., Journal of clinical oncology: official journal of the American Society of Clinical Oncology, 2016; 34(7):747-55; Mortland et al., Clinical cancer research: an official journal of the American Association for Cancer Research, 2013; 19(6):1620-7; Pollard et al., Blood, 2012; 119(16):3705-11; and Wells, Leukemia research, 2008; 32(6).).

mRNA Levels of CD33 and its Isoforms

CD33-WT and alternatively spliced transcript levels, as well as exon levels were obtained from RNA-seq data from 80 patients. Additionally CD33-WT and D2 splice variant lacking exon 2 was quantitated in 30 samples (10 with each genotype) with real time PCR using isoform specific primers.

RNA Seq:

RNA-seq data was available from 80 patients. CD33 mRNA transcript levels as well as levels of each exon was obtained. Additionally, the expression of a CD33-D2 isoform with deletion of exon 2 expressed as the “percent-spliced-in” (PSI, or ψ) value was obtained. PSI represents percentage of all mRNAs that correspond to the isoform with exclusion of exon 2.

Real-Time Quantitative PCR Analysis:

mRNA expression levels of CD33 WT and D2-splice variant isoforms were quantitated in 30 patients selected based on genotype for rs12459419 (10 in each genotype) from the COG-AML0531 study. Briefly, total RNA was extracted and cDNA was synthesized using High Capacity cDNA reverse transcription kit (Life Technologies, USA). Quantitative real-time PCR was carried out using ABI7900-HT Real-time PCR system and QuantiTect SYBR Green PCR Kit. To quantify expression of total CD33: Forward, 5-TGTTCCACAGAACC CAACAA-3 and reverse, 5-GGCTGTAACACCAGCTC CTC-3 primers corresponding to sequences within exons 4 and 5, respectively were used. To quantify expression of D2-CD33: Forward, 5-CCCTGCTGTGGGCAGACTTG-3; and reverse, 5-GCACCGAGGAGTGAGTAGTCC-3 primers corresponding to sequences at the exon 1-3 junction and exon 3, respectively were used. The relative levels of CD33 isoforms were determined by 2\hat{ }-ΔΔCT method after normalization to GAPDH levels.

CD33 SNPs are not Associated with Patient Characteristics

6 SNPs in the CD33 gene in 942 patients treated in the COG AAML0531 clinical trial were genotyped. Table 8 provides CD33 SNP status with respect to patient and disease characteristics. CD33 SNPs differed in frequency by race; however, rs12459419 (LD SNP rs3865444), rs1803254, rs2455069 and rs35112940 did not show any significant difference in representation by treatment arm, cytogenetics, risk group and FLT3-ITD status, or whether patients received SCT in the study or not. rs61736475, a less commonly occurring SNP, differed significantly among cytogenetic features, risk group, FLI3-ITD status and whether or not patients received SCT.

TABLE 8
Patient and disease characteristics by CD33 SNPs.

| | | rs12459419 | | | | | | |
|---|---|---|---|---|---|---|---|---|
| | | Genotype Frequency | | | | | | |
| | Popu- | | | | | | | Geno- |
| | lation | CC | | CT | | TT | | types |
| Characteristics | N | N | % | N | % | N | % | p |
| Gender | | | | | | | | |
| Male | 414 | 209 | 50% | 163 | 52% | 41 | 48% | 0.852 |
| Female | 403 | 206 | 50% | 153 | 48% | 44 | 52% | |
| Race | | | | | | | | |
| White | 599 | 283 | 74% | 247 | 86% | 69 | 99% | <0.001 |
| Black | 93 | 68 | 18% | 24 | 8% | 1 | 1% | <0.001 |
| Other | 44 | 29 | 8% | 15 | 5% | 0 | 0% | 0.037 |
| Unknown | 81 | 35 | | 30 | | 15 | | |
| Treatment Arm | | | | | | | | |
| Arm A | 408 | 204 | 49% | 158 | 50% | 46 | 54% | 0.707 |
| Arm B | 409 | 211 | 51% | 158 | 50% | 39 | 46% | |
| Cytogenetics | | | | | | | | |
| Normal | 180 | 104 | 26% | 58 | 19% | 18 | 22% | 0.083 |
| t(8:21) | 117 | 61 | 15% | 46 | 15% | 10 | 12% | 0.761 |
| inv(16) | 96 | 41 | 10% | 41 | 13% | 13 | 16% | 0.247 |
| Abn 11 | 162 | 80 | 20% | 65 | 21% | 17 | 20% | 0.925 |
| t(6:9) | 11 | 7 | 2% | 2 | 1% | 2 | 2% | 0.328 |
| monosomy 7 | 16 | 6 | 1% | 9 | 3% | 1 | 1% | 0.348 |
| del (7q) | 11 | 6 | 1% | 3 | 1% | 2 | 2% | 0.591 |
| −5/5q | 11 | 8 | 2% | 3 | 1% | 0 | 0% | 0.270 |
| +8 | 51 | 23 | 6% | 26 | 8% | 2 | 2% | 0.099 |
| Other | 139 | 66 | 16% | 55 | 18% | 18 | 22% | 0.507 |
| Unknown | 23 | | | | | | | |
| FLT3/ITD | | | | | | | | |
| No | 671 | 341 | 84% | 261 | 83% | 68 | 82% | 0.917 |
| Yes | 133 | 66 | 16% | 52 | 17% | 15 | 18% | |
| Unknown | 8 | | | | | | | |
| CEBPA status | | | | | | | | |
| CEBPA mutant | 49 | 30 | 7% | 15 | 5% | 4 | 5% | 0.327 |
| CEBPA WT | 758 | 380 | 93% | 298 | 95% | 79 | 95% | |
| Unknown | 7 | | | | | | | |
| NPM status | | | | | | | | |
| NPM mutant | 64 | 36 | 12% | 23 | 7% | 5 | 6% | 0.622 |
| NPM WT | 742 | 374 | 88% | 289 | 93% | 78 | 94% | |
| Risk group | | | | | | | | |
| Standard | 385 | 192 | 47% | 149 | 48% | 44 | 53% | 0.626 |
| Low | 307 | 159 | 39% | 119 | 38% | 28 | 34% | 0.649 |
| High | 111 | 55 | 14% | 45 | 14% | 11 | 13% | 0.938 |
| MRD at End of | | | | | | | | |
| Induction 1 | | | | | | | | |
| No | 472 | 229 | 55% | 189 | 60% | 53 | 62% | 0.620 |
| Yes | 212 | 111 | 27% | 77 | 24% | 24 | 28% | |
| Received SCT on | | | | | | | | |
| study | | | | | | | | |
| Yes | 128 | 57 | 14% | 56 | 18% | 15 | 18% | 0.297 |
| No | 689 | 358 | 86% | 260 | 82% | 70 | 82% | |
| | | rs1803254 | | | | | | |
| | | Genotype Frequency | | | | | | |
| | Popu- | | | | | | | Geno- |
| | lation | CC | | CT | | TT | | types |
| Characteristics | N | N | % | N | % | N | % | p |
| Gender | | | | | | | | |
| Male | 414 | 21 | 53% | 111 | 50% | 281 | 51% | 0.916 |
| Female | 403 | 19 | 48% | 113 | 50% | 271 | 49% | |
| Race | | | | | | | | |
| White | 599 | 28 | 82% | 133 | 69% | 438 | 86% | <0.001 |
| Black | 93 | 6 | 18% | 47 | 24% | 40 | 8% | <0.001 |
| Other | 44 | 0 | 0% | 13 | 7% | 31 | 6% | 0.306 |
| Unknown | 81 | 6 | | 31 | | 43 | | |
| Treatment Arm | | | | | | | | |
| Arm A | 408 | 24 | 60% | 119 | 53% | 265 | 48% | 0.187 |
| Arm B | 409 | 16 | 40% | 105 | 47% | 287 | 52% | |
| Cytogenetics | | | | | | | | |
| Normal | 180 | 6 | 15% | 49 | 23% | 125 | 23% | 0.483 |
| t(8:21) | 117 | 6 | 15% | 38 | 18% | 73 | 14% | 0.375 |
| inv(16) | 96 | 7 | 18% | 24 | 11% | 64 | 12% | 0.519 |
| Abn 11 | 162 | 7 | 18% | 38 | 18% | 117 | 22% | 0.389 |
| t(6:9) | 11 | 0 | 0% | 3 | 1% | 8 | 1% | 0.739 |
| monosomy 7 | 16 | 0 | 0% | 6 | 3% | 10 | 2% | 0.468 |
| del (7q) | 11 | 1 | 3% | 2 | 1% | 8 | 1% | 0.691 |
| −5/5q | 11 | 0 | 0% | 2 | 1% | 9 | 2% | 0.542 |
| +8 | 51 | 1 | 3% | 13 | 6% | 37 | 7% | 0.528 |
| Other | 139 | 12 | 30% | 41 | 19% | 86 | 16% | 0.065 |
| Unknown | 23 | | | | | | | |
| FLT3/ITD | | | | | | | | |
| No | 671 | 37 | 93% | 180 | 81% | 453 | 84% | 0.221 |
| Yes | 133 | 3 | 8% | 41 | 19% | 89 | 16% | |
| Unknown | 8 | | | | | | | |
| CEBPA status | | | | | | | | |
| CEBPA mutant | 49 | 3 | 8% | 10 | 5% | 36 | 7% | 0.501 |
| CEBPA WT | 758 | 37 | 93% | 212 | 95% | 508 | 93% | |
| Unknown | 7 | | | | | | | |
| NPM status | | | | | | | | |
| NPM mutant | 64 | 2 | 5% | 15 | 7% | 47 | 9% | 0.539 |
| NPM WT | 742 | 38 | 95% | 206 | 93% | 497 | 91% | |
| Risk group | | | | | | | | |
| Standard | 385 | 21 | 53% | 104 | 47% | 260 | 48% | 0.817 |
| Low | 307 | 16 | 40% | 81 | 37% | 209 | 39% | 0.852 |
| High | 111 | 3 | 8% | 36 | 16% | 72 | 13% | 0.274 |
| MRD at End of | | | | | | | | |
| Induction 1 | | | | | | | | |
| No | 472 | 25 | 63% | 126 | 56% | 320 | 58% | 0.572 |
| Yes | 212 | 11 | 28% | 65 | 29% | 136 | 25% | |
| Received SCT on | | | | | | | | |
| study | | | | | | | | |
| Yes | 128 | 8 | 20% | 37 | 17% | 83 | 15% | 0.652 |
| No | 689 | 32 | 80% | 187 | 83% | 469 | 85% | |
| | | rs2455069 | | | | | | |
| | | Genotype Frequency | | | | | | |
| | Popu- | | | | | | | Geno- |
| | lation | CC | | CT | | TT | | types |
| Characteristics | N | N | % | N | % | N | % | p |
| Gender | | | | | | | | |
| Male | 414 | 148 | 47% | 194 | 52% | 65 | 54% | 0.309 |
| Female | 403 | 164 | 53% | 176 | 48% | 55 | 46% | |
| Race | | | | | | | | |
| White | 599 | 201 | 75% | 294 | 85% | 95 | 87% | 0.001 |
| Black | 93 | 31 | 12% | 45 | 13% | 14 | 13% | 0.853 |
| Other | 44 | 36 | 13% | 6 | 2% | 0 | 0% | <0.001 |
| Unknown | 81 | 44 | | 25 | | 11 | | |
| Treatment Arm | | | | | | | | |
| Arm A | 408 | 165 | 53% | 177 | 48% | 60 | 50% | 0.422 |
| Arm B | 409 | 147 | 47% | 193 | 52% | 60 | 50% | |
| Cytogenetics | | | | | | | | |
| Normal | 180 | 76 | 25% | 69 | 19% | 34 | 30% | 0.036 |
| t(8:21) | 117 | 41 | 14% | 57 | 16% | 16 | 14% | 0.695 |
| inv(16) | 96 | 43 | 14% | 37 | 10% | 15 | 13% | 0.289 |
| Abn 11 | 162 | 49 | 16% | 88 | 24% | 22 | 19% | 0.031 |
| t(6:9) | 11 | 5 | 2% | 3 | 1% | 2 | 2% | 0.579 |
| monosomy 7 | 16 | 8 | 3% | 8 | 2% | 0 | 0% | 0.226 |
| del (7q) | 11 | 2 | 1% | 7 | 2% | 1 | 1% | 0.315 |
| −5/5q | 11 | 2 | 1% | 9 | 2% | 2 | 2% | 0.466 |
| +8 | 51 | 17 | 65 | 24 | 7% | 8 | 7% | 0.818 |
| Other | 139 | 60 | 20% | 62 | 17% | 15 | 13% | 0.259 |
| Unknown | 23 | | | | | | | |
| FLT3/ITD | | | | | | | | |
| No | 671 | 253 | 82% | 316 | 87% | 89 | 77% | 0.034 |
| Yes | 133 | 55 | 18% | 49 | 13% | 27 | 23% | |
| Unknown | 8 | | | | | | | |
| CEBPA status | | | | | | | | |
| CEBPA mutant | 49 | 14 | 5% | 25 | 7% | 9 | 8% | 0.344 |
| CEBPA WT | 758 | 294 | 95% | 341 | 93% | 109 | 92% | |
| Unknown | 7 | | | | | | | |
| NPM status | | | | | | | | |
| NPM mutant | 64 | 28 | 9% | 21 | 6% | 14 | 12% | 0.067 |
| NPM WT | 742 | 280 | 91% | 344 | 94% | 104 | 88% | |
| Risk group | | | | | | | | |
| Standard | 385 | 146 | 48% | 1848 | 51% | 47 | 40% | 0.128 |
| Low | 307 | 115 | 38% | 135 | 37% | 51 | 43% | 0.472 |
| High | 111 | 45 | 15% | 45 | 12% | 20 | 17% | 0.408 |
| MRD at End of | | | | | | | | |
| Induction 1 | | | | | | | | |
| No | 472 | 170 | 54% | 229 | 62% | 86 | 72% | 0.178 |
| Yes | 212 | 89 | 29% | 86 | 23% | 31 | 26% | |
| Received SCT on | | | | | | | | |
| study | | | | | | | | |
| Yes | 128 | 51 | 16% | 55 | 15% | 19 | 16% | 0.866 |
| No | 689 | 261 | 84% | 315 | 85% | 101 | 84% | |
| | | rs35112940 | | | | | | |
| | | Genotype Frequency | | | | | | |
| | Popu- | | | | | | | Geno- |
| CD33 SNP | lation | CC | | CT | | TT | | types |
| Patients (N = 817) | N | N | % | N | % | N | % | p |
| Gender | | | | | | | | |
| Male | 414 | 9 | 41% | 82 | 47% | 322 | 52% | 0.3448 |
| Female | 403 | 13 | 59% | 92 | 53% | 298 | 48% | |
| Race | | | | | | | | |
| White | 599 | 20 | 95% | 140 | 89% | 439 | 79% | 0.003 |
| Black | 93 | 1 | 5% | 12 | 8% | 80 | 14% | 0.045 |
| Other | 44 | 0 | 0% | 5 | 3% | 39 | 7% | 0.104 |
| Unknown | 81 | 1 | | 17 | | 62 | | |
| Treatment Arm | | | | | | | | |
| Arm A | 408 | 12 | 55% | 91 | 52% | 305 | 49% | 0.701 |
| Arm B | 409 | 10 | 45% | 83 | 48% | 315 | 51% | |
| Cytogenetics | | | | | | | | |
| Normal | 180 | 4 | 19% | 32 | 19% | 144 | 24% | 0.345 |
| t(8:21) | 117 | 2 | 10% | 22 | 13% | 93 | 15% | 0.568 |
| inv(16) | 96 | 5 | 24% | 21 | 12% | 69 | 11% | 0.227 |
| Abn 11 | 162 | 4 | 19% | 42 | 25% | 116 | 19% | 0.296 |
| t(6:9) | 11 | 0 | 0% | 3 | 2% | 8 | 1% | 0.784 |
| monosomy 7 | 16 | 1 | 5% | 4 | 2% | 11 | 2% | 0.604 |
| del (7q) | 11 | 0 | 0% | 3 | 2% | 8 | 1% | 0.784 |
| −5/5q | 11 | 0 | 0% | 2 | 1% | 9 | 1% | 0.818 |
| +8 | 51 | 0 | 0% | 11 | 6% | 40 | 7% | 0.475 |
| Other | 139 | 5 | 24% | 30 | 18% | 104 | 17% | 0.740 |
| Unknown | 23 | | | | | | | |
| FLT3/ITD | | | | | | | | |
| No | 671 | 16 | 80% | 144 | 83% | 510 | 84% | 0.910 |
| Yes | 133 | 4 | 20% | 29 | 17% | 100 | 16% | |
| Unknown | 8 | | | | | | | |
| CEBPA status | | | | | | | | |
| CEBPA mutant | 49 | 0 | 0% | 10 | 6% | 39 | 6% | 0.498 |
| CEBPA WT | 758 | 20 | 100% | 162 | 94% | 575 | 94% | |
| Unknown | 7 | | | | | | | |
| NPM status | | | | | | | | |
| NPM mutant | 64 | 0 | 0% | 13 | 8% | 51 | 8% | 0.391 |
| NPM WT | 742 | 20 | 100% | 159 | 92% | 562 | 92% | |
| Risk group | | | | | | | | |
| Standard | 385 | 12 | 57% | 86 | 50% | 287 | 47% | 0.526 |
| Low | 307 | 7 | 33% | 61 | 36% | 238 | 39% | 0.655 |
| High | 111 | 2 | 10% | 24 | 14% | 85 | 14% | 0.845 |
| MRD at End of | | | | | | | | |
| Induction 1 | | | | | | | | |
| No | 472 | 15 | 68% | 109 | 63% | 347 | 56% | 0.211 |
| Yes | 212 | 4 | 18% | 39 | 22% | 169 | 27% | |
| Received SCT on | | | | | | | | |
| study | | | | | | | | |
| Yes | 128 | 2 | 9% | 25 | 14% | 101 | 16% | 0.570 |
| No | 689 | 20 | 91% | 149 | 86% | 519 | 84% | |
| | | rs61736475 | | | | | | |
| | | Genotype Frequency | | | | | | |
| | Popu- | | | | | | | Geno- |
| CD33 SNP | lation | CC | | CT | | TT | | types |
| Patients (N = 817) | N | N | % | N | % | N | % | p |
| Gender | | | | | | | | |
| Male | 414 | 2 | 33% | 379 | 52% | 32 | 42% | 0.155 |
| Female | 403 | 4 | 67% | 351 | 48% | 45 | 58% | |
| Race | | | | | | | | |
| White | 599 | 0 | 0% | 565 | 86% | 31 | 44% | <0.001 |
| Black | 93 | 6 | 100% | 48 | 7% | 38 | 54% | <0.001 |
| Other | 44 | 0 | 0% | 43 | 7% | 1 | 1% | 0.189 |
| Unknown | 81 | 0 | | 74 | | 7 | | |
| Treatment Arm | | | | | | | | |
| Arm A | 408 | 3 | 50% | 368 | 50% | 36 | 47% | 0.830 |
| Arm B | 409 | 3 | 50% | 362 | 50% | 41 | 53% | |
| Cytogenetics | | | | | | | | |
| Normal | 180 | 1 | 17% | 163 | 23% | 15 | 20% | 0.761 |
| t(8:21) | 117 | 2 | 33% | 91 | 13% | 23 | 30% | <0.001 |
| inv(16) | 96 | 1 | 17% | 88 | 12% | 7 | 9% | 0.677 |
| Abn 11 | 162 | 1 | 17% | 147 | 21% | 14 | 18% | 0.867 |
| t(6:9) | 11 | 0 | 0% | 10 | 1% | 0 | 0% | 0.556 |
| monosomy 7 | 16 | 0 | 0% | 15 | 2% | 1 | 1% | 0.840 |
| del (7q) | 11 | 0 | 0% | 10 | 1% | 1 | 1% | 0.956 |
| −5/5q | 11 | 0 | 0% | 10 | 1% | 1 | 1% | 0.956 |
| +8 | 51 | 0 | 0% | 48 | 7% | 3 | 4% | 0.515 |
| Other | 139 | 1 | 17% | 126 | 18% | 11 | 14% | 0.768 |
| Unknown | 23 | | | | | | | |
| FLT3/ITD | | | | | | | | |
| No | 671 | 6 | 100% | 591 | 82% | 71 | 93% | 0.025 |
| Yes | 133 | 0 | 0% | 127 | 18% | 5 | 7% | |
| Unknown | 8 | | | | | | | |
| CEBPA status | | | | | | | | |
| CEBPA mutant | 49 | 0 | 0% | 43 | 6% | 5 | 7% | 0.806 |
| CEBPA WT | 758 | 6 | 100% | 678 | 94% | 71 | 93% | |
| Unknown | 7 | | | | | | | |
| NPM status | | | | | | | | |
| NPM mutant | 64 | 0 | 0% | 56 | 8% | 7 | 9% | 0.701 |
| NPM WT | 742 | 6 | 100% | 664 | 92% | 69 | 91% | |
| Risk group | | | | | | | | |
| Standard | 385 | 3 | 50% | 350 | 49% | 31 | 41% | 0.410 |
| Low | 307 | 3 | 50% | 261 | 36% | 41 | 54% | 0.010 |
| High | 111 | 0 | 0% | 106 | 15% | 4 | 5% | 0.045 |
| MRD at End of | | | | | | | | |
| Induction 1 | | | | | | | | |
| No | 472 | 4 | 67% | 423 | 58% | 44 | 57% | 0.649 |
| Yes | 212 | 1 | 17% | 194 | 27% | 16 | 21% | |
| Received SCT on | | | | | | | | |
| study | | | | | | | | |
| Yes | 128 | 0 | 0% | 120 | 16% | 7 | 9% | 0.137 |
| No | 689 | 6 | 100% | 610 | 84% | 70 | 91% | |

### CD33 SNPs are Strong Predictors of Clinical Response by Treatment Arm

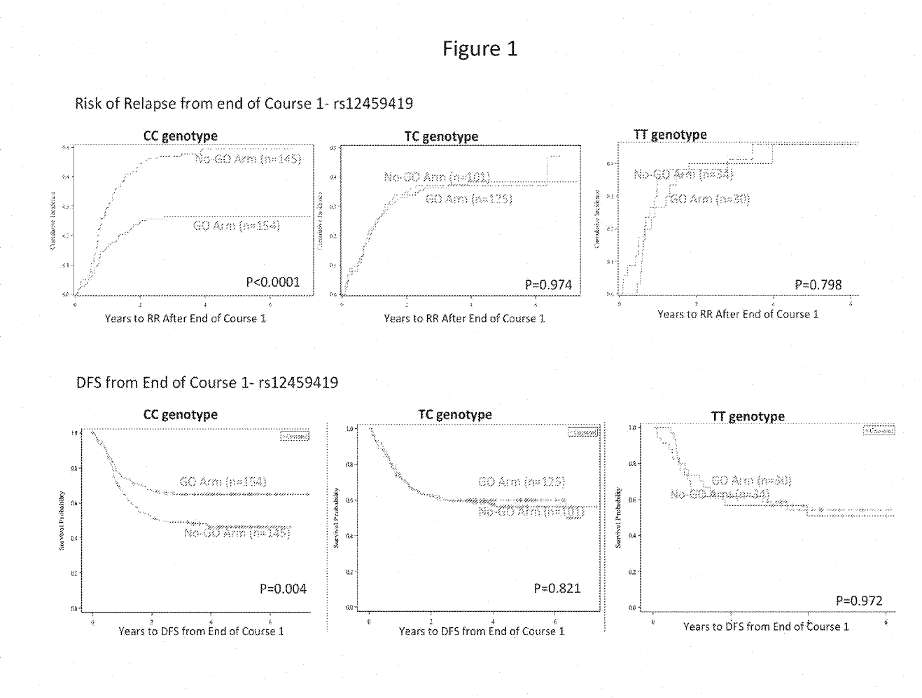

Survival outcome and risk of relapse (RR) analysis indicated that patients with rs12459419 CC genotype had significantly lower RR at 26±7% with addition of GO as compared to 49±9% in No-GO arm (p<0.001) FIG. 1. Similarly, DFS and EFS demonstrated significant improvement in GO vs. No-GO arm (DFS: 65±7% vs. 46±9%; p=0.004; EFS: 54±7% vs. 41±7%; p=0.04). This difference in outcome between GO and No-GO arms was not seen in patients with at least one variant −T allele for rs12459419 (TC and TT genotypes), Table 2.

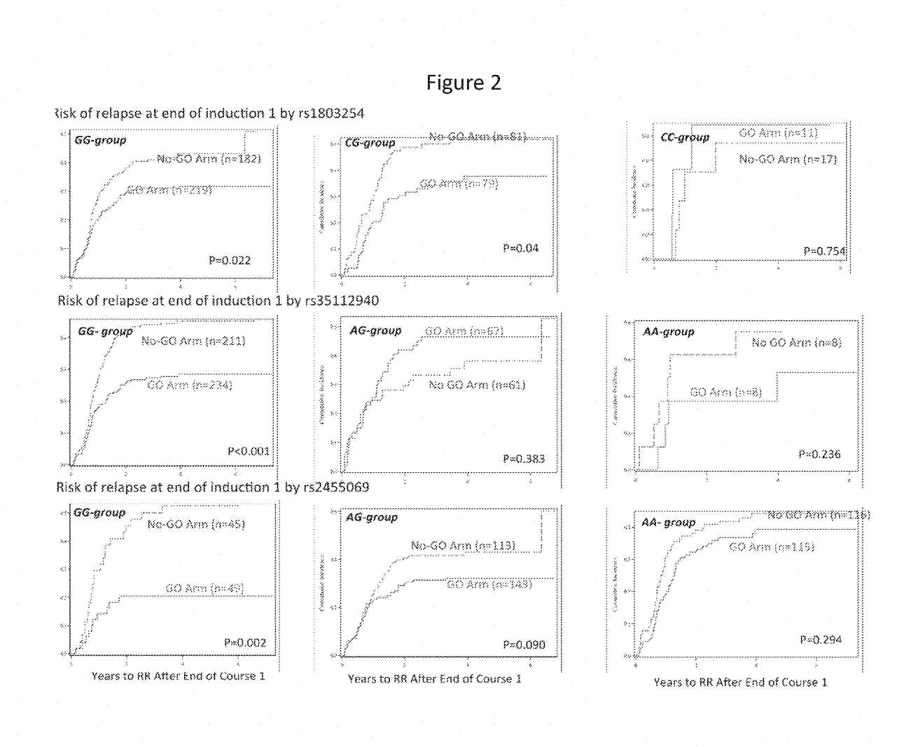

Among other CD33 SNPs: rs1803254, rs35112940 and rs2455609, specific genotype groups demonstrated differences in survival outcome by GO vs. No-GO treatment arms. Table 2 and FIG. 2 provide results for the association analysis between CD33 SNPs and differences in clinical response in patients on GO and No-GO arm. None of the SNPs were associated with overall survival (OS).

TABLE 2
CD33 SNPs and clinical outcome analysis by treatment arm No-GO vs. GO in
patients treated under COG-AAML0531.
rs12459419

| | CC (N = 415) | | | TC (N = 316) | | | TT (N = 85) | | |
|---|---|---|---|---|---|---|---|---|---|
| | No-GO | GO | | No-GO | GO | | No-GO | GO | |
| | (N = 204) | (N = 211) | No-GO | (N = 158) | (N = 158) | No-GO | (N = 46) | (N = 39) | No-GO |
| | % ± | % ± | vs. GO | % ± | % ± | vs. GO | % ± | % ± | vs. GO |
| | 2SE % | 2SE % | p | 2SE % | 2SE % | p | 2SE % | 2SE % | p |
| 5 yr OS from | 58 ± 7% | 66 ± 7% | 0.159 | 69 ± 7% | 64 ± 8% | 0.658 | 70 ± 14% | 69 ± 15% | 0.824 |
| study entry | | | | | | | | | |
| 5 yr EFS from | 41 ± 7% | 54 ± 7% | 0.041 | 53 ± 8% | 51 ± 8% | 0.749 | 44 ± 15% | 49 ± 17% | 0.461 |
| study entry | | | | | | | | | |
| | No-GO | GO | | No-GO | GO | | No-GO | GO | |
| | (N = 145) | (N = 154) | p | (N = 101) | (N = 125) | p | (N = 34) | (N = 30) | p |
| 5 yr DFS from | 46 ± 9% | 65 ± 7% | 0.004 | 60 ± 10% | 56 ± 9% | 0.821 | 54 ± 18% | 51 ± 20% | 0.972 |
| End of Course 1 | | | | | | | | | |
| 5 yr RR from | 49 ± 9% | 26 ± 7% | <0.001 | 37 ± 10% | 38 ± 9% | 0.975 | 46 ± 18% | 46 ± 20% | 0.798 |
| End of Course 1 | | | | | | | | | |
| 5 yr TRM from | 4 ± 3% | 9 ± 5% | 0.135 | 3 ± 3% | 5 ± 4% | 0.496 | — | 3 ± 7% | 0.290 |
| End of Course 1 | | | | | | | | | |

rs3865444

| | CC(N = 412) | | | CA (N = 313) | | | AA (N = 86) | | |
|---|---|---|---|---|---|---|---|---|---|
| | No-GO | GO | | No-GO | GO | | No-GO | GO | |
| | (N = 203) | (N = 209) | No-GO | (N = 156) | (N = 157) | No-GO | (N = 47) | (N = 39) | No-GO |
| | % ± | % ± | vs. GO | % ± | % ± | vs. GO | % ± | % ± | vs. GO |
| | 2SE % | 2SE % | p | 2SE % | 2SE % | p | 2SE % | 2SE % | p |
| 5 yr OS from | 58 ± 7% | 67 ± 7% | 0.134 | 69 ± 7% | 66 ± 8% | 0.773 | 69 ± 14% | 63 ± 20% | 0.768 |
| study entry | | | | | | | | | |
| 5 yr EFS from | 41 ± 7% | 54 ± 7% | 0.034 | 53 ± 8% | 52 ± 8% | 0.618 | 43 ± 15% | 47 ± 17% | 0.558 |
| study entry | | | | | | | | | |
| | No-GO | GO | | No-GO | GO | | No-GO | GO | |
| | (N = 144) | (N = 151) | p | (N = 100) | (N = 124) | p | (N = 34) | (N = 30) | p |
| 5 yr DFS from | 46 ± 9% | 65 ± 8% | 0.003 | 60 ± 10% | 58 ± 9% | 0.954 | 54 ± 18% | 47 ± 20% | 0.792 |
| End of Course 1 | | | | | | | | | |
| 5 yr RR from | 50 ± 9% | 26 ± 7% | <0.001 | 37 ± 10% | 37 ± 9% | 0.750 | 46 ± 18% | 49 ± 20% | 0.964 |
| End of Course 1 | | | | | | | | | |
| 5 yr TRM from | 4 ± 3% | 9 ± 5% | 0.128 | 3 ± 3% | 5 ± 4% | 0.500 | — | 3 ± 7% | 0.290 |
| End of Course 1 | | | | | | | | | |

rs1803254

| | GG (N = 552) | | | CG (N = 224) | | | CC (N = 40) | | |
|---|---|---|---|---|---|---|---|---|---|
| | No-GO | GO | No-GO | No-GO | GO | No-GO | No-GO | GO | No-GO |
| | (N = 268) | (N = 287) | vs. GO | (N = 119) | (N = 219) | vs. GO | (N = 24) | (N = 16) | vs. GO |
| | % ± | % ± | % ± | % ± | % ± | % ± | % ± | % ± | % ± |
| | 2SE % | 2SE % | p | 2SE % | 2SE % | p | 2SE % | 2SE % | p |
| 5 yr OS from | 65 ± 6% | 67 ± 6% | 0.640 | 58 ± 10% | 62 ± 10% | 0.560 | 71 ± 19% | 66 ± 26% | 0.810 |
| study entry | | | | | | | | | |
| 5 yr EFS from | 47 ± 6% | 55 ± 6% | 0.093 | 39 ± 9% | 46 ± 10% | 0.190 | 67 ± 19% | 56 ± 25% | 0.462 |
| study entry | | | | | | | | | |
| | No-GO | GO | | No-GO | GO | | No-GO | GO | |
| | (N = 182) | (N = 287) | p | (N = 81) | (N = 79) | p | (N = 17) | (N = 11) | p |
| 5 yr DFS from | 53 ± 7% | 62 ± 7% | 0.097 | 44 ± 11% | 53 ± 12% | 0.165 | 76 ± 20% | 73 ± 27% | 0.754 |
| End of Course 1 | | | | | | | | | |
| 5 yr RR from | 43 ± 8% | 32 ± 6% | 0.022 | 52 ± 12% | 38 ± 11% | 0.040 | 24 ± 14% | 27 ± 12% | 0.756 |
| End of Course 1 | | | | | | | | | |
| 5 yr TRM from | 3 ± 3% | 6 ± 3% | 0.220 | 4 ± 4% | 9 ± 6% | 0.190 | — | — | — |
| End of Course 1 | | | | | | | | | |

rs35112940

| | GG (N = 620) | | | AG (N = 174) | | | AA (N = 22) | | |
|---|---|---|---|---|---|---|---|---|---|
| | No-GO | GO | | No-GO | GO | | No-GO | GO | |
| | (N = 305) | (N = 315) | No-GO | (N = 91) | (N = 83) | No-GO | (N = 12) | (N = 10) | No-GO |
| | % ± | % ± | vs. GO | % ± | % ± | vs. GO | % ± | % ± | vs. GO |
| | 2SE % | 2SE % | p | 2SE % | 2SE % | p | 2SE % | 2SE % | p |
| 5 yr OS from | 61 ± 6% | 66 ± 6% | 0.360 | 70 ± 10% | 65 ± 13% | 0.820 | 75 ± 25% | 70 ± 30% | 0.850 |
| study entry | | | | | | | | | |
| 5 yr EFS from | 45 ± 6% | 53 ± 5% | 0.091 | 50 ± 11% | 52 ± 11% | 0.671 | — | 45 ± 35% | 0.172 |
| study entry | | | | | | | | | |
| | No-GO | GO | | No-GO | GO | | No-GO | GO | |
| | (N = 211) | (N = 234) | p | (N = 61) | (N = 67) | p | (N = 8) | (N = 8) | p |
| 5 yr DFS from | 51 ± 7% | 63 ± 6% | 0.019 | 59 ± 13% | 52 ± 12% | 0.536 | — | 47 ± 37% | 0.228 |
| End of Course 1 | | | | | | | | | |
| 5 yr RR from | 45 ± 7% | 28 ± 6% | <0.001 | 38 ± 13% | 47 ± 12% | 0.383 | 75 ± 36% | 53 ± 42% | 0.236 |
| End of Course 1 | | | | | | | | | |
| 5 yr TRM from | 3 ± 2% | 8 ± 4% | 0.033 | 3 ± 5% | 1 ± 3% | 0.501 | — | — | — |
| End of Course 1 | | | | | | | | | |

rs2455069

| | AA (N = 312) | | | AG (N = 370) | | | GG (N = 120) | | |
|---|---|---|---|---|---|---|---|---|---|
| | No-GO | GO | | No-GO | GO | | No-GO | GO | |
| | (N = 165) | (N = 147) | No-GO | (N = 177) | (N = 193) | No-GO | (N = 60) | (N = 60) | No-GO |
| | % ± | % ± | vs. GO | % ± | % ± | vs. GO | % ± | % ± | vs. GO |
| | 2SE % | 2SE % | p | 2SE % | 2SE % | p | 2SE % | 2SE % | p |
| 5 yr OS from | 69 ± 4% | 67 ± 8% | 0.700 | 59 ± 8% | 63 ± 8% | 0.244 | 61 ± 13% | 74 ± 12% | 0.247 |
| study entry | | | | | | | | | |
| 5 yr EFS from | 50 ± 8% | 49 ± 8% | 0.876 | 43 ± 8% | 54 ± 7% | 0.015 | 42 ± 13% | 58 ± 13% | 0.216 |
| study entry | | | | | | | | | |
| | No-GO | GO | | No-GO | GO | | No-GO | GO | |
| | (N = 116) | (N = 115) | p | (N = 113) | (N = 143) | p | (N = 45) | (N = 49) | p |
| 5 yr DFS from | 54 ± 10% | 55 ± 10% | 0.608 | 82 ± 10% | 62 ± 8% | 0.084 | 45 ± 15% | 65 ± 14% | 0.105 |
| End of Course 1 | | | | | | | | | |
| 5 yr RR from | 44 ± 10% | 39 ± 9% | 0.294 | 43 ± 10% | 32 ± 8% | 0.090 | 53 ± 16% | 20 ± 12% | 0.002 |
| End of Course 1 | | | | | | | | | |
| 5 yr TRM from | 2 ± 2% | 5 ± 4% | 0.153 | 5 ± 4% | 6 ± 4% | 0.907 | 2 ± 4% | 14 ± 10% | 0.041 |
| End of Course 1 | | | | | | | | | |

rs61736475

| | TT (N = 730) | | | TC (N = 77) | | | CC (N = 6) | | |
|---|---|---|---|---|---|---|---|---|---|
| | No-GO | GO | | No-GO | GO | | No-GO | GO | |
| | (N = 368) | (N = 362) | No-GO | (N = 36) | (N = 41) | No-GO | (N = 3) | (N = 3) | No-GO |
| | % ± | % ± | vs. GO | % ± | % ± | vs. GO | % ± | % ± | vs. GO |
| | 2SE % | 2SE % | p | 2SE % | 2SE % | p | 2SE % | 2SE % | p |
| 5 yr OS from | 65 ± 5% | 66 ± 5% | 0.662 | 52 ± 17% | 61 ± 16% | 0.297 | 67 ± 5% | 67 ± 5% | 0.886 |
| study entry | | | | | | | | | |
| 5 yr EFS from | 46 ± 5% | 53 ± 5% | 0.056 | 41 ± 17% | 55 ± 16% | 0.365 | 67 ± 54% | 0 ± 0% | 0.110 |
| study entry | | | | | | | | | |
| | No-GO | GO | | No-GO | GO | | No-GO | GO | |
| | (N = 252) | (N = 275) | p | (N = 26) | (N = 30) | p | (N = 2) | (N = 2) | p |
| 5 yr DFS from | 52 ± 6% | 60 ± 6% | 0.054 | 57 ± 20% | 69 ± 17% | 0.428 | — | 0 ± 0% | 0.433 |
| End of Course 1 | | | | | | | | | |
| 5 yr RR from | 46 ± 6% | 34 ± 6% | 0.004 | 32 ± 19% | 21 ± 16% | 0.415 | 50 ± | 100 ± | 0.434 |
| End of Course 1 | | | | | | | 100% | 100% | |
| 5 yr TRM from | 2 ± 2% | 6 ± 3% | 0.035 | 12 ± 13% | 10 ± 11% | 0.864 | — | — | — |
| End of Course 1 | | | | | | | | | |

Among other CD33 SNPs, rs1803254 C allele was associated with lower RR in GO vs. No-GO treatment arms (GG: 32±6% vs. 43±8%, p=0.02; CG: 38±11% vs. 52±12%, p=0.04). This difference was not observed in patients homozygous for the low expressing CC genotype (p=0.76). For coding SNP rs35112940, patients homozygous with GG genotype had better outcome by treatment arm (GO vs. No-GO: DFS 63±6% vs. 51.7%, p=0.019; RR 28±6% vs. 45±7%, p<0.001) as compared to patients with at least one low expressing variant allele (AA or AG; p>0.05). Patients homozygous for variant allele (GG) for rs2455609 had significant improvement with RR in GO vs. No-GO arm (20±12 vs. 53±16% p=0.002). Only one SNP-rs35112940 was associated with treatment related mortality (TRM), with GG genotype experiencing higher TRM in GO vs. No-GO arm (14±10% vs. 2±4%, p=0.041). Table 2 provides results for the association analysis between CD33 SNPs and differences in clinical response in patients on GO and No-GO arm. None of the SNPs were associated with OS.

Association of CD33 SNPs with Clinical Outcome within Each Arm

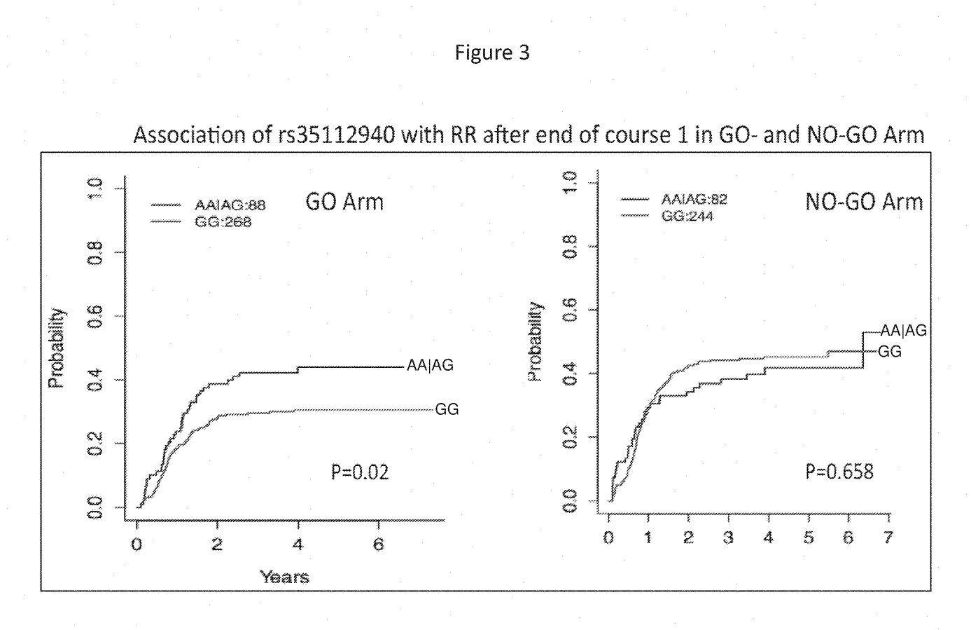

Previous results from AAML03P1 showed rs35112940 GG genotype to be associated with reduced RR and better OS in White patients. Results presented herein are consistent with previous results, in that rs35112940 was associated with lower incidence of RR in GG genotype as compared to patients at least one A allele (GG vs. AG vs. AA; 28±6% vs. 47±12% vs. 53±42% p=0.012, Table 4 and FIG. 3) in GO arm, but not in No-GO arm (p=0.093). Additionally for rs3865444 improvement in RR in patients in GO but not in No-GO arm was observed (GO arm −GG vs. GT vs. TT: 26±7% vs. 37±9% vs. 49±20%, p=0.041, No-GO arm—GG vs. GT vs. TT: 50±9% vs. 37±10% vs. 46±18%, p=0.299).

TABLE 4
Association of CD33 SNPs with clinical outcome within each arm

| CD33 SNP | |
|---|---|
| NO-GO ARM | GO-ARM |

rs35112940

| | AA | | AG | | GG | | | AA | | AG | | GG | | |
|---|---|---|---|---|---|---|---|---|---|---|---|---|---|---|
| | | % ± | | % ± | | % ± | Genotypes | | % ± | | % ± | | % ± | Genotypes |
| | N | 2SE % | N | 2SE % | N | 2SE % | p | N | 2SE % | N | 2SE % | N | 2SE % | p |
| 5 yr RR from | 8 | 75 ± 36% | 61 | 38 ± 13% | 211 | 45 ± 7% | 0.093 | 8 | 53 ± 42% | 67 | 47 ± 12% | 234 | 28 ± 6% | 0.012 |
| End of Course 1 | | | | | | | | | | | | | | |

rs3865444

| | GG | | GT | | TT | | | GG | | GT | | TT | | |
|---|---|---|---|---|---|---|---|---|---|---|---|---|---|---|
| | | % ± | | % ± | | % ± | Genotypes | | % ± | | % ± | | % ± | Genotypes |
| | N | 2SE % | N | 2SE % | N | 2SE % | p | N | 2SE % | N | 2SE % | N | 2SE % | p |
| 5 yr RR from | 144 | 50 ± 9% | 100 | 37 ± 10% | 34 | 46 ± 18% | 0.299 | 151 | 26 ± 7% | 124 | 37 ± 9% | 30 | 49 ± 20% | 0.041 |
| End of Course 1 | | | | | | | | | | | | | | |

rs12459419

| | CC | | TT | | TC | | | CC | | TT | | TC | | |
|---|---|---|---|---|---|---|---|---|---|---|---|---|---|---|
| | | % ± | | % ± | | % ± | Genotypes | | % ± | | % ± | | % ± | Genotypes |
| | N | 2SE % | N | 2SE % | N | 2SE % | p | N | 2SE % | N | 2SE % | N | 2SE % | p |
| 5 yr RR from | 145 | 49 ± 9% | 34 | 46 ± 18% | 101 | 37 ± 10% | 0.290 | 154 | 26 ± 7% | 30 | 46 ± 20% | 125 | 38 ± 9% | 0.059 |
| End of Course 1 | | | | | | | | | | | | | | |

CD33 SNP Rs12459419 is the Strongest Predictor of CD33 Cell Surface Intensity in Patients from COG-AAML0531

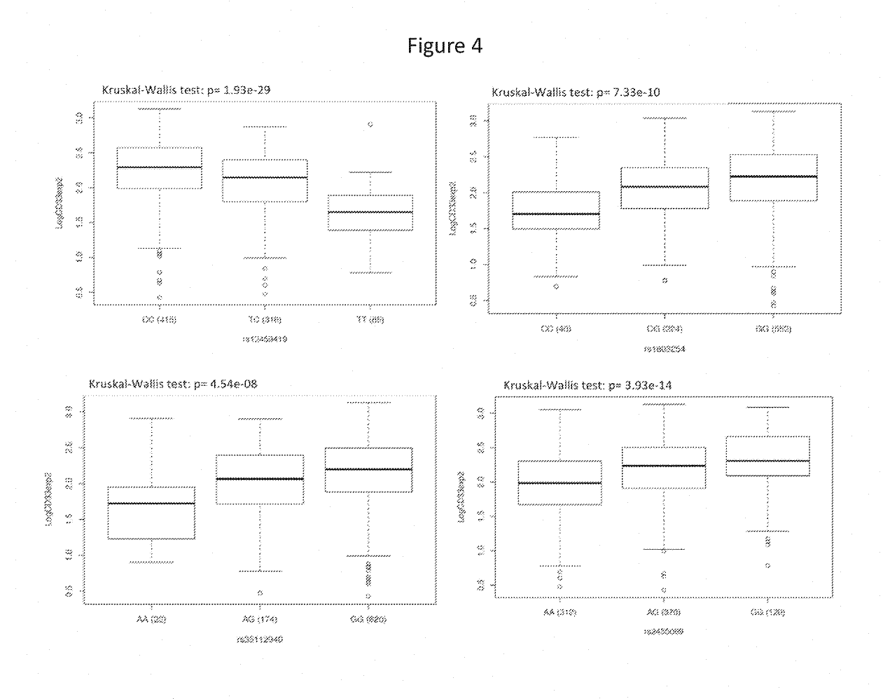

Both genotype and CD33 cell surface intensity (mean fluorescence intensity-MFI) were available in 816 patients (n=408 in No-GO arm and n=408 in GO arm). Presence of variant allele for rs12459419 (and linked promoter SNP rs3865444) was significantly associated with lower CD33 MFI, p=1.9E-29 (Table 3 and FIG. 4). Variant alleles for rs1803254 and rs35112940 were also significantly associated with lower CD33 intensity on leukemic cells (p=7.3E-10 and p=3.9E-14). Coding missense SNP rs2455069 SNP was associated with higher CD33 expression (p=4.5E-8).

TABLE 3
Association of CD33 SNPs with CD33 cell surface intensity on Leukemic cells

| | | | MAF | | Direction of | | |
|---|---|---|---|---|---|---|---|
| | | Allele | Whites/ | P value | Association of | Genotype | CD33 Expression |
| CD33 SNP | SNP type | Change | Blacks | (Kruskal Wallis) | variant allele | distribution | Median (Range) |
| rs32459419 (LD | Missense/ | C > T | 0.32/ | p = 1.93e−29 | Low | CC (n = 415) | 195 (2.65-1351) |
| with promoter | Va14Val | (C > A) | 0.13 | | | CT (n = 316) | 140.84 (3-748.47) |
| SNP rs3865444) | | | | | | TT (n = 85) | 44.5 (6-813) |
| rs1803254 | 3′UTR | G > C | 0.15/ | p = 7.32e−10 | Low | GG (n = 552) | 50.76 (5-.584) |
| | | | 0.32 | | | CG (n = 224) | 121.67 (6-1066) |
| | | | | | | CC (n = 40) | 168.17 (2.66-1351) |
| rs2455069 | Missense | A > G | 0.41/ | p = 3.93e−14 | High | AA (n = 312) | 96.9 (3-1130.6) |
| | Arg60Gly | | 0.40 | | | AG (n = 370) | 172 (3-2.68-1351) |
| | | | | | | GG (n = 120) | 204.15 (6.12-1225.8) |
| rs35112940 | Missens | G > A | 0.15/ | p = 4.54e−8 | Low | GG (n = 620) | 157.57 (2.68-1351) |
| | Arg304Gly | | 0.07 | | | AG (n = 174) | 116.05 (3-797.2) |
| | | | | | | AA (n = 22) | 52.69 (8-813) |
| rs63736475 | Missens | T > C | 0.028/ | p = 0.129 | NA | TT (n = 730) | 146.97 (2.68-1351) |
| | Ser305Pro | | 0.27 | | | TC (n = 77) | 111 (5-1025.07) |
| | | | | | | CC (n = 6) | 381.24 (29.39-119.5) |

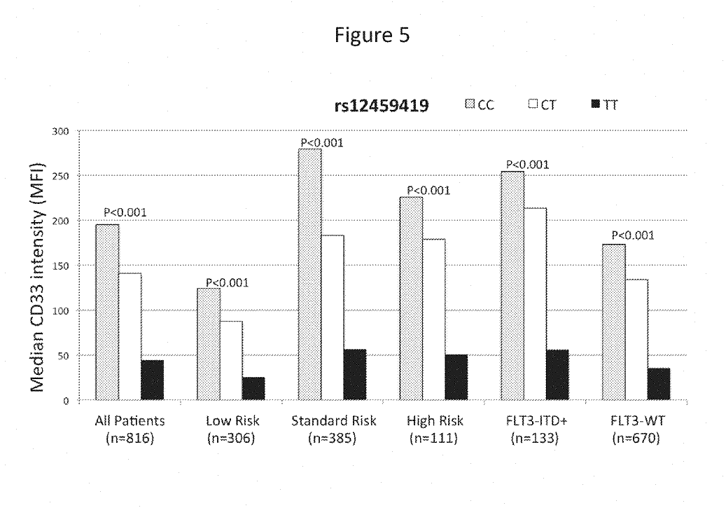

Analysis within each arm, risk group as well as by FLT3-ITD status was consistent with results from the total study cohort (Table 5, and FIG. 5). Since frequency of the selected CD33 SNPs within risk groups or by FLT3-ITD status was not different, the impact of CD33 SNPs on its cell surface intensity appears to be independent of risk group characteristics. Previous studies (Pollard J A et al., Journal of clinical oncology: official journal of the American Society of Clinical Oncology, 2016; 34(7):747-55; Pollard et al., Blood 2012; 119(16):3705-11) have utilized quartile system to classify patients based on CD33 cell surface expression; consistent results were obtained here with association of SNPs with quartiles (Table 6).

TABLE 5
Association of CD33 SNPs expression within different subgroups of AML patients
rs12459419

| | | | | | | | | | | | Genotype |
|---|---|---|---|---|---|---|---|---|---|---|---|
| | | | | | | | | | | | Com- |
| | Total | C (N = 415) | | | T (N = 85) | | | TC (N = 316) | | | parison |
| SNPs | N | N | Median | Range | N | Median | Range | N | Median | Range | p |
| CD33 Expression, NoGo | 408 | 204 | 205.15 | (4.27-1077.7) | 46 | 33.4 | (6-813) | 158 | 126.47 | (3-748.47) | <0.001 |
| CD33 Expression, Go | 408 | 211 | 175 | (2.68-1351) | 39 | 58.62 | (6.84-134.47) | 158 | 158.77 | (4-713) | <0.001 |
| CD33 Expression, | 385 | 192 | 279.26 | (2.68-1351) | 44 | 56.49 | (6-813) | 149 | 183 | (3-713) | <0.001 |
| Standard Risk | | | | | | | | | | | |
| CD33 Expression, Low | 306 | 159 | 124 | (11.23-876) | 28 | 25.34 | (6.84-91) | 119 | 87.31 | (5-545.03) | <0.001 |
| Risk | | | | | | | | | | | |
| CD33 Expression, High | 111 | 55 | 225.74 | (4.27-1225.87) | 11 | 50.55 | (12-139.47) | 45 | 178.73 | (9.83-748.47) | <0.001 |
| Risk | | | | | | | | | | | |
| CD33 Expression, FLT+ | 133 | 66 | 254.26 | (4.27-1225.87) | 15 | 55.9 | (12-165.1) | 52 | 213.28 | (25-706) | <0.001 |
| CD33 Expression, FLT3− | 670 | 341 | 173 | (2.68-1351) | 68 | 35.48 | (6-165.14) | 261 | 133.85 | (3-748.47) | <0.001 |

rs3865444

| | | | | | | | | | | | Genotype |
|---|---|---|---|---|---|---|---|---|---|---|---|
| | | | | | | | | | | | Com- |
| | Total | G (N = 412) | | | GT (N = 313) | | | T (N = 86) | | | parison |
| SNPs | N | N | Median | Range | N | Median | Range | N | Median | Range | p |
| CD33 Expression, All | 811 | 412 | 193.16 | (2.68-1351) | 313 | 141 | (3-748.47) | 86 | 46.25 | (6-813) | <0.001 |
| CD33 Expression, NoGo | 406 | 203 | 203.29 | (4.27-1077.72) | 156 | 126.83 | (3-748.47) | 47 | 34.22 | (6-813) | <0.001 |
| CD33 Expression, Go | 405 | 209 | 173 | (2.68-1351) | 157 | 156 | (4-713) | 39 | 58.62 | (6.84-439) | <0.001 |
| CD33 Expression, | 383 | 191 | 273.92 | (2.68-1351) | 147 | 183 | (3-713) | 45 | 58.62 | (6-813) | <0.001 |
| Standard Risk | | | | | | | | | | | |
| CD33 Expression, Low | 303 | 157 | 123 | (11.23-876) | 118 | 89.86 | (5-545.03) | 28 | 25.34 | (6.84-91) | <0.001 |
| Risk | | | | | | | | | | | |
| CD33 Expression, High | 111 | 55 | 225.74 | (4.27-1225.87) | 45 | 178.73 | (9.83-748.47) | 11 | 50.55 | (12-139.47) | <0.001 |
| Risk | | | | | | | | | | | |
| CD33 Expression, FLT+ | 133 | 66 | 254.26 | (4.27-1225.87) | 52 | 213.275 | (25-706) | 15 | 55.9 | (12-165.1) | <0.001 |
| CD33 Expression, FLT3− | 666 | 338 | 171 | (2.68-1351) | 259 | 133.85 | (3-748.47) | 69 | 35.95 | (6-439) | <0.001 |

rs1803254

| | | | | | | | | | | | Genotype |
|---|---|---|---|---|---|---|---|---|---|---|---|
| | | | | | | | | | | | Com- |
| | Total | C (N = 40) | | | CG (N = 224) | | | G (N = 552) | | | parison |
| SNPs | N | N | Median | Range | N | Median | Range | N | Median | Range | p |
| CD33 Expression, NoGo | 408 | 24 | 50.76 | (20-584) | 119 | 117 | (6-748.47) | 265 | 163 | (3-1077.72) | <0.001 |
| CD33 Expression, Go | 408 | 16 | 56.26 | (5-318.8) | 105 | 121.89 | (10-1086) | 287 | 171 | (2.68-1351) | <0.001 |
| CD33 Expression, | 385 | 21 | 80 | (7.6-584) | 104 | 174.19 | (6-1086) | 260 | 226.5 | (2.68-1351) | <0.001 |
| Standard Risk | | | | | | | | | | | |
| CD33 Expression, Low | 306 | 16 | 42.98 | (5-153.88) | 81 | 87.31 | (10-455.24) | 209 | 113.26 | (9.34-876) | <0.001 |
| Risk | | | | | | | | | | | |
| CD33 Expression, High | 111 | 3 | 50.55 | (32.58-55.90) | 36 | 128.24 | (9.83-748.47) | 72 | 215.31 | (4.27-1225.87) | 0.005 |
| Risk | | | | | | | | | | | |
| CD33 Expression, FLT+ | 133 | 3 | 50.55 | (32.58-55.9) | 41 | 146.01 | (10.51-478.64) | 89 | 239.25 | (4.27-1225.87) | 0.001 |
| CD33 Expression, FLT3− | 670 | 37 | 50.96 | (5-584) | 180 | 119.28 | (6-1086) | 453 | 146.94 | (2.68-1351) | <0.001 |

rs2455069

| | | | | | | | | | | | Genotype |
|---|---|---|---|---|---|---|---|---|---|---|---|
| | | | | | | | | | | | Com- |
| | Total | A (N = 312) | | | AG (N = 370) | | | G (N = 120) | | | parison |
| SNPs | N | N | Median | Range | N | Median | Range | N | Median | Range | p |
| CD33 Expression, NoGo | 402 | 165 | 37 | (3-911) | 177 | 164 | (4.27-1077.72) | 60 | 220.26 | (6.12-900) | <0.001 |
| CD33 Expression, Go | 400 | 147 | 117 | (4-1130.6) | 193 | 174.12 | (2.68-1351) | 60 | 186.32 | 12.25-1225.87 | <0.001 |
| CD33 Expression, | 317 | 146 | 143.5 | (3-1130.6) | 184 | 225 | (2.68-1351) | 47 | 324.09 | (6.12-1086) | <0.001 |
| Standard Risk | | | | | | | | | | | |
| CD33 Expression, Low | 301 | 115 | 63.42 | (5-545.03) | 135 | 99.75 | (11.23-876) | 51 | 147.11 | (19.22-698.08) | <0.001 |
| Risk | | | | | | | | | | | |
| CD33 Expression, High | 110 | 45 | 114.11 | (12-628.93) | 45 | 207 | (4.27-860.09) | 20 | 358.05 | 12.25-1225.87 | 0.003 |
| Risk | | | | | | | | | | | |
| CD33 Expression, FLT+ | 131 | 55 | 144.17 | (12-628.93) | 49 | 207.24 | (4.27-711.68) | 27 | 362.73 | 12.25-1225.87 | <0.001 |
| CD33 Expression, FLT3− | 658 | 253 | 89.44 | (3-1130.6) | 316 | 160.77 | (2.68-1351) | 89 | 171 | (6.12-1086) | <0.001 |

rs35112940

| | | | | | | | | | | | Genotype |
|---|---|---|---|---|---|---|---|---|---|---|---|
| | | | | | | | | | | | Com- |
| | Total | A (N = 22) | | | AG (N = 174) | | | G (N = 620) | | | parison |
| SNPs | N | N | Median | Range | N | Median | Range | N | Median | Range | p |
| CD33 Expression, NoGo | 408 | 12 | 22.5 | (8-813) | 91 | 85 | (3-706) | 305 | 158.38 | (4.27-1077.72) | <0.001 |
| CD33 Expression, Go | 408 | 10 | 62.21 | (16-129.45) | 83 | 148 | (10-797.2) | 315 | 157.14 | (2.68-1351) | 0.011 |
| CD33 Expression, | 385 | 12 | 56.705 | (8-813) | 86 | 156.94 | (3-797.2) | 287 | 218.45 | (2.68-1351) | <0.001 |
| Standard Risk | | | | | | | | | | | |
| CD33 Expression, Low | 306 | 7 | 23 | (9.34-82.38) | 61 | 77 | (10-355.74) | 238 | 107.12 | (5-876) | <0.001 |
| Risk | | | | | | | | | | | |
| CD33 Expression, High | 111 | 2 | 83.135 | (52.97-113.3) | 24 | 147.33 | (12-459.02) | 85 | 207.01 | (4.27-1225.87) | 0.199 |
| Risk | | | | | | | | | | | |
| CD33 Expression, FLT+ | 133 | 4 | 71.24 | (40-107.65) | 29 | 178.73 | (12-797.2) | 100 | 235.77 | (4.27-1225.87) | 0.068 |
| CD33 Expression, FLT3− | 670 | 16 | 27.01 | (8-129.45) | 144 | 97.08 | (3-713) | 510 | 147.06 | (2.68-1351) | <0.001 |

rs61736475

| | | | | | | | | | | | Genotype |
|---|---|---|---|---|---|---|---|---|---|---|---|
| | | | | | | | | | | | Com- |
| | Total | C (N = 6) | | | T (N = 730) | | | TC (N = 77) | | | parison |
| SNPs | N | N | Median | Range | N | Median | Range | N | Median | Range | p |
| CD33 Expression, NoGo | 407 | 3 | 86 | (29.39-318.47) | 368 | 142 | (3-1077.72) | 36 | 138.69 | 13.49-1025.07 | 0.866 |
| CD33 Expression, Go | 406 | 3 | 1086 | (444-1119.5) | 362 | 155 | (2.68-1351) | 41 | 94.13 | (5-543.81) | 0.004 |
| CD33 Expression, | 384 | 3 | 1086 | (444-1119.5) | 350 | 199 | (2.68-1351) | 31 | 186.87 | 13.49-1025.07 | 0.027 |
| Standard Risk | | | | | | | | | | | |
| CD33 Expression, Low | 305 | 3 | 86 | (29.39-318.47) | 261 | 99.75 | (6.84-698.08) | 41 | 85 | (5-876) | 0.575 |
| Risk | | | | | | | | | | | |
| CD33 Expression, High | 110 | 0 | — | — | 106 | 182.915 | (4.27-1225.87) | 4 | 217.125 | (67-436.73) | 0.737 |
| Risk | | | | | | | | | | | |
| CD33 Expression, FLT+ | 132 | 0 | — | — | 127 | 207.24 | (4.27-1225.87) | 5 | 347 | (85-436.76) | 0.478 |
| CD33 Expression, FLT3− | 668 | 6 | 381.235 | (29.39-1119.5) | 591 | 136.84 | (2.68-1351) | 71 | 100.7 | (5-1025.07) | 0.119 |

TABLE 6
Association of CD33 SNPs with CD33 expression Quartiles
rs12459419

| | | CD33 | | CD33 | | CD33 | | CD33 | | | |
|---|---|---|---|---|---|---|---|---|---|---|---|
| | | Q1 | | Q2 | | Q3 | | Q4 | | | |
| | | (N = | | (N = | | (N = | | (N = | | Q1 vs. Q2 vs. | |
| | Total | 207) | | 204) | | 203) | | 203) | | Q3 vs. Q4 | Q1 vs. Q2-4 |
| | N | N | % | N | % | N | % | N | % | p | p |
| CC | 415 | 62 | 30% | 103 | 50% | 105 | 52% | 145 | 71% | <0.001 | <0.001 |
| TT | 85 | 59 | 29% | 23 | 11% | 2 | 1% | 1 | 0% | <0.001 | <0.001 |
| TC | 316 | 85 | 41% | 78 | 38% | 96 | 47% | 57 | 28% | 0.001 | 0.415 |

rs3865444

| | | CD33 | | CD33 | | CD33 | | CD33 | | | |
|---|---|---|---|---|---|---|---|---|---|---|---|
| | | Q1 | | Q2 | | Q3 | | Q4 | | | |
| | | (N = | | (N = | | (N = | | (N = | | Q1 vs. Q2 vs. | |
| | Total | 205) | | 204) | | 201) | | 201) | | Q3 vs. Q4 | Q1 vs. Q2-4 |
| | N | N | % | N | % | N | % | N | % | p | p |
| GG | 412 | 63 | 31% | 103 | 50% | 103 | 51% | 143 | 71% | <0.001 | <0.001 |
| GT | 313 | 83 | 40% | 78 | 38% | 96 | 48% | 56 | 28% | 0.001 | 0.519 |
| TT | 86 | 59 | 29% | 23 | 11% | 2 | 1% | 2 | 1% | <0.001 | <0.001 |

rs1803254

| | | CD33 | | CD33 | | CD33 | | CD33 | | | |
|---|---|---|---|---|---|---|---|---|---|---|---|
| | | Q1 | | Q2 | | Q3 | | Q4 | | | |
| | | (N = | | (N = | | (N = | | (N = | | Q1 vs. Q2 vs. | |
| | Total | 206) | | 204) | | 203) | | 203) | | Q3 vs. Q4 | Q1 vs. Q2-4 |
| SNPs | N | N | % | N | % | N | % | N | % | p | p |
| CC | 40 | 23 | 11% | 10 | 5% | 4 | 2% | 3 | 1% | <0.001 | <0.001 |
| CG | 224 | 64 | 31% | 61 | 30% | 66 | 33% | 33 | 16% | 0.001 | 0.179 |
| GG | 552 | 119 | 58% | 133 | 65% | 133 | 66% | 167 | 82% | <0.001 | 0.001 |

rs2455069

| | | CD33 | | CD33 | | CD33 | | CD33 | | | |
|---|---|---|---|---|---|---|---|---|---|---|---|
| | | Q1 | | Q2 | | Q3 | | Q4 | | | |
| | | (N = | | (N = | | (N = | | (N = | | Q1 vs. Q2 vs. | |
| | Total | 203) | | 200) | | 198) | | 201) | | Q3 vs. Q4 | Q1 vs. Q2-4 |
| | N | N | % | N | % | N | % | N | % | p | p |
| AA | 312 | 117 | 58% | 80 | 40% | 66 | 33% | 49 | 24% | <0.001 | <0.001 |
| AG | 370 | 75 | 37% | 89 | 45% | 102 | 52% | 104 | 52% | 0.008 | 0.002 |
| GG | 120 | 11 | 5% | 31 | 16% | 30 | 15% | 48 | 24% | <0.001 | <0.001 |

rs35112940

| | | CD33 | | CD33 | | CD33 | | CD33 | | | |
|---|---|---|---|---|---|---|---|---|---|---|---|
| | | Q1 | | Q2 | | Q3 | | Q4 | | | |
| | | (N = | | (N = | | (N = | | (N = | | Q1 vs. Q2 vs. | |
| | Total | 206) | | 204) | | 203) | | 203) | | Q3 vs. Q4 | Q1 vs. Q2-4 |
| | N | N | % | N | % | N | % | N | % | p | p |
| AA | 22 | 15 | 7% | 6 | 3% | 0 | 0% | 1 | 0% | <0.001 | <0.001 |
| AG | 174 | 57 | 28% | 44 | 22% | 42 | 21% | 31 | 15% | 0.024 | 0.010 |
| GG | 620 | 134 | 65% | 154 | 75% | 161 | 79% | 171 | 84% | <0.001 | <0.001 |

rs61736475

| | | CD33 | | CD33 | | CD33 | | CD33 | | | |
|---|---|---|---|---|---|---|---|---|---|---|---|
| | | Q1 | | Q2 | | Q3 | | Q4 | | | |
| | | (N = | | (N = | | (N = | | (N = | | Q1 vs. Q2 vs. | |
| | Total | 207) | | 203) | | 201) | | 202) | | Q3 vs. Q4 | Q1 vs. Q2-4 |
| | N | N | % | N | % | N | % | N | % | p | p |
| CC | 6 | 1 | 0% | 1 | 0% | 0 | 0% | 4 | 2% | 0.107 | 1.000 |
| TT | 730 | 182 | 88% | 183 | 90% | 183 | 91% | 182 | 90% | 0.757 | 0.304 |
| TC | 77 | 24 | 12% | 19 | 9% | 18 | 9% | 16 | 8% | 0.633 | 0.227 |

Rs12459419 C>T is Associated with Higher Levels of Exon 2 Skipped Spliced Isoform and Production of Shorter CD33 Isoform Lacking IgV Domain

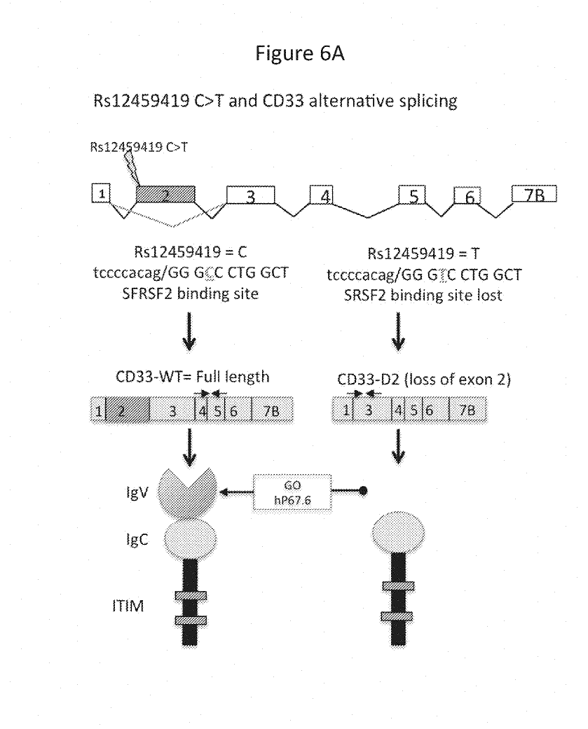

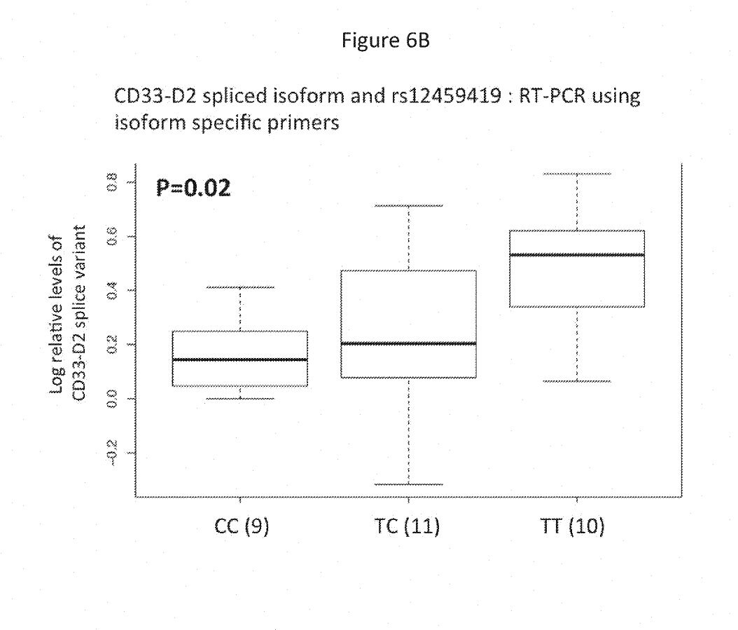

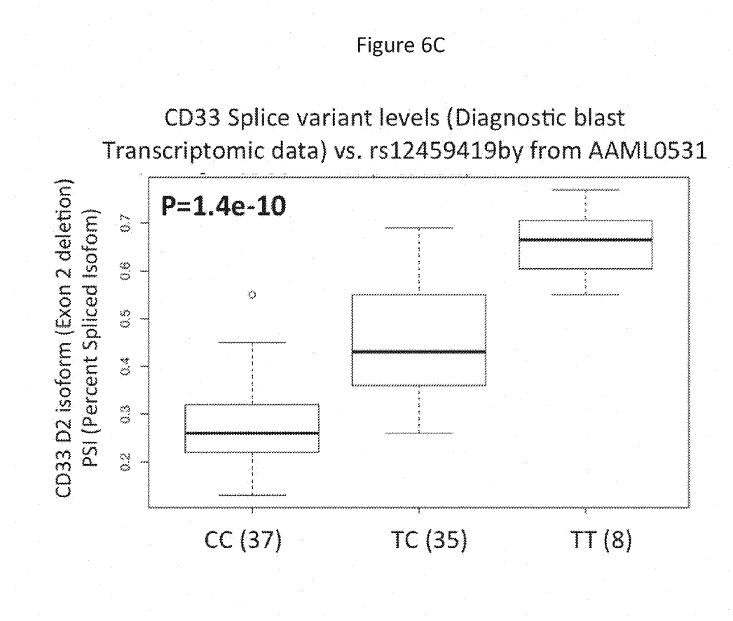

Although mechanisms underlying the functional consequences of CD33 coding and regulatory SNPs are yet unknown, an alternatively spliced isoform of CD33 skipping exon 2 (D2 isoform) and thus lacking IgV domain has been reported previously (20-22). hP67.6-CD33 antibody as well as GO recognizes IgV region of CD33; thus, loss of this domain can not only interfere with detection of total CD33 (only detects full length but not short form lacking IgV domain (Perez-Oliva et al., Glycobiology, 2011; 21(6):757-70), but can influence cellular sensitivity to GO, FIG. 6A. CD33 SNP rs12459419 in exon 2 is present within 4 bp of exonic junction, and C>T results in Ala>Val change at codon 14. This SNP also resides within and alters exonic splicing enhancer binding site for SRSF2 (http://rulai.cshl.edu/cgi-bin/tools/ESE3/esefinder.cgi?process=home) and has been recently associated with exon 2 deletion (Raj et al., Hum Mol Genet, 2014; 23(10):2729-36; et al., Hum Mol Genet, 2015; 24(12):3557-70; Malik et al., J Neurosci, 2013; 33(33): 13320-5). This alternatively spliced, shorter isoform of CD33 missing exon-2 (D2 isoform) lacks the IgV domain. The exon-2 encoding IgV domain is known to be the epitope for hP67.6-CD33 antibody, which is used for diagnostic immunophenotyping as well as for the antibody that is conjugated to calicheamicin in GO. As a result, loss of this domain would not only interfere with detection of total CD33, but can impact binding, internalization and thus clinical efficacy of GO, FIG. 6A.

We inquired whether minor allele is associated with alternate splicing. We used transcriptome sequencing data available from 80 patients from AAML0531 to correlate rs12459419 genotype with CD33 isoforms. We demonstrate that rs12459419 variant T allele is significantly associated with increased levels of alternatively spliced CD33-D2 isoform lacking exon2 (p=1.4E-10; FIG. 6B).

Quantitative RT-PCR of RNA from diagnostic leukemic blasts from a completely different set of 30 AAML0531 samples (CC=9, CT=11, and TT=10) using isoform specific primers shown in FIG. 1 showed significant association of T allele with increased levels isoform lacking exon 2 (FIG. 6C; p=0.02). No association of total CD33 transcript levels with rs12459419 was observed within this cohort (p>0.05) for both the sets investigated above (RNA-seq and RT-PCR).

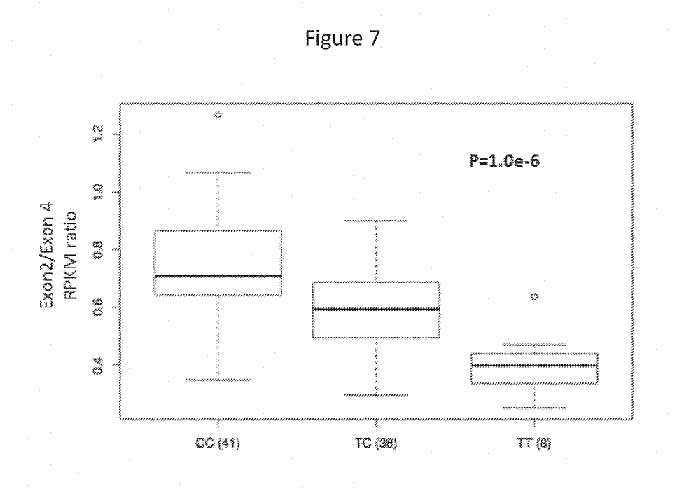

Using transcriptome sequencing data we also show that rs12459419 T allele is associated with lower levels of exon 2 relative to levels of exon 4 which is constitutive as reflected by lower exon 2:exon 4 ratio in patients with CT or TT genotype in FIG. 7. These results are consistent with lower cell surface intensity of CD33 in leukemic cells from patients with TT genotype as detected by current method that uses hP67.6.

### CD33SNP_SCORE Score is Predictive of Outcome by Arm

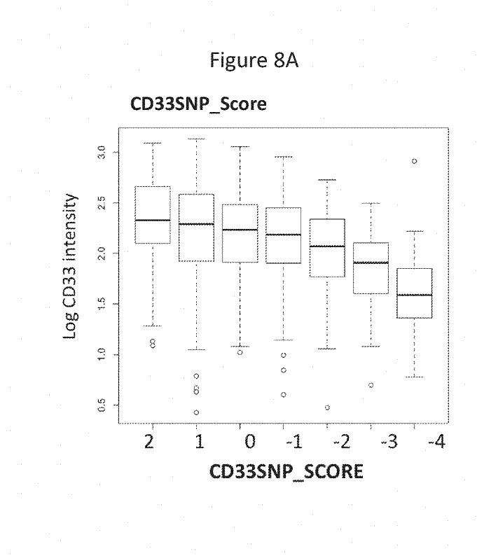

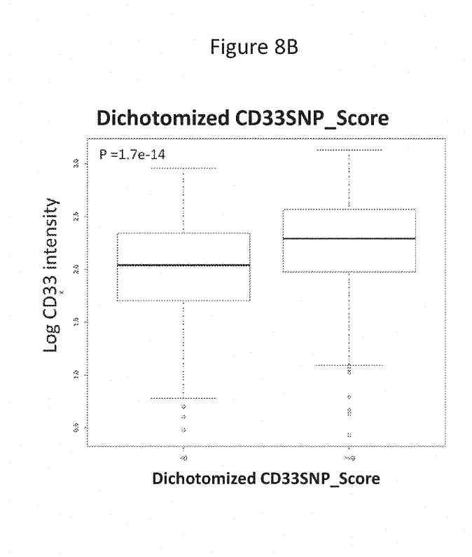

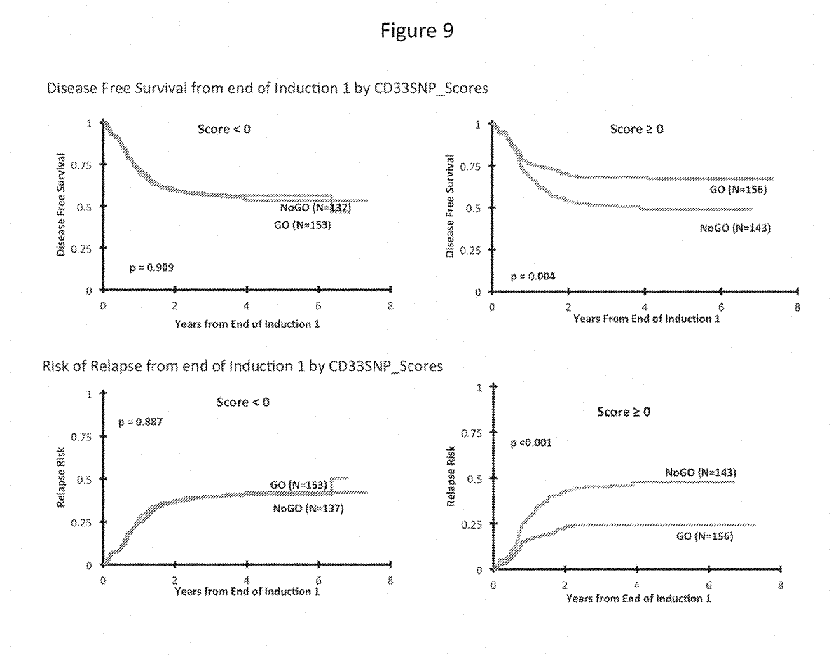

Since 5 CD33 SNPs: rs12459419, rs3865444, rs1803254, rs2455069 and rs35112940 were associated with clinical outcome by arm and CD33 cell surface expression, and other than rs12459419 and rs3865444, none of the SNPs were in LD, a scoring system referred to herein as “CD33SNP_Score” (ranging from 2 to −4) was created for each patient by adding the directional genotype scores of each SNP. Further CD33SNP_Scores were combined to generate a dichotomized score: Scores of ≥0 and <0 and demonstrated association with CD33 cell surface expression (FIG. 8). Patients with scores of ≥0 demonstrated better outcome with DFS being 49±9% vs. 67±8% (p=0.004); and RR being 24±7% vs. 48±9% (p<0.001) in NO-GO vs. GO arm, Table 7 and FIG. 9. Analysis within each risk group showed significant improvement in outcome in Low-Risk group patients with score ≥0, DFS of 82±10% vs. 60±12% (p=0.005) and RR, 10±7% vs. 36±17% (p=0.001) in GO vs. No-GO arms. Similarly in standard risk group patients with score ≥0, RR was significantly lower in GO vs. No-Go arm (36±12% vs. 57±13%; p=0.030); in High-Risk group we observed a trend towards lower RR 38±25% vs. 70±32% in GO arm vs. No-GO (p=0.073); Table 7. The results in high-risk group category might be confounded by sample size. Conversely patients with score of <0 showed no benefit from addition of GO with DFS and RR being similar in both arms (p>0.05).

Rs12459419 was the primary driver of the CD33_SNP score with ˜95% patients with score of ≥0 being CC genotype (no one with TT genotype) and around 94% of patients with score <0 having at least one T allele (with CT or TT genotype, indicating that single CD33 SNP-rs12459419 could be used as a biomarker to determine patients' response to CD33 targeted agents.

TABLE 7
Association of dichotomized CD33-3SNP score with clinical outcome by GO or
No-GO arm in all patients and by risk group
All Patients

| | Score < 0 (N = 405) | | | | | Score ≥ 0 (N = 412) | | | | |
|---|---|---|---|---|---|---|---|---|---|---|
| | NoGO | | | | NoGO | NoGO | | | | NoGO |
| | (N = 210) | | GO (N = 195) | | vs. GO | (N = 198) | | GO (N = 214) | | vs. GO |
| | N | | N | | p | N | | N | | p |
| 5 yr OS from | 210 | 67 ± 7% | 195 | 63 ± 7% | 0.696 | 198 | 60 ± 7% | 214 | 69 ± 7% | 0.174 |
| study entry | | | | | | | | | | |
| 5 yr EFS from | 210 | 49 ± 7% | 195 | 49 ± 7% | 0.579 | 198 | 43 ± 7% | 214 | 56 ± 7% | 0.030 |
| study entry | | | | | | | | | | |
| 5 yr DFS from | 137 | 56 ± 9% | 153 | 53 ± 8% | 0.909 | 143 | 49 ± 9% | 156 | 67 ± 8% | 0.004 |
| End of Course 1 | | | | | | | | | | |
| 5 yr RR from | 137 | 41 ± 9% | 153 | 42 ± 8% | 0.887 | 143 | 48 ± 9% | 156 | 24 ± 7% | <0.001 |
| End of Course 1 | | | | | | | | | | |

Low Risk

| | Score < 0 (N = 143) | | | | | Score ≥ 0 (N = 164) | | | | |
|---|---|---|---|---|---|---|---|---|---|---|
| | NoGO | | | | NoGO | NoGO | | | | NoGO |
| | (N = 76) | | GO (N = 67) | | vs. GO | (N = 77) | | GO (N = 87) | | vs. GO |
| | N | | N | | p | N | | N | | p |
| 5 yr OS from | 76 | 87 ± 8% | 67 | 76 ± 11% | 0.123 | 77 | 75 ± 10% | 87 | 89 ± 7% | 0.036 |
| study entry | | | | | | | | | | |
| 5 yr EFS from | 76 | 69 ± 11% | 67 | 66 ± 12% | 0.574 | 77 | 57 ± 12% | 87 | 79 ± 9% | 0.003 |
| study entry | | | | | | | | | | |
| 5 yr DFS from | 58 | 67 ± 13% | 57 | 68 ± 12% | 0.981 | 67 | 60 ± 12% | 71 | 82 ± 10% | 0.005 |
| End of Course 1 | | | | | | | | | | |
| 5 yr RR from | 58 | 30 ± 12% | 57 | 25 ± 12% | 0.620 | 67 | 36 ± 12% | 71 | 10 ± 7% | 0.001 |
| End of Course 1 | | | | | | | | | | |

Standard Risk

| | Score < 0 (N = 200) | | | | | Score ≥ 0 (N = 185) | | | | |
|---|---|---|---|---|---|---|---|---|---|---|
| | NoGO | | | | NoGO | NoGO | | | | NoGO |
| | (N = 101) | | GO (N = 99) | | vs. GO | (N = 94) | | GO (N = 91) | | vs. GO |
| | N | | N | | p | N | | N | | p |
| 5 yr OS from | 101 | 59 ± 10% | 99 | 57 ± 11% | 0.675 | 94 | 52 ± 11% | 91 | 60 ± 10% | 0.620 |
| study entry | | | | | | | | | | |
| 5 yr EFS from | 101 | 39 ± 10% | 99 | 41 ± 10% | 0.319 | 94 | 35 ± 10% | 91 | 47 ± 11% | 0.278 |
| study entry | | | | | | | | | | |
| 5 yr DFS from | 58 | 49 ± 13% | 77 | 42 ± 12% | 0.840 | 64 | 40 ± 12% | 65 | 58 ± 12% | 0.073 |
| End of Course 1 | | | | | | | | | | |
| 5 yr RR from | 58 | 49 ± 13% | 77 | 55 ± 12% | 0.898 | 64 | 57 ± 13% | 65 | 36 ± 12% | 0.030 |
| End of Course 1 | | | | | | | | | | |

High Risk

| | Score < 0 (N = 57) | | | | | Score ≥ 0 (N = 54) | | | | |
|---|---|---|---|---|---|---|---|---|---|---|
| | NoGO | | | | NoGO | NoGO | | | | NoGO |
| | (N = 30) | | GO (N = 27) | | vs. GO | (N = 24) | | GO (N = 30) | | vs. GO |
| | N | | N | | p | N | | N | | p |
| 5 yr OS from | 30 | 38 ± 19% | 27 | 54 ± 20% | 0.251 | 24 | 46 ± 23% | 30 | 45 ± 19% | 0.993 |
| study entry | | | | | | | | | | |
| 5 yr EFS from | 30 | 28 ± 17% | 27 | 41 ± 19% | 0.197 | 24 | 29 ± 19% | 30 | 26 ± 16% | 0.449 |
| study entry | | | | | | | | | | |
| 5 yr DFS from | 18 | 40 ± 25% | 18 | 56 ± 23% | 0.232 | 10 | 30 ± 29% | 16 | 44 ± 25% | 0.496 |
| End of Course 1 | | | | | | | | | | |
| 5 yr RR from | 18 | 54 ± 26% | 18 | 39 ± 24% | 0.293 | 10 | 70 ± 32% | 16 | 38 ± 25% | 0.090 |
| End of Course 1 | | | | | | | | | | |

### DISCUSSION

Here, common CD33 SNPs are reported that are significant predictors of clinical response to GO-based therapy in pediatric AML patients. GO is an anti-CD33 targeted antibody linked to cytotoxin calicheamicin and has shown promising results in AML. Results from meta-analysis studies further suggest GO provides survival benefits in patients with favorable cytogenetics (9,10). In pediatric AML, results from two Children's Oncology group trials: AAML03P1 and AAML0531 demonstrated improved outcome with addition of GO to chemotherapy (11,12).

GO targets CD33 and higher CD33 expression levels have been previously correlated with in vitro GO chemosensitivity, indicating the significance of CD33 expression levels on GO response (25-27).

Results from COG AAML03P1 study demonstrated wide inter-patient variation (˜2 log fold) in CD33 intensity on leukemic cells with higher expression correlating with negative prognostic factors such as FLT3-ITD (15). A follow-up study in a larger AAML0531 cohort randomly assigned to GO or standard chemotherapy arm (No-GO) reported that addition of GO is more likely to benefit patients with higher leukemic CD33 intensity, as compared to patients with low CD33 expression where no benefit of adding GO was observed (16).

Coding and regulatory polymorphisms in CD33 have previously been sequenced and identified by us (17,28). In the AAML031 study, rs12459419, rs2455069 and rs1803254 were associated with leukemic cell surface CD33 expression and rs35112940 was predictive of EFS and RR in patients who received GO (17). Since that AAML031 clinical trial did not have a randomized control arm, it was not possible to compare the impact of CD33 SNPs in patients who did or did not receive GO-based chemotherapy. AAML0531, being a randomized study, allowed investigation of the influence of CD33 SNPs on treatment outcome by comparing patients who did or did not receive GO.

CD33 SNPs, particularly rs12459419, were a significant determinant of whether patients would or would not benefit from addition of GO to conventional chemotherapy. Patients with CC genotype for rs12459419, which is also associated with higher cell surface expression of CD33 WT isoform had almost 50% reduction in the risk of relapse and experience superior DFS with addition of GO to standard chemotherapy. Conversely for patients with at least one T allele, which results in alternate splicing with deletion of exon 2 and hence loss of IgV, no difference in outcome was observed with or without addition of GO. Since multiple SNPs were associated with cell surface expression and outcome, a CD33 SNP score was created. Patients with a CD33SNP score of ≥0 (around 50% of patients) have survival benefit with better EFS, DFS and lower risk of relapse when treated with GO containing chemotherapy as compared to standard arm. In patients with score <0, there was no benefit from addition of GO. This relationship of CD33SNP score was consistent in low and standard risk group patients and demonstrated a similar trend in high-risk group patients. However, this score was primarily driven by rs12459419 (or linked rs3865444) SNP. In fact for rs12459419 also, risk group analysis showed that low and standard risk group patients had significantly better outcome in CC genotype and similar trend in high-risk group patients. In fact in Low risk group patients overall survival (OS) from study entry was 90±7% when patients had CC genotype and were given GO based therapy, in contrast CC patients in No-GO arm had OS 73±11% (Table 9).

TABLE 9
CD33 SNPrs12459419 and clinical outcome in all patients and within each risk
group by treatment arm NO-GO vs. GO in patients treated under COG-AAML0531 study.
rs12459419
Low Risk

| | CC (N = 159) | | | CT (N = 119) | | | TT (N = 28) | | |
|---|---|---|---|---|---|---|---|---|---|
| | No-GO | GO | | No-GO | GO | | No-GO | GO | |
| | (N = 76) | (N = 83) | No-GO | (N = 61) | (N = 58) | No-GO | (N = 16) | (N = 12) | No-GO |
| | % ± | % ± | vs. GO | % ± | % ± | vs. GO | % ± | % ± | vs. GO |
| | 2SE % | 2SE % | p | 2SE % | 2SE % | p | 2SE % | 2SE % | p |
| 5 yr OS from | 73 ± 11% | 90 ± 7% | 0.014 | 88 ± 8% | 74 ± 12% | 0.058 | 88 ± 17% | 83 ± 22% | 0.761 |
| study entry | | | | | | | | | |
| 5 yr EFS from | 55 ± 12% | 82 ± 9% | 0.001 | 73 ± 11% | 64 ± 13% | 0.276 | 60 ± 26% | 58 ± 28% | 0.929 |
| study entry | | | | | | | | | |
| | No-GO | GO | No-GO | No-GO | GO | No-GO | No-GO | GO | No-GO |
| | (N = 65) | (N = 69) | vs. GO | (N = 46) | (N = 48) | vs. GO | (N = 14) | (N = 11) | vs. GO |
| 5 yr DFS from | 57 ± 13% | 84 ± 9% | 0.001 | 72 ± 13% | 70 ± 14% | 0.794 | 61 ± 28% | 55 ± 30% | 0.683 |
| End of Course 1 | | | | | | | | | |
| 5 yr RR from | 37 ± 13% | 10 ± 8% | <0.001 | 26 ± 13% | 19 ± 11% | 0.483 | 39 ± 30% | 45 ± 32% | 0.683 |
| End of Course 1 | | | | | | | | | |
| 5 yr TRM from | 6 ± 6% | 6 ± 6% | 0.944 | 2 ± 4% | 11 ± 10% | 0.107 | 0 ± 0% | 0 ± 0% | 1 |
| End of Course 1 | | | | | | | | | |

Standard Risk

| | CC (N = 192) | | | CT (N = 149) | | | TT (N = 44) | | |
|---|---|---|---|---|---|---|---|---|---|
| | No-GO | GO | | No-GO | GO | | No-GO | GO | |
| | (N = 101) | (N = 91) | No-GO | (N = 72) | (N = 77) | No-GO | (N = 22) | (N = 22) | No-GO |
| | % ± | % ± | vs. GO | % ± | % ± | vs. GO | % ± | % ± | vs. GO |
| | 2SE % | 2SE % | p | 2SE % | 2SE % | p | 2SE % | 2SE % | p |
| 5 yr OS from | 49 ± 11% | 56 ± 11% | 0.599 | 64 ± 12% | 60 ± 12% | 0.936 | 59 ± 21% | 64 ± 21% | 0.684 |
| study entry | | | | | | | | | |
| 5 yr EFS from | 33 ± 10% | 42 ± 10% | 0.397 | 44 ± 12% | 44 ± 12% | 0.532 | 31 ± 20% | 47 ± 23% | 0.197 |
| study entry | | | | | | | | | |
| | No-GO | GO | No-GO | No-GO | GO | No-GO | No-GO | GO | No-GO |
| | (N = 68) | (N = 67) | vs. GO | (N = 40) | (N = 60) | vs. GO | (N = 14) | (N = 15) | vs. GO |
| 5 yr DFS from | 38 ± 12% | 51 ± 12% | 0.181 | 55 ± 16% | 46 ± 14% | 0.721 | 43 ± 26% | 50 ± 28% | 0.439 |
| End of Course 1 | | | | | | | | | |
| 5 yr RR from | 59 ± 12% | 41 ± 12% | 0.056 | 43 ± 16% | 52 ± 14% | 0.635 | 57 ± 28% | 50 ± 30% | 0.441 |
| End of Course 1 | | | | | | | | | |
| 5 yr TRM from | 3 ± 4% | 8 ± 7% | 0.248 | 3 ± 5% | 2 ± 3% | 0.766 | 0 ± 0% | 0 ± 0% | 1 |
| End of Course 1 | | | | | | | | | |

High Risk

| | CC (N = 55) | | | CT (N = 45) | | | TT (N = 11) | | |
|---|---|---|---|---|---|---|---|---|---|
| | No-GO | GO | | No-GO | GO | | No-GO | GO | |
| | (N = 24) | (N = 31) | No-GO | (N = 24) | (N = 21) | No-GO | (N = 6) | (N = 5) | No-GO |
| | % ± | % ± | vs. GO | % ± | % ± | vs. GO | % ± | % ± | vs. GO |
| | 2SE % | 2SE % | p | 2SE % | 2SE % | p | 2SE % | 2SE % | p |
| 5 yr OS from | 46 ± 23% | 40 ± 19% | 0.767 | 32 ± 20% | 62 ± 21% | 0.056 | 56 ± 50% | — | 0.639 |
| study entry | | | | | | | | | |
| 5 yr EFS from | 29 ± 19% | 23 ± 15% | 0.222 | 21 ± 18% | 48 ± 22% | 0.037 | — | — | 0.981 |
| study entry | | | | | | | | | |
| | No-GO | GO | No-GO | No-GO | GO | No-GO | No-GO | GO | No-GO |
| | (N = 10) | (N = 14) | vs. GO | (N = 14) | (N = 16) | vs. GO | (N = 4) | (N = 4) | vs. GO |
| 5 yr DFS from | 30 ± 29% | 43 ± 26% | 0.5 | 28 ± 26% | 56 ± 25% | 0.112 | — | — | 0.62 |
| End of Course 1 | | | | | | | | | |
| 5 yr RR from | 70 ± 32% | 36 ± 27% | 0.073 | 65 ± 31% | 44 ± 26% | 0.261 | 25 ± 50% | 25 ± 50% | 0.919 |
| End of Course 1 | | | | | | | | | |
| 5 yr TRM from | 0 ± 0% | 21 ± 23% | 0.129 | 7 ± 14% | 0 ± 0% | 0.286 | 0 ± 0% | 25 ± 53% | 0.33 |
| End of Course 1 | | | | | | | | | |

Although functional consequences of CD33 SNPs are still under investigation, the promoter SNP rs3865444 A>G was identified in a genome-wide association analysis to be associated with late-onset of Alzheimer's (29) and later confirmed by other studies (30-33). The variant G allele was also shown to be associated with low CD33 expression levels in the microglial cells (34,35), which is consistent with our results in AML patients. Rs3865444 occurs in LD with rs12459419 (Ala14Val), which has recently been associated with transcript lacking exon 2 (22-24). CD33 alternately spliced transcripts characterized by skipping of exon 2 lacks IgV domain (21). As shown in FIG. 6A, the shorter CD33 variant lacks the IgV domain and thus would not be detected by hP67.7 antibody, as the epitope for hP67.7 lies in the IgV domain. Results on RNA transcript levels in diagnostic samples from AML patients presented herein confirm that the presence of T allele is associated with higher levels of alternatively spliced D2 isoform. GO targets the IgV domain, in consensus with the observation of lack of improvement in outcome with addition of GO in patients with variant T allele. By contrast, the HIM-34 antibody targets the IgC2 domain and hence would detect both CD33-WT and shorter CD33-D2 isoform (exon 2 skipped) (21).

Although in depth mechanistic study of rs12459419 and other SNPs in CD33 is warranted, our results show that CD33 rs12459419 genotype allows for identification of patients expressing CD33 isoform lacking the antibody binding site for GO and holds promise as a marker to select patients likely to benefit from addition GO to chemotherapy (CC genotype) regardless of clinical risk or surface CD33 expression level. This opens up opportunities to utilize patient genotypes for selection of CD33 targeted therapies. Additionally the fact that presence of splicing SNP results in shorter CD33 isoform lacking IgV domain warrants further investigation in developing CD33 immunoconjugates with epitope targeted to regions not influenced by splicing and/or CD33 genotypes, thus being effective in larger patient cohort. The results of our study would also be relevant to other CD33 targeted therapies such as SGN-33A, lintuzumab etc. Since genotype calls are stable as compared to use of CD33 mRNA or cell surface levels which is sensitive to-type of antibody utilized, storage conditions of specimens, heterogeneity of cell populations if not sorted or enriched; use of genotype as a marker for identification of patients will enhance clinical translation of CD33 genetics and will be adaptable in clinical practice.

### REFERENCES

1. Walter R B, Appelbaum F R, Estey E H, Bernstein I D. Acute myeloid leukemia stem cells and CD33-targeted immunotherapy. Blood 2012; 119(26):6198-208.
2. Bross P F, Beitz J, Chen G, Chen X H, Duffy E, Kieffer L, et al. Approval summary: gemtuzumab ozogamicin in relapsed acute myeloid leukemia. Clinical cancer research: an official journal of the American Association for Cancer Research 2001; 7(6):1490-6.
3. Petersdorf S H, Kopecky K J, Slovak M, Willman C, Nevill T, Brandwein J, et al. A phase 3 study of gemtuzumab ozogamicin during induction and postconsolidation therapy in younger patients with acute myeloid leukemia. Blood 2013; 121(24):4854-60.
4. Burnett A K, Hills R K, Hunter A E, Milligan D, Kell W J, Wheatley K, et al. The addition of gemtuzumab ozogamicin to low-dose Ara-C improves remission rate but does not significantly prolong survival in older patients with acute myeloid leukaemia: results from the LRF AML14 and NCRI AML16 pick-a-winner comparison. Leukemia: official journal of the Leukemia Society of America, Leukemia Research Fund, UK 2013; 27(1):75-81.
5. Burnett A K, Hills R K, Milligan D, Kjeldsen L, Kell J, Russell N H, et al. Identification of patients with acute myeloblastic leukemia who benefit from the addition of gemtuzumab ozogamicin: results of the MRC AML15 trial. Journal of clinical oncology: official journal of the American Society of Clinical Oncology 2011; 29(4):369-77.
6. Burnett A K, Russell N H, Hills R K, Kell J, Freeman S, Kjeldsen L, et al. Addition of gemtuzumab ozogamicin to induction chemotherapy improves survival in older patients with acute myeloid leukemia. Journal of clinical oncology: official journal of the American Society of Clinical Oncology 2012; 30(32):3924-31.
7. Castaigne S, Pautas C, Terre C, Raffoux E, Bordessoule D, Bastie J N, et al. Effect of gemtuzumab ozogamicin on survival of adult patients with de-novo acute myeloid leukaemia (ALFA-0701): a randomised, open-label, phase 3 study. Lancet 2012.
8. Thol F, Schlenk R F. Gemtuzumab ozogamicin in acute myeloid leukemia revisited. Expert opinion on biological therapy 2014; 14(8):1185-95.
9. Hills R K, Castaigne S, Appelbaum F R, Delaunay J, Petersdorf S, Othus M, et al. Addition of gemtuzumab ozogamicin to induction chemotherapy in adult patients with acute myeloid leukaemia: a meta-analysis of individual patient data from randomised controlled trials. The lancet oncology 2014; 15(9):986-96.
10. Loke J, Khan J N, Wilson J S, Craddock C, Wheatley K. Mylotarg has potent anti-leukaemic effect: a systematic review and meta-analysis of anti-CD33 antibody treatment in acute myeloid leukaemia. Annals of hematology 2015; 94(3):361-73.
11. Cooper™, Franklin J, Gerbing R B, Alonzo T A, Hurwitz C, Raimondi S C, et al. AAML03P1, a pilot study of the safety of gemtuzumab ozogamicin in combination with chemotherapy for newly diagnosed childhood acute myeloid leukemia: a report from the Children's Oncology Group. Cancer 2012; 118(3):761-9.
12. Gamis A S, Alonzo T A, Meshinchi S, Sung L, Gerbing R B, Raimondi S C, et al. Gemtuzumab ozogamicin in children and adolescents with de novo acute myeloid leukemia improves event-free survival by reducing relapse risk: results from the randomized phase III Children's Oncology Group trial AAML0531. Journal of clinical oncology: official journal of the American Society of Clinical Oncology 2014; 32(27):3021-32.
13. Clarke W T, Marks P W. Gemtuzumab ozogamicin: is there room for salvage? Blood 2010; 116(14):2618-9.
14. Ravandi F, Estey E H, Appelbaum F R, Lo-Coco F, Schiffer C A, Larson R A, et al. Gemtuzumab ozogamicin: time to resurrect? Journal of clinical oncology: official journal of the American Society of Clinical Oncology 2012; 30(32):3921-3.
15. Pollard J A, Alonzo T A, Loken M, Gerbing R B, Ho P A, Bernstein I D, et al. Correlation of CD33 expression level with disease characteristics and response to gemtuzumab ozogamicin containing chemotherapy in childhood AML. Blood 2012.
16. Pollard J A, Loken M, Gerbing R B, Raimondi S C, Hirsch B A, Aplenc R, et al. CD33 Expression and Its Association With Gemtuzumab Ozogamicin Response: Results From the Randomized Phase III Children's Oncology Group Trial AAML0531. Journal of clinical oncology: official journal of the American Society of Clinical Oncology 2016; 34(7):747-55.
17. Mortland L, Alonzo T A, Walter R B, Gerbing R B, Mitra A K, Pollard J A, et al. Clinical significance of CD33 nonsynonymous single-nucleotide polymorphisms in pediatric patients with acute myeloid leukemia treated with gemtuzumab-ozogamicin-containing chemotherapy. Clinical cancer research: an official journal of the American Association for Cancer Research 2013; 19(6):1620-7.
18. Pollard J A, Alonzo T A, Loken M, Gerbing R B, Ho P A, Bernstein I D, et al. Correlation of CD33 expression level with disease characteristics and response to gemtuzumab ozogamicin containing chemotherapy in childhood AML. Blood 2012; 119(16):3705-11.
19. Wells D A L, M.R. Flow cytometric mean fluorescence intensity: The biophysics behind the number. Leukemia research 2008; 32(6).
20. Hernandez-Caselles T, Martinez-Esparza M, Perez-Oliva A B, Quintanilla-Cecconi A M, Garcia-Alonso A, Alvarez-Lopez D M, et al. A study of CD33 (SIGLEC-3) antigen expression and function on activated human T and N K cells: two isoforms of CD33 are generated by alternative splicing. J Leukoc Biol 2006; 79(1):46-58.
21. Perez-Oliva A B, Martinez-Esparza M, Vicente-Fernandez J J, Corral-San Miguel R, Garcia-Penarrubia P, Hernandez-Caselles T. Epitope mapping, expression and post-translational modifications of two isoforms of CD33 (CD33M and CD33m) on lymphoid and myeloid human cells. Glycobiology 2011; 21(6):757-70.
22. Raj T, Ryan K J, Replogle J M, Chibnik L B, Rosenkrantz L, Tang A, et al. CD33: increased inclusion of exon 2 implicates the Ig V-set domain in Alzheimer's disease susceptibility. Hum Mol Genet 2014; 23(10):2729-36.
23. Malik M, Chiles J, 3rd, Xi H S, Medway C, Simpson J, Potluri S, et al. Genetics of CD33 in Alzheimer's disease and acute myeloid leukemia. Hum Mol Genet 2015; 24(12):3557-70.
24. Malik M, Simpson J F, Parikh I, Wilfred B R, Fardo D W, Nelson P T, et al. CD33 Alzheimer's risk-altering polymorphism, CD33 expression, and exon 2 splicing. J Neurosci 2013; 33(33):13320-5.
25. Jager E, van der Velden V H, te Marvelde J G, Walter R B, Agur Z, Vainstein V. Targeted drug delivery by gemtuzumab ozogamicin: mechanism-based mathematical model for treatment strategy improvement and therapy individualization. PloS one 2011; 6(9):e24265.
26. Jedema I, Barge R M, van der Velden V H, Nijmeijer B A, van Dongen J J, Willemze R, et al. Internalization and cell cycle-dependent killing of leukemic cells by Gemtuzumab Ozogamicin: rationale for efficacy in CD33-negative malignancies with endocytic capacity. Leukemia: official journal of the Leukemia Society of America, Leukemia Research Fund, UK 2004; 18(2):316-25.
27. Walter R B, Gooley T A, van der Velden V H, Loken M R, van Dongen J J, Flowers D A, et al. CD33 expression and P-glycoprotein-mediated drug efflux inversely correlate and predict clinical outcome in patients with acute myeloid leukemia treated with gemtuzumab ozogamicin monotherapy. Blood 2007; 109(10):4168-70.
28. Lamba J K, Pounds S, Cao X, Downing J R, Campana D, Ribeiro R C, et al. Coding polymorphisms in CD33 and response to gemtuzumab ozogamicin in pediatric patients with AML: a pilot study. Leukemia: official journal of the Leukemia Society of America, Leukemia Research Fund, UK 2009; 23(2):402-4.
29. Naj A C, Jun G, Beecham G W, Wang L S, Vardarajan B N, Buros J, et al. Common variants at MS4A4/MS4A6E, CD2A P, CD33 and EPHA1 are associated with late-onset Alzheimer's disease. Nat Genet 2011; 43(5):436-41.
30. Bao J, Wang X J, Mao Z F. Associations Between Genetic Variants in 19p13 and 19q13 Regions and Susceptibility to Alzheimer Disease: A Meta-Analysis. Med Sci Monit 2016; 22:234-43.
31. Omoumi A, Fok A, Greenwood T, Sadovnick A D, Feldman H H, Hsiung G Y. Evaluation of late-onset Alzheimer disease genetic susceptibility risks in a Canadian population. Neurobiol Aging 2014; 35(4):936 e5-12.
32. Tan L, Yu J T, Zhang W, Wu Z C, Zhang Q, Liu Q Y, et al. Association of GWAS-linked loci with late-onset Alzheimer's disease in a northern Han Chinese population. Alzheimers Dement 2013; 9(5):546-53.
33. Carrasquillo M M, Belbin O, Hunter T A, Ma L, Bisceglio G D, Zou F, et al. Replication of EPHA1 and CD33 associations with late-onset Alzheimer's disease: a multi-centre case-control study. Mol Neurodegener 2011; 6(1):54.
34. Bradshaw E M, Chibnik L B, Keenan B T, Ottoboni L, Raj T, Tang A, et al. CD33 Alzheimer's disease locus: altered monocyte function and amyloid biology. Nat Neurosci 2013; 16(7):848-50.
35. Griciuc A, Serrano-Pozo A, Parrado A R, Lesinski A N, Asselin C N, Mullin K, et al. Alzheimer's disease risk gene CD33 inhibits microglial uptake of amyloid beta. Neuron 2013; 78(4):631-43.

1. A method of treating a subject with a cancer expressing CD33 comprising:
performing an assay to detect the genotype of the subject for the CD33 single-nucleotide polymorphism rs12459419, wherein the genotype may be CC, TC, or TT, or detect the genotype of the subject for CD33 single-nucleotide polymorphism rs3865444, wherein the genotype may be CC, CA, or AA; and
administering a therapeutically effective amount of an agent that selectively binds to CD33 if the subject exhibits a CC genotype for the CD33 single-nucleotide polymorphism rs12459419 or rs3865444.

2. The method of claim 1, wherein the agent that selectively binds to CD33 comprises an antibody that selectively binds CD33, or an antigen binding fragment thereof.

3. (canceled)

4. The method of claim 1, wherein the agent that selectively binds to CD33 comprises an antibody that selectively binds CD33, or an antigen binding fragment thereof, conjugated to a toxin.

5-10. (canceled)

11. The method of claim 1, wherein the cancer is any one of acute lymphoblastic leukemia (ALL), acute promyelocytic leukemia (APL), and acute myeloid leukemia (AML).

12-13. (canceled)

14. The method of claim 1, wherein the subject has one or more of: the presence of blast cells that express CD33 within the hematopoietic system; leukostasis; anemia; leukopenia; neutropenia; thrombocytopenia; chloroma; granulocytic sarcoma; and myeloid sarcoma.

15. A method for determining whether a subject with a cancer expressing CD33 is likely to benefit from treatment with an agent that selectively binds to CD33 comprising:
providing tissue from a subject who has been diagnosed with the cancer;
performing an assay on the tissue, or on a derivative of the tissue, to detect the genotype of the subject for the CD33 single-nucleotide polymorphism rs12459419, wherein the genotype may be CC, TC, or TT or for the CD33 single-nucleotide polymorphism rs3865444, wherein the genotype may be CC, CA, or AA;
wherein the subject is likely to benefit from treatment with an agent that selectively binds to CD33 if the subject exhibits a CC genotype for the CD33 single-nucleotide polymorphism rs12459419 or the CD33 single-nucleotide polymorphism rs3865444 and the subject is not likely to benefit from treatment with an agent that selectively binds to CD33 if the subject exhibits a TC or TT genotype for the CD33 single-nucleotide polymorphism rs12459419 or a CA or AA genotype for the CD33 single-nucleotide polymorphism rs3865444.

16. The method of claim 15, wherein the agent that selectively binds to CD33 comprises an antibody that selectively binds CD33, or an antigen binding fragment thereof.

17. The method of claim 15, wherein the agent that selectively binds to CD33 selectively binds to amino acids encoded by exon 2 of CD33.

18. The method of claim 15, wherein the agent that selectively binds to CD33 is gemtuzumab ozogamicin, hP67.7, or SGN-33A.

19-24. (canceled)

25. The method of claim 15, wherein the subject has one or more of: the presence of blast cells that express CD33 within the hematopoietic system; leukostasis; anemia; leukopenia; neutropenia; thrombocytopenia; chloroma; granulocytic sarcoma; and myeloid sarcoma.

26. A method for detecting a polymorphism, comprising:
obtaining a biological sample of a subject that has a cancer expressing CD33, and
(i) performing an assay to detect the genotype of the subject for the CD33 single-nucleotide polymorphism (SNP) rs12459419 controlling expression of exon 2, wherein the genotype may be CC, TC, or TT and wherein the presence of the CC genotype in rs12459419 indicates expression of exon 2 of CD33; or
(ii) performing an assay to detect the presence of amino acids encoded by exon 2 of CD33, wherein the presence of amino acids encoded by exon 2 of CD33 indicates expression of exon 2 of CD33 and presence of a CC genotype in single-nucleotide polymorphism rs12459419 of CD33; or
(iii) performing an assay to detect the genotype of the subject for the CD33 single-nucleotide polymorphism (SNP) rs3865444 controlling expression of exon 2, wherein the genotype may be CC, CA, or AA and wherein the presence of the CC genotype in rs3865444 indicates expression of exon 2 of CD33.

27-35. (canceled)

36. The method of claim 26, wherein the subject has one or more of: the presence of blast cells that express CD33 within the hematopoietic system; leukostasis; anemia; leukopenia; neutropenia; thrombocytopenia; chloroma; granulocytic sarcoma; and myeloid sarcoma.

37-38. (canceled)

39. The method of claim 26, wherein the cancer is acute myeloid leukemia (AML), acute lymphoblastic leukemia (ALL), or acute promyelocytic leukemia (APL).

40-42. (canceled)

43. A kit comprising:
an agent that selectively binds to CD33, and
instructions indicating the use of the agent to treat a subject if the genotype of the subject for the CD33 single-nucleotide polymorphism rs12459419 or the CD33 single-nucleotide polymorphism rs3865444 is CC.

44. The kit of claim 43, wherein the agent that selectively binds to CD33 comprises an antibody that selectively binds CD33, or an antigen binding fragment thereof.

45. (canceled)

46. The kit of claim 43, wherein the agent that selectively binds to CD33 comprises an antibody that selectively binds CD33, or an antigen binding fragment thereof, conjugated to a toxin.

47-50. (canceled)

51. A method for determining a CD33SNP_Score for a subject comprising:
determining genotype scores of the subject for the CD33 single-nucleotide polymorphisms (SNPs) rs12459419 or rs3865444, rs1803254, rs35112940, and rs2455069, wherein
the genotype score for a single nucleotide polymorphism (SNP) with two wild-type alleles is 0,
the genotype score separately for each of the SNPs rs12459419, rs3865444, rs1803254, and rs35112940 with one wild-type allele and one variant allele is −1,
the genotype score for the SNP rs2455069 with one wild-type allele and one variant allele is 1,
the genotype score separately for each of the SNPs rs12459419, rs3865444, rs1803254, and rs35112940 with two variant alleles is −2, and
the genotype score for the SNP rs2455069 with two variant alleles is 2, and
adding the genotype scores to yield the CD33SNP_Score.

52-62. (canceled)

63. A method for determining whether a subject with cancer is likely to benefit from treatment with an agent that selectively binds to CD33 comprising:
determining a CD33SNP_Score for the subject, wherein
tissue is provided from the subject who has been diagnosed with the cancer;
an assay is performed on the tissue, or on a derivative of the tissue, to detect the genotype of the subject for the CD33 single-nucleotide polymorphisms rs12459419 or rs3865444, rs1803254, rs35112940, and rs2455069, wherein the wild-type, single variant, or double variant genotype, respectively may be:
CC, TC, or TT for rs12459419,
CC, CA, or AA for rs3865444,
GG, CG, or CC for rs1803254,
GG, AG, or AA for rs35112940, or
AA, AG, or GG for rs2455069, and
wherein the subject is likely to benefit from treatment with an agent that selectively binds amino acids encoded by exon 2 of CD33 if the CD33SNP_Score for the subject is greater than or equal to zero.

64-69. (canceled)

70. A method for determining whether a subject with cancer expressing CD33 is likely to benefit from treatment with an agent that selectively binds to CD33 comprising:
providing tissue from a subject who has been diagnosed with the cancer;
performing an assay on the tissue, or on a derivative of the tissue, to detect the CD33 single-nucleotide polymorphism genotype of the subject for the CD33 single-nucleotide polymorphism rs12459419 or the CD33 single-nucleotide polymorphism rs3865444, and
determining the single-nucleotide polymorphism genotype score, wherein the genotype score may be 0, −1, or −2, wherein a score of 0 indicates that the subject is likely to benefit from treatment.

71-77. (canceled)

78. A method of treating a subject with a cancer expressing CD33 comprising:
performing an assay to detect the genotype of the subject for any one of the CD33 single-nucleotide polymorphisms rs1354106, rs3852865, and rs12985029, wherein the genotype may be wild-type, heterozygous variant, or homozygous variant; and
administering a therapeutically effective amount of an agent that selectively binds to CD33 if the subject exhibits a wild-type genotype for the CD33 single-nucleotide polymorphism rs1354106, rs3852865, or rs12985029.

79-122. (canceled)
